SEQUENCE LISTING (23 sequences)

SEQ ID NO: 1 - DNA, 20 bp, Artificial Sequence
  Description of Artificial Sequence Synthetic primer
tggggytttacrggtaacct

SEQ ID NO: 2 - DNA, 21 bp, Artificial Sequence
  Description of Artificial Sequence Synthetic primer
aacrcgcttaacaaagcactc

SEQ ID NO: 3 - DNA, 24 bp, Artificial Sequence
  Description of Artificial Sequence Synthetic probe
tagttgtgatgcwatcatgactag

SEQ ID NO: 4 - DNA, 22 bp, Artificial Sequence
  Description of Artificial Sequence Synthetic primer
taatcagacaaggaactgatta

SEQ ID NO: 5 - DNA, 19 bp, Artificial Sequence
  Description of Artificial Sequence Synthetic primer
cgaaggtgtgacttccatg

SEQ ID NO: 6 - DNA, 20 bp, Artificial Sequence
  Description of Artificial Sequence Synthetic probe
gcaaattgtgcaatttgcgg

SEQ ID NO: 7 - DNA, 21 bp, Artificial Sequence
  Description of Artificial Sequence Synthetic primer
ccctgtgggttttacacttaa

SEQ ID NO: 8 - DNA, 19 bp, Artificial Sequence
  Description of Artificial Sequence Synthetic primer
acgattgtgcatcagctga

SEQ ID NO: 9 - DNA, 28 bp, Artificial Sequence
  Description of Artificial Sequence Synthetic probe
ccgtctgcggtatgtggaaaggttatgg

SEQ ID NO: 10 - DNA, 22 bp, Artificial Sequence
  Description of Artificial Sequence Synthetic primer
ggggaacttctcctgctagaat

SEQ ID NO: 11 - DNA, 22 bp, Artificial Sequence
  Description of Artificial Sequence Synthetic primer
cagacattttgctctcaagctg

SEQ ID NO: 12 - DNA, 20 bp, Artificial Sequence
  Description of Artificial Sequence Synthetic probe
ttgctgctgcttgacagatt

SEQ ID NO: 13 - RNA, 26 nt, Severe acute respiratory syndrome-related coronavirus
ucucuaaacgaacuuuaaaaucugug

SEQ ID NO: 14 - RNA, 26 nt, Severe acute respiratory syndrome coronavirus 2
ucucuaaacgaacuuuaaaaucugug

SEQ ID NO: 15 - RNA, 13 nt, Severe acute respiratory syndrome-related coronavirus
caacuaaacgaac

SEQ ID NO: 16 - RNA, 13 nt, Severe acute respiratory syndrome coronavirus 2
caacuaaacgaac

SEQ ID NO: 17 - RNA, 15 nt, Severe acute respiratory syndrome-related coronavirus
cacauaaacgaacuu

SEQ ID NO: 18 - RNA, 15 nt, Severe acute respiratory syndrome coronavirus 2
cacauaaacgaacuu

SEQ ID NO: 19 - RNA, 13 nt, Severe acute respiratory syndrome-related coronavirus
ugaguacgaacuu

SEQ ID NO: 20 - RNA, 13 nt, Severe acute respiratory syndrome coronavirus 2
ugaguacgaacuu

SEQ ID NO: 21 - RNA, 18 nt, Severe acute respiratory syndrome-related coronavirus
ggucuaaacgaacuaacu

SEQ ID NO: 22 - RNA, 18 nt, Severe acute respiratory syndrome coronavirus 2
ggucuaaacgaacuaaau

SEQ ID NO: 23 - protein, 6 aa, Artificial Sequence
  Description of Artificial Sequence Synthetic 6xHis tag
HHHHHH

(19) United States
(12) Patent Application Publication
(10) Pub. No.: US 2022/0380769 A1
(43) Pub. Date: Dec. 1, 2022

(54) METHODS, COMPOSITIONS, AND VACCINES FOR TREATING A VIRUS INFECTION

(71) Applicant: AIM ImmunoTech Inc., Ocala, FL (US)

(72) Inventors: Thomas K. EQUELS, Ocala, FL (US); David R. STRAYER, Bryn Mawr, PA (US); Diane L. YOUNG, Yardville, NJ (US)

(73) Assignee: AIM ImmunoTech Inc., Ocala, FL (US)

(21) Appl. No.: 17/773,538

(22) PCT Filed: Jan. 25, 2021
(86) PCT No.: PCT/US2021/014969

§ 371 Date: Apr. 29, 2022

Related U.S. Application Data
(60) Provisional application No. 62/965,713, filed on Jan. 24, 2020.
(60) Provisional application No. 62/967,493, filed on Jan. 29, 2020.
(60) Provisional application No. 62/969,572, filed on Feb. 3, 2020.
(60) Provisional application No. 62/971,199, filed on Feb. 6, 2020.
(60) Provisional application No. 62/976,994, filed on Feb. 14, 2020.
(60) Provisional application No. 62/982,641, filed on Feb. 27, 2020.
(60) Provisional application No. 62/993,514, filed on Mar. 23, 2020.
(60) Provisional application No. 62/994,777, filed on Mar. 25, 2020.
(60) Provisional application No. 63/003,197, filed on Mar. 31, 2020.
(60) Provisional application No. 63/016,960, filed on Apr. 28, 2020.
(60) Provisional application No. 63/026,712, filed on May 18, 2020.
(60) Provisional application No. 63/029,395, filed on May 22, 2020.
(60) Provisional application No. 63/092,432, filed on Oct. 15, 2020.
(60) Provisional application No. 63/125,950, filed on Dec. 15, 2020.

(51) Int. Cl. C12N 15/117; A61K 31/713; A61K 9/00; A61K 9/06; A61K 9/08; A61K 9/107; A61K 9/127; A61K 9/16; A61K 9/19

(52) U.S. Cl. CPC C12N 15/117; A61K 31/713; A61K 9/0014; A61K 9/0021; A61K 9/0043; A61K 9/006; A61K 9/0075; A61K 9/0078; A61K 9/008; A61K 9/0095; A61K 9/06; A61K 9/08; A61K 9/107; A61K 9/127; A61K 9/1605; A61K 9/19; C12N 2310/17

(57) ABSTRACT

This disclosure relates to a method of treating, preventing, or reducing a symptom of a virus infection, including a SARS-CoV-2 infection, by administering an effective amount of tdsRNA optionally with an anti-viral agent, to a subject.

### CROSS-REFERENCE TO RELATED APPLICATIONS

This application claims the benefit of priority to U.S. Provisional Application No. 62/965,713 filed Jan. 24, 2020, U.S. Provisional Application No. 62/967,493 filed Jan. 29, 2020, U.S. Provisional Application No. 62/969,572 filed Feb. 3, 2020, U.S. Provisional Application No. 62/971,199 filed Feb. 6, 2020, U.S. Provisional Application No. 62/976,994 filed Feb. 14, 2020, U.S. Provisional Application No. 62/982,641 filed Feb. 27, 2020, U.S. Provisional Application No. 62/993,514 filed Mar. 23, 2020, U.S. Provisional Application No. 62/994,777 filed Mar. 25, 2020, U.S. Provisional Application No. 63/003,197 filed Mar. 31, 2020, U.S. Provisional Application No. 63/016,960 filed Apr. 28, 2020, U.S. Provisional Application No. 63/026,712 filed May 18, 2020, U.S. Provisional Application No. 63/029,395 filed May 22, 2020, U.S. Provisional Application No. 63/092,432 filed Oct. 15, 2020, and U.S. Provisional Application No. 63/125,950 filed Dec. 15, 2020, the entire content of which is incorporated herein by reference.

### BACKGROUND

This disclosure generally relates to Therapeutic dsRNAs (referred to herein as “tdsRNA” whether it is singular or plural) which can be administered to a subject for beneficial effects. These beneficial effects include the prevention or treatment of a virus infection such as a SARS-CoV-2 infection.

At the end of 2019, a novel coronavirus was identified as the cause of a cluster of pneumonia cases in Wuhan, China. It rapidly spread and became a global pandemic. The disease was named novel coronavirus disease 2019 (COVID-19) and the causative agent was discovered to be a coronavirus later named SARS-CoV-2. SARS-CoV-2 is a member of the β coronavirus family. It is the seventh known coronavirus to infect humans; four of these coronaviruses (229E, NL63, OC43, and HKU1) cause slight symptoms of the common cold. The other three (3 coronaviruses, SARS-CoV, MERS-CoV, and SARS-CoV-2 can cause severe symptoms and even death, with fatality rates of 10%, 37%, and 5%, respectively. Over 400,000 people in the U.S. and over 2 million people worldwide have died from SARS-CoV-2 infection.

While COVID-19 has been the subject of numerous studies, there is no evidence that any potential therapy can improve outcomes in patients. Therefore, the current treatments for SARS-CoV-2 infections are mainly directed to supportive care. For these reasons, there is a long-felt need for methods and compositions for treating SARS-CoV-2 infections.

### BRIEF DESCRIPTION

One embodiment is directed to composition for treating or preventing a viral infection caused by a virus in a subject, wherein the composition comprises a therapeutic double-stranded RNA (tdsRNA), and wherein the tdsRNA may be at least one selected from the group consisting of rIₙ.r(CₓU)ₙ (formula 1); rIₙ.r(CₓG)ₙ (formula 2); rAₙ.rUₙ (formula 3); rIₙ.rCₙ (formula 4); and rugged dsRNA (formula 5); wherein x may be at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

The virus in any embodiment of the disclosure, including all the compositions (products) and methods and processes disclosed anywhere, may be any virus. Preferably, the virus is a virus that infects a mammalian host such as a human host. For example, the virus may be a virus as listed in Table 2 of this disclosure. As another example, the virus may be any strain or variant of any virus as listed in Table 2 such as a coronavirus. The coronavirus may be SARS-CoV-2 or a variant or a substrain or a mutant thereof. As another example, the virus may be at least one selected from the group consisting of Human coronavirus 229E (HCoV-229E); Human coronavirus NL63 (HCoV-NL63, New Haven coronavirus); Human coronavirus OC43 (HCoV-OC43); Human coronavirus HKU1; Middle East respiratory syndrome-related coronavirus (MERS-CoV); novel coronavirus 2012 (HCoV-EMC); Severe acute respiratory syndrome-related coronavirus (SARS-CoV); Severe acute respiratory syndrome coronavirus 2 (SARS-CoV-2); Ebola Virus; H5 influenza; H7 influenza; H5N1 influenza; Influenza A; Influenza B; H1N1 influenza; H3N2 influenza; H7N9 influenza; H5N6 influenza; H10N8 influenza; H9N2 influenza; H6N1 influenza; West Niles Virus; and Zika Virus.

In the formula for tdsRNA for any embodiment of the disclosure, “n” may be a number selected from the group consisting of: 40 to 50,000; 40 to 40,000; 50 to 10,000; 60 to 9000; 70 to 8000; 80 to 7000; and 380 to 450.

In any embodiment comprising the composition, at least 90 wt % of the tdsRNA may be larger than a size selected from the group consisting of: 40 basepairs; 50 basepairs; 60 basepairs; 70 basepairs; 80 basepairs; and 380 basepairs.

In any embodiment comprising the composition, at least 90 wt % of the tdsRNA may be smaller than a size selected from the group consisting of: 50,000 basepairs; 40,000 basepairs, 10,000 basepairs; 9000 basepairs; 8000 basepairs; 7000 basepairs; and 450 basepairs.

The tdsRNA in any embodiment of this disclosure may be represented by formula 1 to 5 and wherein n is from 40 to 40,000; wherein the tdsRNA has about 4 to about 4000 helical turns of duplexed RNA strands; or wherein the tdsRNA has a molecular weight selected from the group consisting of: 2 kDa to 30,000 kDa; 25 kDa to 2500 kDa; and 250 kDa to 320 kDa.

In any embodiment, the tdsRNA may comprise rIₙ.ribo(C₁₁₋₁₄U)ₙ; and rugged dsRNA.

In any embodiment, the rugged dsRNA may have a single strand comprised of r(C₄₋₂₉U)ₙ, r(C₁₁₋₁₄U)ₙ, or r(C₁₂U)ₙ; and an opposite strand comprised of r(I); wherein the single strand and the opposite strand do not base pair the position of the uracil base, and wherein the single strand and the opposite strand are partially hybridized.

In any embodiment, the rugged dsRNA may have (1) a molecular weight of about 250 kDa to 500 kDa; (2) two strands of RNA wherein each strand of the rugged dsRNA is from about 400 to 800 basepairs in length; or (3) about 30 to 100, or 30 to 60 helical turns of duplexed RNA.

In any embodiment, the rugged dsRNA may be resistant to denaturation under conditions that are able to separate hybridized poly(riboinosinic acid) and poly(ribocytosinic acid) strands (rIₙ.rCn).

In any embodiment, the rugged dsRNA may be an isolated double-stranded ribonucleic acid (dsRNA) enzymatically active under thermal stress comprising the following characteristics: (1) each strand may have a molecular weight of about 250 KDa to about 500 KDa, a length of 400-800 basepairs, or about 30 to 60 helical turns of duplex RNA, (2) a single strand comprised of poly(ribocytosinic₄₋₂₉ uracilic acid) and an opposite strand comprised of poly(riboinosinic acid), (3) wherein the two strands do not base pair the position of the uracil base, (4) wherein the two strands base pair the position of the cytosine base, and (5) wherein said strands are partially hybridized.

In any embodiment, the tdsRNA may be produced by a method that comprises: (a) synthesizing a first single-stranded RNA (first ssRNA) in a first synthesis reaction with PNPase as the only RNA polymerase, and purifying said first ssRNA after the first synthesis reaction; (b) synthesizing a second single-stranded RNA (second ssRNA) in a second synthesis reaction with PNPase as the only RNA polymerase, and purifying said second ssRNA after the second synthesis reaction; and (c) hybridizing the first ssRNA with the second ssRNA at 62° C. to 68° C. for 5 to 30 minutes and then 50° C. for more than 30 minutes to form the tdsRNA. In the method, step a) and step b) may be performed in any order; the first synthesis reaction comprises inosine diphosphate (rIDP) as the only free ribonucleotide; and wherein the second synthesis reaction comprises cytidine diphosphate (rCDP) and uridine diphosphate (rUDP) as the only two free ribonucleotides. The a molar ratio of (free rCDP):(free rUDP) in the second synthesis reaction is about (11 to 14):(1).

In a preferred embodiment, the tdsRNA in the composition may comprise 0.1-12 mol %, 0.1-10 mol %, 0.1-8 mol %, 0.1-5 mol %, 0.1-3 mol %, or 0.1-2 mol % rugged dsRNA. In any embodiment, the composition may comprise at least one pharmaceutically acceptable carrier.

In any embodiment, the tdsRNA may be complexed with a stabilizing polymer. The stabilizing polymer may be at least one selected from the group consisting of: polylysine; polylysine and carboxymethylcellulose; polyarginine; polyarginine and carboxymethylcellulose; and any combination thereof.

In any embodiment, the composition comprising tdsRNA may further comprise an antiviral agent which is not a tdsRNA. The antiviral agent may be an antibody to an S protein of SARS-CoV-2; an antibody to a NTD region of a S protein of SARS-CoV-2; an antibody to a HR1 region of a S protein of SARS-CoV-2; an antibody to a RBD region of a S protein of SARS-CoV-2; a SARS-CoV monoclonal antibody; a MERS-CoV monoclonal antibody; a SARS-CoV-2 monoclonal antibody; a peptide; a protease inhibitor; a PIKfyve inhibitor; a TMPRSS2 inhibitor; a cathepsin inhibitor; a furin inhibitor; an antiviral peptide; an antiviral protein; an antiviral chemical compound; and an antiviral agent. For example, the antiviral agent may be at least one selected from the group consisting of: 1A9; 201; 311mab-31B5; 311mab-32D4; 47D11; 4A8; 4C2; 80R; Apilimod; B38; camostat mesylate; Casirivimab; CR3014; CR3022; D12; E-64D; EK1; EK1C4; H4; HR2P; IBP02; Imdevimab; m336; MERS-27; MERS-4; MI-701; n3088; n3130; P2B-2F6; P2C-1F11; PI8; S230; S309; SARS-CoV-2 S HR2P fragment (aa1168-1203); Tetrandrine; Viracept (nelfinavir mesylate); YM201636; α-1-PDX; favipiravir; IFN-α; IFN-α1b; IFN-α2a; lopinavir-ritonavir; Q-Griffithsin (Q-GRFT); and Griffithsin; oseltamivir; zanamivir; abacavir; zidovudine; zalcitabine; didanosine; stavudine; efavirenz; indinavir; ritonavir; nelfinavir; amprenavir; ribavirin; Remdesivir; chloroquine; hydroxychloroquine; rIFN-alpha-2a; rIFN-beta-1b; rIFN-gamma; nIFN-alpha; nIFN-beta; nIFN-gamma; IL-2; PD-L1; Anti-PD-L1; a checkpoint inhibitor; an interferon; interferon mixture; recombinant or natural interferon; Alferon; alpha-interferon species; recombinant or natural interferon alpha; recombinant or natural interferon alpha 2a; recombinant or natural interferon beta; recombinant or natural interferon beta 1b; and recombinant or natural interferon gamma. The alpha-interferon species may be a mixture of at least seven species of alpha-interferon produced by human white blood cells, wherein the seven species are: interferon alpha 2; interferon alpha 4; interferon alpha 7; interferon alpha 8; interferon alpha 10; interferon alpha 16; and interferon alpha 17.

In any embodiment, the composition may be at least one selected from the group consisting of an aqueous solution, a powder, a dry particle, a liquid particle, a gel particle, a semidry particle, an isotonic formulation, and a composition for nasal administration.

Another embodiment is directed to a vaccine composition, wherein the composition is a composition comprising (1) tdsRNA as described in this disclosure and (2) wherein composition further comprises a vaccine against a virus. The virus may be any virus described in this disclosure. The vaccine may comprise at least one selected from the group consisting of: an inactivated virus, an attenuated virus, a virus antigen, and a messenger RNA encoding protein comprising a virus antigen. For example, the virus antigen may be an antigen from the S, E, M, or N structural protein of a coronavirus such as a SARS-CoV-2 coronavirus.

One embodiment is directed to a method for treating a viral infection caused by a virus in a subject comprising: determining that the subject is infected by the virus; and administering an effective amount of a composition comprising tdsRNA as described anywhere in this disclosure to the subject infected by the virus. In this method, the subject may be a subject that has been infected by the virus for not more than two to seven days, or up to 14 days.

Another embodiment is directed to a method for preventing a viral infection caused by a virus in a subject comprising: determining that the subject is not infected by the virus; and administering an effective amount of a composition comprising tdsRNA as described anywhere in this disclosure to the subject not infected by the virus.

Another embodiment is directed to a method for treating a viral infection caused by a virus in a subject. The method comprises the step of administering an effective amount of a composition comprising tdsRNA as described anywhere in this disclosure to the subject who has (1) been infected with the virus, (2) at risk for being infected by the virus because of exposure a second subject infected with the virus, or (3) is at risk for being infected by the virus because of presence in an area where there are reported cases of virus infection.

Another embodiment is directed to a method for immunizing a subject against a viral infection caused by a virus. The method comprises the step of administering to the subject at least a first compound and a second compound in any order together or separately wherein the first compound comprises an effective amount of a vaccine, and the second compound is an effective amount of a composition comprising tdsRNA as described anywhere in this disclosure. In a preferred embodiment, the method produces an immune response in the subject. For example, the immune response may be at least one selected from the group consisting of virus-specific immunoglobulin production; virus specific IgG production; virus specific IgG1 production; virus specific IgG2a production; virus specific IgA production; and virus specific IgM production. In this embodiment, the method may induce an increased cross-reactive immune response and cross protection against a second different virus in a subject. The second different virus may be a variant, a different strain, or a mutation of the virus (i.e., the first virus). In a preferred embodiment, the method provides a vaccine effect that is superior than a viral antigen (e.g., coronavirus antigen) administered alone. That is, the immune response is more than the sum of (1) tdsRNA administered alone, (2) viral antigen alone. In other words, there is a synergistic effect between the tdsRNA and the viral antigen. The synergistic effect may be measure by measuring, for example, the amount of virus specific antibody titer (e.g., IgA, IgG etc.). As discussed, the first compound and second compound may be administered together as a mixture; or wherein the first compound and second compound may be administered at the same time; or separately at different times. When the compounds are administered at a different time, the first compound and the second compound may be administered within a time period selected from the group consisting of: 2 months; 1 month; 3 weeks; 2 weeks; 1 week; 3 days; 1 day; 12 hours, 6 hours, 3 hours, 2 hours, 1 hour, and 30 minutes.

In any embodiment, the vaccine may comprise at least one selected from the group consisting of: an inactivated virus, an attenuated virus, a virus antigen, and a messenger RNA encoding a virus antigen. The virus antigen may be an antigen from the S, E, M, or N structural protein of a coronavirus such as a SARS-CoV-2 coronavirus.

In any embodiment, when the “virus” or the “second different virus” is discussed, the “virus” or the “second different virus” may be a coronavirus and preferably a SARS-CoV-2 virus. Further, the “virus” or the “second different virus” may be a virus of Table 2, or a virus selected from the group consisting of Human coronavirus 229E (HCoV-229E); Human coronavirus NL63 (HCoV-NL63, New Haven coronavirus); Human coronavirus OC43 (HCoV-OC43); Human coronavirus HKU1; Middle East respiratory syndrome-related coronavirus (MERS-CoV); novel coronavirus 2012 (HCoV-EMC); Severe acute respiratory syndrome-related coronavirus (SARS-CoV); Severe acute respiratory syndrome coronavirus 2 (SARS-CoV-2); Ebola Virus; H5 influenza; H7 influenza; H5N1 influenza; Influenza A; Influenza B; H1N1 influenza; H3N2 influenza; H7N9 influenza; H5N6 influenza; H10N8 influenza; H9N2 influenza; H6N1 influenza; West Niles Virus; and Zika Virus. The virus may be a mutant variant, clade, subclade of any of the discussed virus.

In any embodiment, the “effective amount” may be a therapeutically effective amount or a prophylactically effective amount of the tdsRNA.

In any embodiment, administering may be at least one administering method selected from the group consisting of: intravenous administration; intradermal administration; subcutaneous administration; intramuscular administration; intranasal administration (pulmonary airway administration); intranasal administration and oral administration (i.e., both together); intraperitoneal administration; intracranial administration; intravesical administration; oral administration (through the mouth, by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration; and a combination thereof. Intranasal administration may be administering to nasal passages; administering to nasal epithelium; administering to lung; administering by inhalation; administering to the larynx; administering to bronchi; administering to alveoli; administering by inhalation; administering by nasal instillation; and a combination thereof.

In any embodiment, administering may be administering to at least one tissue or cell selected from the group consisting of: an airway tissue; nose tissue; oral tissue; alveoli tissue; pharynx tissue; trachea tissue; bronchi tissue; carina tissue; bronchi tissue; bronchioles tissue; lung tissue; lobe of a lung tissue; alveoli tissue; nasal passage tissue; nasal epithelium tissue; larynx tissue; bronchi tissue; inhalation tissue; an epithelium cell; an airway epithelium cell; a ciliated cell; a goblet cell; a non-ciliated cell; a basal cell; a lung cell; a nasal cell; a tracheal cell; a bronchial cell; a bronchiolar epithelial cell; an alveolar epithelial cell; and a sinus cell.

In any embodiment, administering may be administering by at least one delivery system selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal aerosol device; a nasal nebulization device; a pressure-driven jet nebulizer; ultrasonic nebulizer; a breath-powered nasal delivery device; a atomized nasal medication device; an inhaler; a powder dispenser; a dry powder generator; an aerosolizer; an intrapulmonary aerosolizer; a sub-miniature aerosolizer; a propellant based metered dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and a combination thereof.

In a preferred embodiment, the methods of the disclosure may reduce nasal virus titer at least 10 fold or 100 fold, or prevents or reduces nasal shedding of virus at least 10 fold or 100 fold. We note that nasal virus titer may exist even for a person which has been vaccinated or who is immune to a virus because a temporary cache of virus may exist in the oral or nasal cavity just due to exposure to a virus source such as aerosol droplets from an infected person.

In any embodiment, administering may be at a dosage of about 25-700 milligram, 20 mg to 200 mg, 50 mg to 150 mg, or 80 mg to 140 mg, per day.

In any embodiment, the subject may be a mammal, preferably a host of the virus, and most preferably a human.

Another embodiment is directed to a delivery system or medical device encompassing a composition, including vaccine composition, of the disclosure. Another embodiment is directed to a delivery system or medical device as discussed wherein the delivery system or medical device is selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal nebulization device; a pressure-driven jet nebulizer; ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered dose inhaler; a dry powder inhalation device; an instillation device; an intranasal instillation device; an intravesical instillation device; a swab; a pipette; a nasal irrigation device; a nasal rinse; an aerosol device; a metered aerosol device; a pressurized dosage device; a powdered aerosol device; a spray aerosol device; a spray device; a metered spray device; a suspension spray device; and a combination thereof.

Various embodiments are described throughout this disclosure and it is envisioned that any embodiment may be combined with any other embodiment to create an additional embodiment of the disclosure. The phrase “any embodiment” includes, at least, the compositions, tdsRNAs including all the formulas, the methods, the vaccines and the devices of the disclosure.

### DETAILED DESCRIPTION

### 1: Overview

SARS-CoV-2

The present disclosure provides double-stranded RNA (referred to herein as “therapeutic double-stranded RNA” or “tdsRNA” and described in more detail below) and methods for using these dsRNAs (tdsRNA) in the treatment (e.g., therapeutic and/or prophylactic, vaccination) of SARS-CoV-2 infection. Methods for treating or reducing at least one symptom of SARS-CoV-2 infections and preventing spread of the virus and further infection are objectives of the present disclosure.

There is an urgent need for treatment of subjects who have been exposed to SARS-CoV-2 but have not developed symptoms of COVID-19 (SARS-CoV-2 infection) (e.g., fever, dry cough and difficulty breathing). The period between exposure and symptoms may be one to five days, one week, 10 days, two weeks or more. Timely treatment may allow a subject to reduce the time of viral infection and, if treatment was applied early enough, escape symptoms altogether. For example, if a patient was determined to have been exposed to a virus such as SARS-CoV-2, the patient can be treated with the methods and compositions (tdsRNA) of this disclosure to reduce the chances of a SARS-CoV-2 infection (SARS-CoV-2 infection) or to reduce or eliminate one or all of the symptoms associated with the infection.

There is also an urgent need for preventing infection from SARS-CoV-2 for example, by administering an agent with a prophylactic effect on a subject. That is, after treatment, the subject would be less susceptible to infection with SARS-CoV-2.

SARS-CoV-2 is a single-stranded RNA-enveloped virus. The first isolate has an RNA genome of 29,881 bases in length (GenBank no. MN908947) and encodes about 9860 amino acids. The S, E, M, and N genes encode structural proteins, whereas nonstructural proteins, such as 3-chymotrypsin-like protease, papain-like protease, and RNA-dependent RNA polymerase, are encoded by the ORF region.

A large number of glycosylated S proteins cover the surface of SARS-CoV-2 and bind to the host cell receptor angiotensin-converting enzyme 2 (ACE2), mediating viral entry into cells. When the S protein binds to the receptor, Transmembrane (TM) protease serine 2 (TMPRSS2), a type 2 TM serine protease located on the host cell membrane, promotes virus entry into the cell by activating the S protein. Once the virus enters the cell, the viral RNA is released, polyproteins are translated from the RNA genome, and replication and transcription of the viral RNA genome occur via protein cleavage and assembly of the replicase-transcriptase complex. Viral RNA is replicated, and structural proteins are synthesized, assembled, and packaged in the host cell, after which viral particles are released.

These S proteins are important to the viral life cycle and provide potential targets for drug therapies. Due to its central functions in SARS-CoV-2 infection, it represents one of the most popular targets for COVID-19 vaccine and therapeutic research.

The important role of the S protein in viral infection indicates that it is a potential target for vaccine development, antibody-blocking therapy, and small molecule inhibitors. See, e.g., discussions of the S protein in the Example section of this disclosure.

The various products and methods of this disclosure would be especially useful and effective for subjects at a heightened risk of contracting the disease. They may be those who are in close proximity (e.g., presence within about two meters or in the same enclosed room) to an infected individual. For example, prophylactic treatment would be useful for healthcare workers, public safety workers, police, airport workers, teachers, students, transportation workers and people traveling into or out of an infected region (i.e., between an infected region and an uninfected region). For example, the first diagnosed case of SARS-CoV-2 infection in the US had exposed over 40 individuals to the virus before he was hospitalized. Currently, there are thousands of persons in quarantine globally because of possible exposure to the virus.

### 2: Definitions

### Definitions

“COVID-19” is a disease caused by an infection of the “SARS-CoV-2” virus. These names were standardized by the World Health Organization (WHO) and the International Committee on Taxonomy of Viruses (ICTV). Because of novelty and the rapidity of SARS-CoV-2 spread worldwide, a number of names have been used for SARS-CoV-2 including: COVID-19, COVID-19 virus, 2019-nCoV, Novel coronavirus pneumonia, Wuhan coronavirus, and severe acute respiratory syndrome coronavirus 2 (SARS-CoV-2). A subject with the disease of COVID-19 would have an infection of SARS-CoV-2 virus and, conversely, a subject with an infection of SARS-CoV-2 has the disease COVID-19. However, a subject with a SARS-CoV-2 infection may be asymptomatic or may have not yet developed all the symptoms.

This disclosure relates to, inter alia, tdsRNA. tdsRNA can also be called “therapeutic dsRNA,” or “therapeutic double-stranded RNA” and these terms have the same meaning.

“r” and “ribo” have the same meaning and refer to ribonucleic acid or the nucleotide or nucleoside that are the building block of ribonucleic acid.

RNA consists of a chain of linked units called nucleotides. This disclosure relates mostly to RNA and, therefore, unless otherwise specified, the nucleotides and bases expressed refers to the ribo form of the nucleotide or base (i.e., ribonucleotide with one or more phosphate groups). Therefore “A” refers to rA or adenine, “U” refers to rU or uracil, “C” refers to rC or cytosine, “G” refers to rG or guanine, “I” refers to rI or inosine, “rN” refers to rA, rU, rC, rG or rI. Each of these (i.e., A, U, C, G, I) may have one or more phosphate groups as discussed above depending on whether they are part of a chain (i.e., RNA) or free (nucleoside, nucleotide, etc.).

“n” is a positive number and refers to the length of a ssRNA or dsRNA or to the average length of a population of ssRNA or dsRNA. “n” can be a positive integer when referring to one nucleic acid molecule or it can be any positive number when it is an average length of a population of nucleic acid molecules.

A free nucleotide is a nucleotide that has not been incorporated into an RNA chain. The free nucleotide may be incorporated into an RNA chain by an enzyme such as an RNA polymerase, and, after such incorporation, the nucleotide is no longer considered a free nucleotide. Examples of free nucleotides would include free rA, free rU, free rC, free rG, free rI, free rN.

And RNA may have a ratio of nucleotides or bases. For example, r(C₁₂U)ₙ denotes a single RNA strand that has, on average 12 C bases or nucleotides for every U base or nucleotide. As another example, r(C₁₁₋₁₄U)ₙ denotes a single RNA strand that has, on average 12 C bases or nucleotides for every U base or nucleotide.

Formulas: As an example, the formula “rIₙ.r(C₁₂U)ₙ” can be expressed as riboIₙ.ribo(C₁₂U)ₙ, rIₙ.ribo(C₁₂U)ₙ, or riboIₙ.r(C₁₂U)ₙ, refers to a double-stranded RNA with two strands. One strand (rIₙ) is poly ribo-inosine of n bases in length. The other strand is ssRNA of random sequence of C and U bases, the random sequence ssRNA is n bases in length, and a ratio of C bases to U bases in the random sequence ssRNA is about 12 (i.e., mean 12 C to 1 U).

The “.” symbol indicates that one strand of the dsRNA is hybridized (hydrogen-bonded) to the second strand of the same dsRNA. Therefore, rIₙ.r(C₁₂U)ₙ is double-stranded RNA comprising two ssRNA. One ssRNA is poly(I) and the other ssRNA is poly(C₁₂U). It should be noted that while we referred to the two strands being hybridized, not 100% of the bases form base pairing as there are some bases that are mismatches. Also, because rU does not form base pairing with rI as well as rC form base paring with rI, rU provides a focus of hydrodynamic instability in rIₙ.r(C₁₂U)ₙ at the locations of the U bases.

As another example, the formula “rIₙ.r(C₁₁₋₁₄U)ₙ” refers to the same dsRNA except that a ratio of C bases to U bases one strand is about 11 to about 14. That is, the ratio can be 11, 12, 13 or 14 or any value between 11 and 14. For example, when half of the strands are r(C₁₂U)ₙ and half of the strands are r(C₁₃U)ₙ, the formula would be r(C₁₂₅U)ₙ.

In any part of this disclosure and in any embodiment, the terms “comprise,” “comprising,” “consist,” “consisting,” “consist essentially of,” or “consisting essentially of” can be replaced with each other and each of these replacements is also an embodiment of the disclosure. That is, the interchange of one of the above listed term of this paragraph with another, even though they have different meanings, is also envisioned.

As discussed earlier, the term “r” and “ribo” has the same meaning in the formulas of the disclosure. Thus, rI, riboI, r(I) and ribo(I) refer to the same chemical which is the ribose form of inosine. Similarly, rC, riboC, r(C) and ribo(C) all refer to cytidine in the ribose form which is a building block of RNA. rU, riboU, r(U) and ribo(U) all refer to Uracil in the ribose form which is a building block of RNA.

In this disclosure, PNPase refers to Polynucleotide Phosphorylase (PNPase) and not to another, otherwise unrelated enzyme, Purine nucleoside phosphorylase.

A nucleotide triphosphate (rNTP), as used herein, refers to a molecule including a nucleobase linked to a ribose (i.e., nucleoside) and three phosphates (i.e. nucleotide). A nucleotide diphosphate (rNDP) refers to the same molecule, but which has two phosphate moieties. A nucleotide monophosphate (rNMP) refers to the same molecule, but which has one phosphate moiety. The nucleotide monophosphate, diphosphate, and triphosphate are sometimes referred to herein as rNMP, rNDP, and rNTP, respectively. The N in rNMP, rNDP and rNTP refers to any nucleotide, including naturally occurring nucleotides, synthetic nucleotides, and modified nucleotides. Thus the terms rNMP, rNDP and rNTP refer to nucleotide monophosphate, nucleotide diphosphate and nucleotide triphosphate, respectively. These terms (rNMP, rNDP and rNTP) also refer to any nucleotide having any naturally occurring, synthetic, or modified nucleotide therein. The methods and products of this disclosure are all RNAs and thus unless otherwise noted all references to nucleic acids are referring to the ribose form. That is, unless otherwise noted, NMP, NDP, NTP, IMP, IDP, ITP, CMP, CDP, CTP, UMP, UDP, UTP refers to rNMP, rNDP, rNTP, rIMP, rIDP, rITP, rCMP, rCDP, rCTP, rUMP, rUDP, rUTP respectively.

In this disclosure, inosine is also considered a possible rNMP, rNDP, or rNTP. Inosine is a nucleoside that is formed when hypoxanthine is attached to a ribose ring (also known as a ribofuranose) via a β-N9-glycosidic bond.

Nucleotide monophosphates include at least adenosine monophosphate (AMP or rAMP), guanosine monophosphate (GMP or rGMP), cytidine monophosphate (CMP or rCMP), uridine monophosphate (UMP or rUMP), and inosine monophosphate (IMP rIMP). Nucleotide diphosphates include at least adenosine diphosphate (ADP or rADP), guanosine diphosphate (GDP or rGDP), cytidine diphosphate (CDP or rCDP), uridine diphosphate (UDP or rUDP), and inosine diphosphate (IDP or rIDP).

Nucleotide triphosphates include at least adenosine triphosphate (ATP or rATP), guanosine triphosphate (GTP or rGTP), cytidine triphosphate (CTP or rCTP), uridine triphosphate (UTP or rUTP), and inosine triphosphate (ITP or rITP).

In a preferred embodiment, the tdsRNA produce by the methods on this disclosure may comprise no detectable ssRNA (more than 0% to less than 0.1%). In a preferred embodiment, the tdsRNA may comprises between 0.1% to 4% ssRNA, between 0.5% to 3% ssRNA, and preferably between 1.5% to 2.5% ssRNA. The ssRNA may be present at the ends of the tdsRNA—in other words, the ends of the tdsRNA may be single-stranded (also called “sticky ends”). The single-stranded region may be a 5′ overhang or a 3′ overhang. A single-stranded region may also be internal such as if the bases are not paired due to temperature or salt conditions or if one strand is longer than the complementary strand causing a loop structure where one strand of the double-stranded RNA comprises an internal loop of single-stranded RNA.

While this disclosure refers to dsRNA and tdsRNA, it is not required that the tdsRNA comprising only two ssRNA in duplex. For example, tdsRNA may comprise one strand of 300 bases and (1) two opposite strands of 150 bases each, or three opposite strands of 100 bases each.

The dsRNA (tdsRNA) and ssRNA of this disclosure are homopolymers (e.g., a single-stranded RNA where every base is the same) or heteropolymers (e.g., a single-stranded RNA where the bases can be different) of limited base composition. The tdsRNA are not mRNA and are distinct from mRNA in structure. For example, the ssRNA and dsRNA are preferably missing one or all of the following: (1) 5′ cap addition, (2) polyadenylation, (3) start codon, (4) stop codon, heterogeneous protein-coding sequences, and (5) spice signals.

As used herein, the term “substantially free” is used operationally, in the context of analytical testing of the material. Preferably, purified material is substantially free of one or more impurities. In a preferred embodiment, the tdsRNA of this disclosure is substantially free (e.g., more than 0% to less than 0.1%) or completely free (0%) of dI/dI dsRNA or dCdU/dCdU dsRNA. In other words, the tdsRNA is substantially free or completely free (0%) of homodimers of polymer 1 or homodimers of polymer 2. Substantially free in this context would be considered to be more than 0% but less than 1%, less than 0.5%, less than 0.2%, less than 0.1%, or less than 0.01% of a contaminant such as (1) dI/dI (polymer 1/polymer 1) dsRNA, dCdU/dCdU (polymer 2/polymer 2) dsRNA.

The terms “intranasal” or “intranasally”, “instillation”, “instillation of a liquid”, “instillation using a sprayer” as used herein, refers to a route of delivery of an active compound to a patient by inhalation to the nasal mucosa, the airway, the lung or a combination thereof.

As used herein, the term “nebulizer” refers to a drug delivery device used to administer medication such as tdsRNA in the form of a mist inhaled into the central nervous system through the noses. Nebulizers may use oxygen, compressed air, ultrasonic power, mechanical means, etc. to break up medical solutions and suspensions into small aerosol droplets that can be directly inhaled from the device.

As used herein, the term “aerosol” refers to a mixture of gas and liquid particles, and the best example of a naturally occurring aerosol is mist, formed when small vaporized water particles mixed with hot ambient air are cooled down and condense into a fine cloud of visible airborne water droplets. In one embodiment of the present disclosure, an aerosol may be produced through an aerosol spray or a sprayer. As used herein, the term “aerosol spray” or “a sprayer” refers to a type of dispensing system which creates an aerosol mist of liquid particles. This is used with a can or bottle that contains a liquid under pressure. When the container's valve is opened, the liquid is forced out of a small hole and emerges as an aerosol or mist. As gas expands to drive out the payload, only some propellant evaporates inside the can to maintain an even pressure. Outside the can, the droplets of propellant evaporate rapidly, leaving the payload suspended as very fine particles or droplets. An atomizer is a similar device that is pressurized by a hand-operated pump rather than by stored gas.

A particle, droplet, or an aerosol, and the like in this description may be a liquid suspension particle or a dry particle. A dry particle may be dry as it is produced, as it is administered, and/or as it exits an apparatus. It can be a dry particle even if the particle, droplet, or an aerosol starts out as a liquid because the liquid may be a fast evaporating liquid. Therefore, by the time the particle, droplet, or an aerosol contacts the subject, it will be dry. Also, it is possible a liquid particle, droplet, or an aerosol will dry after contact with a subject, for example, by rapid evaporation of the liquid component.

The term “lyophilizate,” as used herein in connection with the formulation according to the disclosure, denotes a formulation which is manufactured by freeze-drying methods known in the art. The solvent (e.g., water) is removed by freezing following sublimation under vacuum and desorption of residual water at an elevated temperature. In the pharmaceutical field, the lyophilizate usually has a residual moisture of about 0.1 to 5% (w/w) and is present as a powder or a stable physical cake. The lyophilizate is characterized by dissolution after the addition of a reconstitution medium.

The terms “intranasal” or “intranasally,” “instillation,” “instillation of a liquid,” “instillation using a sprayer” as used herein, refers to a route of delivery of an active compound to a patient by inhalation to the nasal mucosa, the airway, the lung or a combination thereof. Inhalation may be by breathing through the mouth or through a stoma as a result of a tracheostomy.

Active ingredients or active agents are used interchangeably and include any active ingredient or active agent described in this disclosure including, at least, tdsRNA. Other active agents include, at least, interferons such as Alferon.

COVID-19 and SARS-CoV-2

The present disclosure provides double-stranded RNA (referred to herein as “therapeutic double-stranded RNA” or “tdsRNA” and described in more detail below) and methods for using these dsRNAs (tdsRNA) in the treatment (e.g., therapeutic and/or prophylactic) of a SARS-CoV-2 infection.

Thus, methods for treating SARS-CoV-2 infections, for vaccinating against SARS-CoV-2, and methods for preventing the spread of the virus and further infection are objectives of the present disclosure.

There is an urgent need for treatment of subjects who have been exposed to SARS-CoV-2 but have not developed symptoms of SARS-CoV-2 infection (e.g., fever, dry cough and difficulty breathing). The period between exposure and symptoms may be one to five days, one week, 10 days, two weeks or more. Timely treatment may allow a subject to reduce the time of viral infection and, if treatment was applied early enough, escape symptoms altogether. For example, if a patient was determined to have contact with SARS-CoV-2, the healthcare workers can be treated with the methods and compositions (tdsRNA) of this disclosure to reduce the chances of developing a SARS-CoV-2 infection (SARS-CoV-2 infection) (COVID-19 disease) or to reduce or eliminate the symptoms thereof.

Viral infection may be diagnosed in subjects to identify those who are affected and need treatment. While a large population can be rapidly screened for those having a high body temperature, such screening is neither specific nor sensitive. Better detection of infection for diagnosis is preferred: (i) PCR or serologic assay for the presence of nucleic acids or proteins, respectively, specific for SARS-CoV-2 in a specimen obtained from a subject suspected of being infected (e.g., respiratory material such as nasopharyngeal or oropharyngeal swab, sputum, endotracheal aspirate, and bronchoalveolar lavage, serum or whole blood, and urine), or (ii) by the subject's symptoms such as high fever, dry cough, and difficulty breathing), leading to pneumonia and severe acute respiratory syndrome. Detection of endemic human coronaviruses (e.g., HCoV-229E, HCoV-NL63, HCoV-HKU1 and HCoV-OC43) is not required, but they should be excluded from diagnosis by using assay reagents specific for SARS-CoV-2. After sequencing a sufficient number of SARS-CoV-2 isolates and developing specific assay reagents (e.g., primers, probes, antibodies) therefrom, confirmatory sequencing should not be necessary.

There is also an urgent need for preventing infection from SARS-CoV-2 for example, by administering an agent with a prophylactic effect on a subject. That is, after treatment, the subject would be less susceptible to infection with SARS-CoV-2.

The various treatments would be especially useful and effective for subjects at a heightened risk of contracting the disease. They may be those who are in close proximity (e.g., presence within about two meters or in the same enclosed room) to an infected individual. For example, prophylactic treatment would be useful for healthcare workers, public safety workers, police, airport workers, teachers, students, transportation workers and people traveling into or out of an infected region (i.e., between an infected region and an uninfected region). For example, the first diagnosed case of SARS-CoV-2 infection in the US has exposed over 40 individuals to the virus before he was hospitalized. Currently, there are thousands of persons in quarantine globally because of possible exposure to the virus.

### 3: tdsRNA

The double-stranded RNAs described in this disclosure are therapeutic double-stranded RNA, abbreviated as “tdsRNA.” tdsRNA includes, at least, Rintatolimod which is a tdsRNA of the formula rIₙ.r(C₁₂U)ₙ). tdsRNA may be stored or administered in a pharmaceutically acceptable solution such as Phosphate Buffered Saline (PBS).

The tdsRNA may be a tdsRNA produced by any of the methods of this disclosure—referred to herein as the “tdsRNA Product” or “tdsRNA”—the two terms have the same meaning. tdsRNA can be represented by one or more of the formulas below in any combination:

rIₙ.r(CₓU)ₙ (formula 1)

rIₙ.r(CₓG)ₙ (formula 2)

rAₙ.rUₙ (also called polyA.polyU) (formula 3)

rIₙ.rCₙ (formula 4)

rugged dsRNA (formula 5)

Each will be discussed further below.

The tdsRNA may be represented by one or more of the formulas as follows:

rIₙ.r(CₓU)ₙ (formula 1)

rIₙ.r(CₓG)ₙ (formula 2)

x may be at least one selected from the group consisting of: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29 (4 to 29), 4-30 (4 to 30), 4-35 (4 to 35), 11-14 (11 to 14), 30-35 (30 to 35). Of these, x=12, and x=11-14 (x may be any value between 11 to 14) are especially preferred.

In these formulas 1 to 5, and in other formulas, where there is no subscript next to a base, the default value is “1.” For example, in the formula rIₙ.r(C₁₂U)ₙ, there is no subscript following “U,” it is understood that rIₙ.r(C₁₂U)ₙ is the same as rIₙ.r(C₁₂U₁)ₙ and the formula is meant to convey that for the strand denoted as r(C₁₂U₁)ₙ, there are 12 rC base for every rU base. Thus, x is also a ratio of the bases of one strand of the tdsRNA. The length of the tdsRNA strand is denoted as a lowercase “n” (e.g., rIₙ.r(C₁₂U)ₙ). The subscript n is also the length of each individual single-stranded nucleic acid. Since tdsRNA is double-stranded, n is also the length of the double-stranded nucleic acid—i.e., the length of the tdsRNA. For example, rIₙ.r(C₁₂U)ₙ indicates, inter alia, a double-stranded RNA with each strand with a length of n.

In another aspect, the tdsRNA may have a formula as follows:

rAₙ.rUₙ (also called polyA.polyU) (formula 3)

rIₙ.rCₙ (formula 4)

In another aspect, the tdsRNA may be a rugged dsRNA (formula 5).

In one embodiment, tdsRNA is one or more at least one selected from the group consisting of formula 1, formula 2, formula 3, formula 4, and formula 5. In another embodiment, tdsRNA comprises formula 1 and formula 2 only. In one preferred embodiment, tdsRNA comprises formula 1 only. In another embodiment, tdsRNA comprises formula 1 and formula 5 (rugged dsRNA) only.

In another aspect, at least 70%, at least 80%, or at least 90% of the tdsRNA may have a molecular weight of between 400,000 Daltons to 2,500,000 Daltons. Where the term percent (“%”) is used, the percent may be weight percent or molar percent.

In another aspect, the tdsRNA comprises a first ssRNA and a second ssRNA and each of these first ssRNA or second ssRNA may contain one or more strand breaks.

In another aspect, the tdsRNA may comprise at least one selected from the group consisting of: a 3′ overhang end, a 5′ overhang end, a blunt end, an internal ssRNA sequence, one or more strand breaks in a first ssRNA, and one or more strand breaks in a second ssRNA.

In another aspect, the tdsRNA is a linear molecule—that is a molecule that is not branched or that does not contain any loop structure. In different aspects, at least 60%, at least 70%, at least 80%, at least 90%, at least 95% or 100% of the tdsRNA is a linear molecule.

In another aspect, the tdsRNA has the property that greater than about 90%, greater than 95%, greater than 98%, greater than 99%, or 100% of the bases of the RNA are in a double-stranded configuration.

In any aspect, the tdsRNA may be in a therapeutic composition comprising, for example, a tdsRNA, and a pharmaceutically acceptable excipient (carrier).

One embodiment of tdsRNA is directed to rintatolimod, which is a tdsRNA of the formula rIₙ.r(C₁₂U)ₙ and which is also denoted by the trademark AMPLIGEN®.

In a preferred embodiment, the tdsRNA are of the general formula rIₙ.r(C₁₁₋₁₄, U)ₙ and are described in U.S. Pat. Nos. 4,024,222 and 4,130,641 (which are incorporated by reference herein) or synthesized according to this disclosure.

In the case where the tdsRNA is rAₙ.rUₙ, the tdsRNA may be matched (i.e., not in mismatched form).

tdsRNA (e.g., Rintatolimod) has undergone extensive clinical and preclinical testing. It has been well-tolerated in clinical trials enrolling over 1,200 patients with over 100,000 doses administered and there have been no drug-related deaths. Two placebo-controlled, randomized studies show no increase in serious adverse events compared to placebo. Favorable safety profiles have been seen for intraperitoneal, intravenous, and intranasal routes of administration of tdsRNA.

3.1 Length of tdsRNA

The length of the tdsRNA, may be represented by bases for one strand of the tdsRNA or in basepairs for both strands for the tdsRNA. It is understood that in some embodiments that not all of the bases (e.g., U and I) are in basepaired configuration. For example, rU bases do not pair as well as rC bases to inosine.

The length of the tdsRNA may be measured by (1) bases or basepairs, (2) molecular weight which is the weight of the double-stranded tdsRNA (e.g., Daltons) or (3) turns of the double-stranded RNA. These measurements can be easily interconverted. For example, it is generally accepted that there are about 629 Daltons per base pair.

“n” represents length in units of basepair or basepairs (abbreviated as bp regardless of whether it is singular or plural) for double-stranded nucleic acid. “n” can also represent bases for single-stranded RNA. Because “bp” represents singular or plural, it is the same as “bps” which is another representation of basepairs.

The tdsRNA can have the following values for its length “n” (in bases for single strand or basepairs for double strands): 4-5000, 10-50, 10-500, 10-40,000, 40-40,000, 40-50,000, 40-500, 50-500, 100-500, 380-450, 400-430, 400-800 or a combination thereof. Expressed in molecular weight, the tdsRNA may have the following values: 30 kDa to 300 kDa, 250 kDa to 320 kDa, 270 kDa to 300 kDa or a combination thereof. Expressed in helical turns, the tdsRNA may have 4.7 to 46.7 helical turns of duplexed RNA, 30 to 38 helical turns of duplexed RNA, 32 to 36 helical turns of duplexed RNA or a combination thereof.

The length may be an average basepair, average molecular weight, or an average helical turns of duplexed RNA and can take on integer or fractional values.

3.2 tdsRNA Preparation

One aspect is directed to a method for the synthesis of a therapeutic double-stranded RNA (tdsRNA). The method comprises the steps of a) synthesizing a first single-stranded RNA (first ssRNA) in a first synthesis reaction with PNPase as the only RNA polymerase, b) synthesizing a second single-stranded RNA (second ssRNA) in a second synthesis reaction with PNPase as the only RNA polymerase, and c) hybridizing the first ssRNA with the second ssRNA to form the tdsRNA, wherein step a) and step b) are performed in any order. Similarly, in any method for the synthesis of both strands of a tdsRNA, the order of strand synthesis may be in any order. “In any order” as used herein means that one strand may be synthesized before a second strand or vice versa. Further, both strands may be synthesized at the same time in different synthesis reactions in different vessels.

In the method above, or in any aspect of the disclosure, the first synthesis reaction and the second synthesis reaction comprise one or more reagents at least one selected from the group consisting of: tris(hydroxymethyl)aminomethane buffer, MgCl₂, EDTA, Urea and PNPase.

In any aspect of the disclosure, the first synthesis reaction may comprise inosine diphosphate (rIDP) as the only free ribonucleotide. One nonlimiting example of a product that can be made with this aspect is rIₙ.r(N)ₙ wherein r(N)ₙ represents a ssRNA or analog thereof of any sequence.

In any aspect of the disclosure, the second synthesis reaction may comprise cytidine diphosphate (rCDP) and uridine diphosphate (rUDP) as the only free ribonucleotides. In one aspect, the molar ratio of free rCDP/free rUDP in the second synthesis reaction may be any positive number but is preferably about 4 to 29, about 11 to 14, or about 12. Nonlimiting examples of products that can be made with this aspect include rIₙ.r(CₓU)ₙ, rIₙ.r(C₄₋₂₉U)ₙ, rIₙ.r(C₁₁₋₁₄U)ₙ, rIₙ.r(C₁₂U)ₙ, where x may be any non-zero number.

In any aspect of the disclosure, the second synthesis reaction may comprise cytidine diphosphate (rCDP) and guanosine diphosphate (rGDP) as the only free ribonucleotides. In one aspect, a molar ratio of free rCDP/free rGDP in the second synthesis reaction is about 4 to 29, about 11 to 14, or about 12. Nonlimiting examples of products that can be made with this aspect include rIₙ.r(GₓU)ₙ, rIₙ.r(G₄₋₂₉U)ₙ, rIₙ.r(G₁₁₋₁₄U)ₙ, rIₙ.r(G₁₂U)ₙ, where x may be any positive non zero number.

In any aspect of the disclosure, the second synthesis reaction comprises cytidine diphosphate (rCDP) as the only free ribonucleotide. A nonlimiting example of products that can be made with this aspect includes rIₙ.rCn.

In any aspect of the disclosure, the first synthesis reaction may comprise free Adenosine 5′-Diphosphate as the only free ribonucleotide, and the second synthesis reaction may comprise free Uridine 5′-Diphosphate as the only free ribonucleotide. A nonlimiting example of products that can be made with this aspect includes rAn.rUn.

In one aspect, the first synthesis reaction and the second synthesis reaction are performed in the absence of a number of reagents including adenosine triphosphate (ATP), rNMP (ribonucleotide mono phosphates), rNTP (ribonucleotide triphosphates), DNA (deoxynucleic acids), dNTP (deoxynucleotide triphosphates), dNDP (deoxynucleotide diphosphates), dNMP (deoxynucleotide monophosphates).

In any aspect of this disclosure, the methods may include one or more optional steps as follows: (1) purifying said first ssRNA after the first synthesis reaction and before the hybridizing step, (2) purifying said second ssRNA after the second synthesis reaction and before the hybridizing step. Purifying refers to purifying the first ssRNA or the second ssRNA from proteins such as PNPase and/or purifying the first ssRNA from the other components of the reaction including, for example, ribonucleotide diphosphates. PNPase may be added for example, in the range of 500-700 Units per Liter of reaction.

In any aspect of this disclosure, the hybridizing step may be performed by bringing a mixture of equal molar amounts of two ssRNA and incubating the two ssRNA in an aqueous solution at 62° C. to 68° C. for 5 to 30 minutes and then 50° C. for more than 30 minutes. The sum of the concentrations of the first ssRNA and the second ssRNA may be, for example, 7.9 mM. The hybridization solution may be, for example, sodium phosphate buffer (150 mM NaCl, 1 mM MgCl₂, 8 mM Na₂HPO₄, 1.6 mM NaH₂PO₄). Unless otherwise specified, this hybridization step can be used to form a dsRNA, which can be a tdsRNA, from two ssRNAs for any aspect of this disclosure.

In any aspect of this disclosure, the tdsRNA, dsRNA, or ssRNA may be purified by filtering with a 0.2-micron filter. For example, in the methods of the disclosure, the method may comprise an optional step which is to purify the RNA by filtering through a 0.2-micron filter after the hybridization step.

3.3 Rugged dsRNA (a Form of tdsRNA)

Rugged dsRNA is a tdsRNA that is resistant to denaturation under conditions that are able to separate hybridized poly(riboinosinic acid) and poly(ribocytosinic acid) strands (that is, rIₙ.rCₙ strands). See, U.S. Pat. Nos. 8,722,874 and 9,315,538 (incorporated by reference) for a further description of Rugged dsRNA and exemplary methods of preparing such molecules.

In one aspect, a rugged dsRNA can be an isolated double-stranded ribonucleic acid (dsRNA) which is resistant to denaturation under conditions that are able to separate hybridized poly(riboinosinic acid) and poly(ribocytosinic acid) strands, wherein only a single strand of said isolated dsRNA comprises one or more uracil or guanine bases that are not base-paired to an opposite strand and wherein said single strand is comprised of poly(ribocytosinic₃₀₋₃₅ uracilic acid). Further, the single strand may be partially hybridized to an opposite strand comprised of poly(riboinosinic acid). In another aspect, rugged dsRNA may be an isolated double-stranded ribonucleic acid (dsRNA) which is resistant to denaturation under conditions that are able to separate hybridized poly(riboinosinic acid) and poly(ribocytosinic acid) strands.

In another aspect, Rugged dsRNA, has at least one of the following: r(Iₙ).r(C₄₋₂₉U)ₙ, r(In)r(C₁₂U)ₙ, r(Iₙ).r(C₁₁₋₁₄U)ₙ, r(In)r(C₃₀U)ₙ, or r(In)r(C₃₀₋₃₅U)ₙ. In another aspect, Rugged dsRNA may have a size of 4 bps to 5000 bps, 40 bps to 500 bps, 50 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof.

In another aspect, Rugged dsRNA is produced by isolating the 5 minute HPLC peak of a tdsRNA preparation.

3.4 Rugged dsRNA Preparation

In one embodiment, the starting material for making Rugged dsRNA may be dsRNA prepared in vitro using conditions of this disclosure. For example, the specifically configured dsRNA described in U.S. Pat. Nos. 4,024,222, 4,130,641, and 5,258,369 (which are incorporated by reference herein) are generally suitable as starting materials after selection for rugged dsRNA. tdsRNA (or preparations of tdsRNA) described in this disclosure is also useful as starting material.

After procuring starting material, Rugged dsRNA may be isolated by at least subjecting the partially hybridized strands of a population of dsRNA to conditions that denature most dsRNA (more than 10 wt % or mol %, more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 95 wt % or mol %, or more than 98 wt % or mol %) in the population, and then selection negatively or positively (or both) for dsRNA that remain partially hybridized. The denaturing conditions to unfold at least partially hybridized strands of dsRNA may comprise an appropriate choice of buffer salts, pH, solvent, temperature, or any combination thereof. Conditions may be empirically determined by observation of the unfolding or melting of the duplex strands of ribonucleic acid. The yield of rugged dsRNA may be improved by partial hydrolysis of longer strands of ribonucleic acid, then selection of (partially) hybridized stands of appropriate size and resistance to denaturation.

The purity of rugged dsRNA, which functions as tdsRNA, may thus be increased from less than about 0.1-10 mol % (e.g., rugged dsRNA is present in at least 0.1 mol % or 0.1 wt percent but less than about 10 mol % or 10 wt percent) relative to all RNA in the population after synthesis to a higher purity. A higher purity may be more than 20 wt % or mol %, more than 30 wt % or mol %, more than 40 wt % or mol %, more than 50 wt % or mol %, more than 60 wt % or mol %, more than 70 wt % or mol %, more than 80 wt % or mol %, more than 90 wt % or mol %, more than 98 wt % or mol %, or between 80 to 98 wt % or mol %. All wt % or mol % is relative to all RNA present in the same composition.

Another method of isolating Rugged dsRNA is to employ chromatography. Under analytical or preparative high-performance liquid chromatography, Rugged dsRNA can be isolated from a preparation (e.g., the starting material as described above) to produce poly(I):poly(C₁₂U)ₙ (e.g., poly(I):poly(C₁₁₋₁₄U)ₙ) as a substantially purified and pharmaceutically-active molecule with an HPLC peak of about 4.5 to 6.5 minutes, preferably between 4.5 and 6 minutes and most preferably 5 minutes.

Rugged dsRNA and the method of making rugged dsRNA are described in U.S. Pat. Nos. 8,722,874 and 9,315,538 (incorporated by reference).

Comments Regarding all Embodiments and Formulas

For any of the embodiments, X is a ratio between two types of bases (i.e., C and U in this case), and therefore, x can have a non-integer value such as 12.5, 4.5, 9.6, 29.1 and the like. Since n represents the length for both strands, both strands of ssRNA are the same length which gives rise to a dsRNA with no significant single-stranded regions in the middle or at the end of the double-stranded structure.

The tdsRNA of this disclosure, which can be made using the methods of this disclosure, has a number of benefits. The tdsRNA of formula 1, for example, in the form of rintatolimod, is the only known specific TLR3 agonist based on synthetic double-stranded RNA (dsRNA) with a well-developed intravenous, intraperitoneal and intranasal safety profile. Toll-like Receptor 3 (TLR-3) is a primary natural danger signal in the body for viral infection.

tdsRNA of formula 1 and 5 has demonstrated antiviral activity against a broad spectrum of viruses, including Herpes viruses, Alphaviruses, Coronaviruses (as shown in this disclosure) and Filoviruses. Rintatolimod has shown a survival benefit in Alphaviruses, Coronaviruses, Filoviruses and Paramyxoviruses in animal models. In a mouse model of Ebola Virus Disease, Rintatolimod (6 mg/kg) produced 100% survival vs. 0% survival in the control animals.

Of the tdsRNA of the disclosure, the tdsRNA of formula 1 and formula 5 is preferred. This is because mis-paired regions of base pair hydrogen bonding accelerate chain hydrolysis while preserving biological activity compared to, for example, formula 3 and formula 4. Also formula 1 and formula 5 had decreased toxicity and improved safety compared to the tdsRNA which has an absence of mis-pairing. In usage, there is no development of antibodies to formula 1 and formula 5 while, in contrast, there is a 40-60% antibody formation with poly ICLC and poly ICL. Also, formula 1 and formula 5 is a selective TLR3 agonist compared to formula 4 species which also activates helicases (i.e. MDA-5). An additional benefit of the tdsRNA of formula 1 and formula 5 is that they are selective TLR-3 agonist (i.e. it does not activate the TLR-3 independent pathways using RNA helicase sensors such as MDAS or retinoic acid-inducible protein I (RIG-1)). Formula 1 and formula 5 does not induce TNF-α while formula 4 is non-selective and activates both MDAS and RIG-I using TLR-3 independent proinflammatory pathways. Formula 4 also induces high levels of TNF-α while Formula 1 and formula 5 does not.

Stabilizing Polymers

In any of the described embodiments, the tdsRNA may be complexed with a stabilizing polymer such as: polylysine, polylysine plus carboxymethylcellulose (lysine carboxy methyl cellulose), polyarginine, polyarginine plus carboxymethylcellulose, or a combination thereof. Some of these stabilizing polymers are described, for example, in U.S. Pat. No. 7,439,349.

Modified Backbone

The tdsRNA may comprise one or more alterations in the backbone of the nucleic acid. For example, configured tdsRNA may be made by modifying the ribosyl backbone of poly(riboinosinic acid) r(Iₙ), for example, by including 2′-O-methylribosyl residues. Specifically configured dsRNA may also be modified at the molecule's ends to add a hinge(s) to prevent slippage of the base pairs, thereby conferring specific bioactivity in solvents or aqueous environments that exist in human biological fluids.

Additional Agents

The tdsRNA of this disclosure may be in a compound or in a combination with a number of additional agents which are described herein.

### 4. Anti SARS-CoV-2 Agents

4.1 Proteins Agents and Chemical Agents

Optional components of the disclosed composition are proteins (including antibodies) and chemical agents that are therapeutic against SARS-CoV-2 infections. These components include, at least, the ones listed in the table below. It should be understood that a protein includes an antibody or a peptide. Also, reference to “protein” or “proteins” or chemicals also include derivatives thereof. A derivative of a protein may contain only the functional elements of the protein (i.e., functional fragments thereof). For example, a derivative of an antibody may be the binding fragment or binding parts only (e.g., F_{ab}). Other derivatives may include proteins comprising purification tags (e.g., poly His), human or humanized (e.g., humanized antibodies) proteins. Proteins or fragments thereof may be hybridized, cross-linked, or genetically engineered to comprise another protein, another chemical agent, or at least the active element of another protein or another chemical agent. For example, a protein, an antibody or a chemical agent may be hybridize, crosslinked or bonded with a second protein such, a second antibody, or a second chemical agent. Where 2 antibodies are linked, the antibodies may have the same or different binding specificity and may bind to the same or different epitopes on the same or different antigens. Another chemical agent, as used in this paragraph, may refer to a chemical agent (also called chemical compound) which may have an effect on viral infection such as, for example, nelfinavir mesylate. Non-limited examples of agents targeting viruses, such as the SARS-CoV-2, are listed below.

TABLE 1
AGENTS TARGETING VIRUSES INCLUDING SARS-COV-2

| Agents: Proteins (e.g.. | |
|---|---|
| Antibodies) and | |
| Chemicals | Examples |
| Antibodies to the NTD | 4A8 (Chi et al., Science (2020) Vol. 369, Issue |
| region of S protein of | 6504, pp. 650-655) |
| SARS-CoV-2 | |
| Antibodies to the HR1 | EK1C4 (Zhu Y, Yu D, Yan H, Chong H, He Y. |
| region of S protein of | Design of potent membrane fusion inhibitors |
| SARS-CoV-2 | against SARS-CoV-2, an emerging coronavirus |
| | with high fusogenic activity. J Virol. 2020; |
| | 94:e00635-20.) |
| Antibodies to the RBD | 47D11*; |
| region of S protein of | n3130*; |
| SARS-CoV-2 | n3088*; |
| | S309*; |
| | P2C-1F11*; |
| | P2B-2F6*; |
| | CR3022 (Tian X, Li C, Huang A, Xia S, Lu S, |
| | Shi Z, et al. Potent binding of 2019 novel |
| | coronavirus spike protein by a SARS |
| | coronavirus-specific human monoclonal |
| | antibody. Emerg Microbes Infect. 2020; |
| | 9:382-5.); |
| | B38*; |
| | H4*; |
| | 311mab-31B5*; |
| | 311mab-32D4*. |
| SARS-CoV monoclonal | 80R (Lan J, Ge J, Yu J, Shan S, Zhou H, Fan S, |
| antibodies | et al. Structure of the SARS-CoV-2 spike |
| | receptor-binding domain bound to the ACE2 |
| | receptor. Nature. 2020; 581:215-20.); |
| | m396 (Wang Q, Zhang Y, Wu L, Niu S, Song |
| | C, Zhang Z, et al. Structural and functional |
| | basis of SARS-CoV-2 entry by using human |
| | ACE2. Cell. 2020; 181:894-904.e9.); |
| | 201 (J Infect Dis. 2005 Feb. 15; 191(4):507-14. |
| | doi: 10.1086/427242. Epub 2005 Jan. 14.) |
| | 47D11; |
| | CR3014 (Meulen et al., PLoS Med . 2006 July; |
| | 3(7):e237. doi: |
| | 10.1371/journal.pmed.0030237.); |
| | CR3022 (Tian X, Li C, Huang A, Xia S, Lu S, |
| | Shi Z, et al. Potent binding of 2019 novel |
| | coronavirus spike protein by a SARS |
| | coronavirus-specific human monoclonal |
| | antibody. Emerg Microbes Infect. 2020; 9:382- |
| | 5.); |
| | 1A9 (Zheng et al., Euro Surveill. 2020 Jul. 16; |
| | 25(28):2000291); |
| | S230(Walls A C, Park Y J, Tortorici M A, Wall |
| | A, McGuire A T, Veesler D. Structure, |
| | function, and antigenicity of the SARS-CoV-2 |
| | spike glycoprotein. Cell. 2020; 181:281-92 |
| | e286.). |
| MERS-CoV | MERS-4 (Zhou et al., Nature Communications |
| monoclonal antibodies | volume 10, Article number: 3068 (2019)); |
| | MERS-27 (Zhou et al., Nature Communications |
| | volume 10, Article number: 3068 (2019)); |
| | 4C2 (Zhou et al., Nature Communications |
| | volume 10, Article number: 3068 (2019)); |
| | D12 (Zhou et al., Nature Communications |
| | volume 10, Article number: 3068 (2019)); |
| | m336 (Zhou et al., Nature Communications |
| | volume 10, Article number: 3068 (2019)); |
| SARS-CoV-2 | 4A8(Chi et al., Science (2020) Vol. 369, Issue |
| monoclonal antibodies | 6504, pp. 650-655); |
| | 47D11*; |
| | n3130*; |
| | n3088*; |
| | S309*; |
| | 311mab-31B5*; |
| | 311mab-32D4*; |
| | P2C-1F11*; |
| | P2B-2F6*; |
| | B38*; |
| | H4*; |
| | Casirivimab; |
| | imdevimab. |
| Peptides and Proteins | SARS-CoV-2 S HR2P fragment (aal 168-1203) |
| | (Xia S, Zhu Y, Liu M, Lan Q, Xu W, Wu Y, et |
| | al. Fusion mechanism of 2019-nCoV and fusion |
| | inhibitors targeting HR1 domain in spike |
| | protein. Cell Mol Immunol. 2020; 17:765-7.); |
| | EKI (Xia S, Yan L, Xu W, Agrawal A S, |
| | Algaissi A, Tseng C K, et al. A pan-coronavirus |
| | fusion inhibitor targeting the HR1 domain of |
| | human coronavirus spike. Sci Adv. 2019; |
| | 5:eaav4580); |
| | EK1C4 (Zhu Y, Yu D, Yan H, Chong H, He Y. |
| | Design of potent membrane fusion inhibitors |
| | against SARS-CoV-2, an emerging coronavirus |
| | with high fusogenic activity. J Virol. 2020; |
| | 94:e00635-20.) |
| | IBP02 (Zhu Y, Yu D, Yan H, Chong H, He Y. |
| | Design of potent membrane fusion inhibitors |
| | against SARS-CoV-2, an emerging coronavirus |
| | with high fusogenic activity. J Virol. 2020; |
| | 94:e00635-20.) |
| | Q-Griffithsin (Q-GRFT) (M78Q) (Dezzutti, C. |
| | S. et al. Development of HIV-1 rectal-specific |
| | microbicides and colonic tissue evaluation. |
| | PLoS One 9, e102585), Griffithsin (O’Keefe, B. |
| | R. et al. Scaleable manufacture of HIV-1 entry |
| | inhibitor griffithsin and validation of its safety |
| | and efficacy as a topical microbicide |
| | component. Proc Natl Acad Sci USA 106, |
| | 6099-6104). |
| Protease inhibitors | Viracept—(nelfinavir mesylate). |
| PIKfyve inhibitors | Apilimod; |
| | YM201636; |
| | Tetrandrine (also a TPC2 inhibitor). |
| TMPRSS2 inhibitors | Camostat mesylate. |
| Cathepsin inhibitors | E-64D Cathepsin inhibitor. |
| Furin inhibitors | PI8 (serpin proteinase inhibitor 8); |
| | MI-701 (Petidomimetic furin inhibitor); |
| | α-1-PDX (alpha-1-PDX—classic furin |
| | inhibitor). |
| Antivirals | IFN-α; ALFERON; IFN-α1b; IFN-α2a; |
| | remdesivir; chloroquine; favipiravir; lopinavir- |
| | ritonavir (Aluvia). |

Note 1:
some agents belong to multiple categories.
*See, Wang et al., A human monoclonal antibody blocking SARS-CoV-2 infection. Nat Commun. 2020; 11:2251; Chen et al., Human monoclonal antibodies block the binding of SARS-CoV-2 spike protein to angiotensin converting enzyme 2 receptor. Cell Mol Immunol. 2020; 17:647-9; Wu et al., Identification of fully human single-domain antibodies against SARS-CoV-2. Cell Host Microbe. 2020; 27:891-8.e5. https://doi.org/10.1101/2020.03.30.015990; Pinto et al., Structural and functional analysis of a potent sarbecovirus neutralizing antibody. bioRxiv. 2020;2020.04.07.023903. https://doi.org/10.1101/2020.04.07.023903; Ju et al., Potent human neutralizing antibodies elicited by SARS-CoV-2 infection. bioRxiv. 2020; https://doi.org/10.1101/2020.03.21.990770; Chi et al., A potent neutralizing human antibody reveals the N-terminal domain of the Spike protein of SARS-CoV-2 as a site of vulnerability. bioRxiv. 2020. https://doi.org/10.1101/2020.05.08.083964.

4.2 Interferons

One optional component of the composition is interferon. As used herein, the term “interferon” (abbreviated “IFN”) refers collectively to type 1 and type 2 interferons and including deletion, insertion, or substitution variants thereof, biologically active fragments thereof, and allelic forms thereof. As used herein, interferon refers collectively to type 1 and type 2 interferons. Type 1 interferon includes interferons alpha, beta, omega and their subtypes. Human interferon alpha has at least 14 identified subtypes while interferon beta has 3 identified subtypes.

The interferon may be at least one selected from the group consisting of: interferon, interferon mixture, Alferon, alpha-interferon species, recombinant or natural interferon alpha, recombinant or natural interferon alpha 2a, recombinant or natural interferon beta, recombinant or natural interferon beta 1b, recombinant, and natural interferon gamma.

The interferon is optionally an alpha-interferon. One preferred alpha interferon is ALFERON N Injection® the only approved natural, multi-species, α-interferon available in the United States. It is the first natural source, multi-species interferon and is a consistent mixture of at least seven species of α-interferon. The interferon is preferably a natural cocktail of at least seven species of human α-interferon. In contrast, the other available α-interferons are single molecular species of α-interferon made in bacteria using DNA recombinant technology. These single molecular species of α-interferon also lack an important structural carbohydrate component because this glycosylation step is not performed during the bacterial process.

Unlike species of α-interferon produced by recombinant techniques, ALFERON N Injection® is produced by human white blood cells that are able to glycosylate the multiple α-interferon species. Reverse phase HPLC studies show that ALFERON N Injection® is a consistent mixture of at least seven species of alpha interferon (α2, α4, α7, α8, α10, α16 and α17). This natural-source interferon has unique antiviral properties distinguishing it from genetically engineered interferons. The high purity of ALFERON N Injection® and its advantage as a natural mixture of seven interferon species, some of which, like species 8b, have greater antiviral activities than other species, for example, species 2b, which is the only component of INTRON A®. The superior antiviral activities, for example, in the treatment of chronic hepatitis C virus (HCV) and HIV infection, and tolerability of ALFERON N Injection® compared to other available recombinant interferons, such as INTRON A® and ROFERON A®, have been reported. ALFERON N Injection® is available as an injectable solution containing 5,000,000 international units (IU) per ml.

The interferon may be interferon species purified as a mixture of at least seven species of alpha-interferon produced by human white blood cells. The seven species may be, for example, interferon alpha 2; interferon alpha 4; interferon alpha 7; interferon alpha 8; interferon alpha 10; interferon alpha 16; and interferon alpha 17.

For internal or any administration, the α-interferon may, for example, be formulated in conventional manner for oral, nasal or buccal administration. Formulations for oral administration include aqueous solutions, syrups, elixirs, powders, granules, tablets and capsules which typically contain conventional excipients such as binding agents, fillers, lubricants, disintegrants, wetting agents, suspending agents, emulsifying agents, preservatives, buffer salts, flavoring, coloring and/or sweetening agents. α-Interferon may be administered by any method of administration of this disclosure. Preferably administration is by a suitable route including oral, nasal, parenteral (including injection) or topical (including transdermal, buccal and sublingual). It will be appreciated that the preferred route will vary with the condition and age of the recipient, the nature and severity of the virus infection and chosen tdsRNA.

The recommended dosage of the components will depend on the clinical status of the patient and the experience of the clinician in treating similar conditions. As a general guideline, a dosage of ALFERON N Injection® utilized for systemic infections is 3 IU/pound to 10 million IU/pound (e.g., subcutaneous injection) three times weekly. Experience to date is with dosages above 3 IU/lb. of patient body weight. Oral α-interferon (ALFERON LDO®) has been administered as a liquid solution in the range of 500-10,000 IU/day and calculated on the basis of a 150-pound human this is from 3.3 to 66.0 IU/lb per day. In one preferred embodiment, beneficial results are obtained at dosage levels of α-interferon in excess of 450 IU, that is greater than 3 IU/pound body weight. A healthcare provider would be able, however, to determine the optimal dose and schedule of low dose oral α-interferon (or any interferon) to achieve a desired antiviral effect.

Carrier Or Vehicle

Suitable agents may include a suitable carrier or vehicle for intranasal mucosal delivery. As used herein, the term “carrier” refers to a pharmaceutically acceptable solid or liquid filler, diluent or encapsulating material. In one aspect, the carrier is a suitable carrier or vehicle for intranasal mucosal delivery including delivery to the air passages and to the lungs of a subject.

A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories may be found in the U.S. Pharmacopeia National Formulary, 1857-1859, (1990).

Some examples of the materials which can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols, such as propylene glycol, polyols such as glycerin, sorbitol, mannitol and polyethylene glycol, esters such as ethyl oleate and ethyl laurate, agar, buffering agents such as magnesium hydroxide and aluminum hydroxide, alginic acid, pyrogen-free water, isotonic saline, Ringer's solution, ethyl alcohol and phosphate buffer solutions, Tris buffer solution, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants which can be administered with tdsRNA include water-soluble antioxidants such as ascorbic acid, cysteine hydrochloride, sodium bisulfite, sodium metabisulfite, sodium sulfite and the like, oil-soluble antioxidants such as ascorbyl palmitate, butylated hydroxyanisole (BHA), butylated hydroxytoluene (BHT), lecithin, propyl gallate, alpha-tocopherol and the like, and metal-chelating agents such as citric acid, ethylenediamine tetraacetic acid (EDTA), sorbitol, tartaric acid, phosphoric acid and the like.

Absorption-Promoting Agents

Suitable agents may include any suitable absorption-promoting agents. The suitable absorption-promoting agents may be selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacyl methyl sulfoxide, azone, sodium lauryl sulfate, oleic acid, and the bile salts, may be employed to enhance mucosal penetration of the tdsRNA. In additional aspects, surfactants (e.g., polysorbates) are employed as adjunct compounds, processing agents, or formulation additives to enhance intranasal delivery of the tdsRNA.

Delivery-Enhancing Agents

As used herein, the term “delivery-enhancing agents” refers to any agents which enhance the release or solubility (e.g., from a formulation delivery vehicle), diffusion rate, penetration capacity and timing, uptake, residence time, stability, effective half-life, peak or sustained concentration levels, clearance and other desired intranasal delivery characteristics (e.g., as measured at the site of delivery, or at a selected target site of activity such as the bloodstream) of tdsRNA or other biologically active compound(s).

In one aspect, enhancement of intranasal delivery can thus occur by any of a variety of mechanisms, for example by increasing the diffusion, transport, persistence or stability of tdsRNA, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

Mucolytic or Mucus Clearing Agents,

In another embodiment, the present formulations may also comprise other suitable agents such as mucolytic and mucus-clearing agents. The term “mucolytic and mucus-clearing agents,” as used herein, refers to any agents which may serve to degrade, thin or clear mucus from intranasal mucosal surfaces to facilitate absorption of intranasally administered biotherapeutic agents including tdsRNA. Based on their mechanisms of action, mucolytic and mucus clearing agents can often be classified into the following groups: proteases (e.g., pronase, papain) that cleave the protein core of mucin glycoproteins, sulfhydryl compounds that split mucoprotein disulfide linkages, and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine. Other effective agents that reduce mucus viscosity or adhesion to enhance intranasal delivery according to the methods of the disclosure include, e.g., short-chain fatty acids, and mucolytic agents that work by chelation, such as N-acylcollagen peptides, bile acids, and saponins (the latter function in part by chelating Ca²⁺ and/or Mg²⁺ which play an important role in maintaining mucus layer structure).

Ciliostatic Agents,

In another embodiment, the present formulations may comprise ciliostatic agents. As used herein, the term “ciliostatic agents” refers to any agents which are capable of moving a layer of mucus along the mucosa to removing inhaled particles and microorganisms. For use within these aspects of the disclosure, the foregoing ciliostatic factors, either specific or indirect in their activity, are all candidates for successful employment as ciliostatic agents in appropriate amounts (depending on concentration, duration and mode of delivery) such that they yield a transient (i.e., reversible) reduction or cessation of mucociliary clearance at a mucosal site of administration to enhance delivery of tdsRNA and other biologically active agents without unacceptable adverse side effects.

Within more detailed aspects, a specific ciliostatic factor may be employed in a combined formulation or coordinate administration protocol with tdsRNA, and/or other biologically active agents disclosed herein. Various bacterial ciliostatic factors isolated and characterized in the literature may be employed within these embodiments of the disclosure. Ciliostatic factors from the bacterium Pseudomonas aeruginosa include a phenazine derivative, a pyo compound (2-alkyl-4-hydroxyquinolines), and a rhamnolipid (also known as a hemolysin).

Penetration or Permeation-Promoting Agent

In another embodiment, the intranasal mucosal therapeutic and prophylactic formulations of the present disclosure may be supplemented with any suitable penetration-promoting agent that facilitates absorption, diffusion, or penetration of tdsRNA across mucosal barriers. The penetration promoter may be any promoter that is pharmaceutically acceptable. Thus, another aspect relates to compositions comprising tdsRNA and one or more penetration-promoting agents selected from sodium salicylate and salicylic acid derivatives (acetyl salicylate, choline salicylate, salicylamide, etc.), amino acids and salts thereof (e.g., monoaminocarboxlic acids such as glycine, alanine, phenylalanine, proline, hydroxyproline, etc., hydroxyamino acids such as serine, acidic amino acids such as aspartic acid, glutamic acid, etc., and basic amino acids such as lysine, etc.—inclusive of their alkali metal or alkaline earth metal salts), and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts).

Also provided as penetration-promoting agents within the methods and compositions of the disclosure are substances which are generally used as emulsifiers (e.g., sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidones carboxylic acid esters, N-alkylpyrrolidones, proline acyl esters, and the like.

In another embodiment, the present formulation may also comprise other suitable agents such as nitric oxide donor agents. As used herein, the term “nitric oxide donor agents” refers to any suitable agents which are capable of releasing nitric oxide. The release of nitric oxide may have a vasodilating effect. A nitric oxide (NO) donor may be selected as a membrane penetration-enhancing agent to enhance mucosal delivery of tdsRNA, and other biologically active agents disclosed herein. Various NO donors are known in the art and are useful in effective concentrations within the methods and formulations of the disclosure. Exemplary NO donors include, but are not limited to, nitroglycerine, nitroprusside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Within the methods and compositions of the disclosure, an effective amount of a selected NO donor may be coordinately administered or combinatorically formulated with tdsRNA, and/or other biologically active agents disclosed herein, into or through the mucosal epithelium.

Non-limiting examples of other permeation enhancers useful in the instant disclosure are the simple long-chain esters that are Generally Recognized As Safe (GRAS) in the various pharmacopoeial compendia. These may include simple aliphatic, unsaturated or saturated (but preferably fully saturated) esters, which contain up to medium length chains. Non-limiting examples of such esters include isopropyl myristate, isopropyl palmitate, myristyl myristate, octyl palmitate, and the like. The enhancers are of a type that are suitable for use in a pharmaceutical composition. The artisan of ordinary skill will also appreciate that those materials that are incompatible with or irritating to mucous membranes should be avoided.

For nasal administration, the enhancer is present in the composition in a concentration effective to enhance penetration of the pharmaceutically active agent that is to be delivered through the nasal mucosa. Various considerations should be taken into account in determining the amount of enhancer to use. Such considerations include, for example, the amount of flux (rate of passage through the membrane) achieved and the stability and compatibility of the components in the formulations. The enhancer is generally used in an amount of about 0.001 to about 40 (w/w) % of the composition. Specific ranges include, about 0.01% to about 30 (w/w), about 0.1 to about 25% (w/w), about 1% to about 15% (w/w), about 5 to 10% (w/w). Alternatively, the amount of the enhancer may range from about 1.0 to about 3% (w/w) or about 10 to about 20% (w/w).

In forming an emulsion in which the water-insoluble enhancer is a normally solid material, the enhancer is dissolved in a suitable solvent. If the enhancer is a normally liquid material which is water-immiscible, a suitable solvent for the enhancer may or may not be used, as appropriate. In certain embodiments, the enhancer is dissolved, dispersed, suspended, or solubilized in suitable solvent(s) such as alcohols, oils, glycerol, ethylene glycol, propylene glycol, hexane, acetone, freon, water, other polar or non-polar solvents, or a mixture, which is then added to a composition comprising an effective amount of the desired antigen admixed with a pharmaceutical carrier. In some cases, when the enhancers are in the liquid form, a “neat” solution of enhancer can be directly incorporated in the antigen, pharmaceutical carrier, and enhancer mixture, in which the concentration of enhancer ranges from about 0.1% to about 50% (w/w).

Any of the above permeation enhancers are useful, especially in nasal administration.

Vasodilator Or Vasoconstrictor Agents,

In another embodiment, the present formulation may also comprise other suitable agents such as vasodilator agents. As used herein, the term “vasodilator agents” refers to any agents which are vasoactive. A vasodilator agent may function within the disclosure to modulate the structure and physiology of the submucosal vasculature, increasing the transport rate of tdsRNA, and other biologically active agents into or through the mucosal epithelium and/or to specific target tissues or compartments (e.g., the systemic circulation). Vasodilator agents for use within the disclosure typically cause submucosal blood vessel relaxation by either a decrease in cytoplasmic calcium, an increase in nitric oxide (NO) or by inhibiting myosin light chain kinase. They are generally divided into 9 classes: calcium antagonists, potassium channel openers, ACE inhibitors, angiotensin-II receptor antagonists, alpha-adrenergic and imidazole receptor antagonists, beta-1-adrenergic agonists, phosphodiesterase inhibitors, eicosanoids and NO donors. Within certain methods and compositions of the disclosure, a selected vasodilator agent may be coordinately administered (e.g., systemically or intranasally, simultaneously or in combinatorially effective temporal association) or combinatorially formulated with tdsRNA and other biologically active agent(s) in an amount effective to enhance the mucosal absorption of the active agent(s) to reach a target tissue or compartment in the subject.

In another embodiment, the present formulation may also comprise other suitable agents such as vasoconstrictor agents. As used herein, the term “vasoconstrictor agents” refers to any substances which may cause vasoconstriction. Vasoconstrictor agents may also be called vasoconstrictors, vasopressors, or simply “pressors.” Vasoconstrictor agents may usually cause an increase in systemic blood pressure, but when they are administered in specific tissues, localized blood flow may be reduced. The extent of vasoconstriction may be slight or severe depending on the substance of vasoconstrictor agents. Many vasoconstrictor agents may also cause pupil dilation. Vasoconstrictor agents may include any suitable substances such as antihistamines, decongestants and stimulants that are used to treat ADHD. Suitable vasoconstrictor agents have been previously described by Dhuria, Hanson, et al. (Dhuria, Hanson, et al., 2009).

RNase Inhibitory Agent and Enzyme Inhibitor

In some embodiments, for example, nasal vaccines, the disclosure encompasses the delivery of a protein, peptide or other nucleic acid in addition to tdsRNA. Therefore, the compositions of the present disclosure may contain an enzyme inhibitor. As is well known to practitioners in nucleic acid, peptide and protein biochemistry, these biopolymers tend to be very sensitive to the presence of enzymes, such as RNase and proteolytic enzymes, that rapidly degrade the biopolymer when present in even minute amounts. Typical enzyme inhibitors that are commonly employed and that may be incorporated into the present disclosure include, but are not limited to leupeptin, aprotinin, and the like. Enzyme inhibitors also include nuclease inhibitors such as DNase inhibitors and RNase inhibitors. RNase inhibitors are commonly used as a precautionary measure in enzymatic manipulations of RNA to inhibit and control RNase. These are commercially available from a number of sources such as, for example, Invitrogen (SUPERase, In RNase Inhibitor, RNaseOUT, RNAsecure, and RNase Inhibitor).

Selective Transport-Enhancing Agents

In another embodiment, the present formulation may also comprise other suitable agents such as selective transport-enhancing agents. As used herein, the term “selective transport-enhancing agent” refers to any agent that facilitates transport of tdsRNA and/or one or more biologically active agents including vaccines. The compositions and delivery methods of the disclosure may optionally incorporate a selective transport-enhancing agent that facilitates transport of one or more biologically active agents. These transport-enhancing agents may be employed in a combinatorial formulation or coordinate administration protocol with tdsRNA disclosed herein, to coordinately enhance delivery of one or more additional biologically active agent(s). Alternatively, the transport-enhancing agents may be employed in a combinatorial formulation or coordinate administration protocol to directly enhance mucosal delivery of tdsRNA, with or without enhanced delivery of an additional biologically active agent.

Exemplary selective transport-enhancing agents for use within this aspect of the disclosure may include, but are not limited to, glycosides, sugar-containing molecules, and binding agents such as lectin binding agents, and stabilizers. For example, specific “bioadhesive” ligands, including various plant and bacterial lectins, which bind to cell surface sugar moieties by receptor-mediated interactions can be employed as carriers or conjugated transport mediators for enhancing mucosal, e.g., nasal delivery of biologically active agents within the disclosure. Certain bioadhesive ligands for use within the disclosure will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more tdsRNA or functionally equivalent fragment proteins, analogs and mimetics, and other biologically active agent(s) into and/or through mucosal epithelia. These and other selective transport-enhancing agents significantly enhance mucosal delivery of macromolecular biopharmaceuticals (particularly peptides, proteins, oligonucleotides and polynucleotide vectors) within the disclosure.

Additional intranasal mucosal delivery-enhancing agents that are useful within the coordinated administration and processing methods and combinatorial formulations of the disclosure may also include, but are not limited to, mixed micelles, enamines, nitric oxide donors (e.g., S-nitroso-N-acetyl-DL-penicillamine, NOR1, NOR4—which are preferably co-administered with an nitric oxide scavenger such as carboxy-PITO or diclofenac sodium), sodium salicylate, glycerol esters of acetoacetic acid (e.g., glyceryl-1,3-diacetoacetate or 1,2-isopropylideneglycerine-3-acetoacetate), and other release-diffusion or intra- or trans-epithelial penetration-promoting agents that are physiologically compatible for intranasal mucosal delivery. Other absorption-promoting agents may be selected from a variety of carriers, bases and excipients that enhance mucosal delivery, stability, activity or trans-epithelial penetration of the tdsRNA. These include, inter alia, cyclodextrins and beta-cyclodextrin derivatives (e.g., 2-hydroxypropyl-beta-cyclodextrin and heptakis(2,6-di-O-methyl-beta-cyclodextrin). These compounds, optionally conjugated with one or more of the active ingredients and further optionally formulated in an oleaginous base, enhance bioavailability in the intranasal mucosal formulations. Yet additional absorption-enhancing agents adapted for intranasal mucosal delivery may also include medium-chain fatty acids, including mono- and diglycerides (e.g., sodium caprate—extracts of coconut oil, CAPMUL), and triglycerides (e.g., amylodextrin, Estaram 299, Miglyol 810).

Stabilizing Delivery Vehicle, Carrier, Support or Complex-Forming Species

In another embodiment, the present formulation may also comprise other suitable agents such as a stabilizing delivery vehicle, carrier, support or complex-forming species. The coordinate administration methods and combinatorial formulations of the instant disclosure may optionally incorporate effective lipid or fatty acid-based carriers, processing agents, or delivery vehicles, to provide improved formulations for mucosal delivery of tdsRNA or functionally equivalent fragment proteins, analogs and mimetics, and other biologically active agents. For example, formulations and methods for mucosal delivery can comprise one or more of these active agents, such as a peptide or protein, admixed or encapsulated by, or coordinately administered with, a liposome, mixed micellar carrier, or emulsion, to enhance chemical and physical stability and increase the half-life of the biologically active agents (e.g., by reducing susceptibility to proteolysis, chemical modification and/or denaturation) upon mucosal delivery.

Within certain aspects of the disclosure, specialized delivery systems for biologically active agents may comprise small lipid vesicles known as liposomes or micelles. These are typically made from natural, biodegradable, non-toxic, and non-immunogenic lipid molecules, and can efficiently entrap or bind drug molecules, including peptides and proteins, into, or onto, their membranes. The attractiveness of liposomes as a nucleic acid delivery system is increased by the fact that the encapsulated tdsRNA can remain in their preferred aqueous environment within the vesicles, while the liposomal membrane protects them against nuclease and other destabilizing factors.

Additional delivery vehicles carrier, support or complex-forming species for use within the disclosure may include long and medium-chain fatty acids, as well as surfactant mixed micelles with fatty acids. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers.

For use within the methods of the disclosure, long-chain fatty acids, especially fusogenic lipids (unsaturated fatty acids and monoglycerides such as oleic acid, linoleic acid, linoleic acid, monoolein, etc.) provide useful carriers to enhance mucosal delivery of tdsRNA, and other biologically active agents disclosed herein. Medium-chain fatty acids (C6 to C12) and monoglycerides have also been shown to have enhancing activity in intestinal drug absorption and can be adapted for use within the mucosal delivery formulations and methods of the disclosure. In addition, sodium salts of medium and long-chain fatty acids are effective delivery vehicles and absorption-enhancing agents for mucosal delivery of biologically active agents. Thus, fatty acids can be employed in soluble forms of sodium salts or by the addition of non-toxic surfactants, e.g., polyoxyethylated hydrogenated castor oil, sodium taurocholate, etc. Other fatty acid and mixed micellar preparations that are useful within the disclosure include, but are not limited to, Na caprylate (C8), Na caprate (C10), Na laurate (C12) or Na oleate (C18), optionally combined with bile salts, such as glycocholate and taurocholate.

### 4: Administration

Administration Methods

Any compound, formulation, or pharmaceutical composition in this disclosure may be administered by any of the administration methods disclosed or any local or systemic route known in the art including enteral (e.g., oral, feeding tube, enema), topical (e.g., devices such as a nebulizer for inhalation through the respiratory system, skin patch acting epicutaneously or transdermally, suppository acting in the rectum or vagina), and parenteral (e.g., subcutaneous, intravenous, intramuscular, intradermal, or intraperitoneal injection, buccal, sublingual, or transmucosal, inhalation or instillation intranasally or intratracheally).

In some embodiments, the tdsRNA is administered continuously. In some embodiments, the tdsRNA is administered intermittently.

The pharmaceutical composition and/or the active agents including tdsRNA for administration may be micronized by milling or grinding solid material, dissolved in a vehicle (e.g., sterile buffered saline or water) for injection or instillation (e.g., spray), topically applied, or encapsulated in a liposome or other carrier for targeted delivery. The preferred administration route may vary with the age, condition, gender, or health status of the subject, the nature of the disease or other pathological condition, including the number and severity of symptoms, and the chosen active ingredient.

Suitable effective treatment protocols include, for example, administering to a subject a therapeutically or prophylactically effective amount of a tdsRNA, preferably via at least one local or systemic route and/or by using at least one mode of administration or device as described above.

In some embodiments, a combination treatment of the present disclosure comprises administration of tdsRNA and one or more antiviral agents (e.g., interferon, cyclophilin inhibitor such as cyclosporine A, nucleoside analog such as ribavirin, protease inhibitor such as lopinavir or ritonavir, antibody specific for SARS-CoV-2) to an infected subject. The combination treatment may be administered in any suitable manner known in the art. For example, the tdsRNA may be administered sequentially (at different times) or concurrently (at the same time) with the one or more antiviral agents. Also, tdsRNA and one or more antiviral agents (separately or together) may be administered prophylactically (i.e., before infection) or at early-onset (i.e., soon after infection).

Routes and Modes of Administration

The pharmaceutical composition comprising tdsRNA may be administered to a subject by any local or systemic route known in the art including enteral (e.g., oral, feeding tube, enema), topical (e.g., devices such as a nebulizer for inhalation through the respiratory system, skin patch acting epicutaneously or transdermally, suppository acting in the rectum or vagina), and parenteral (e.g., subcutaneous, intravenous, intramuscular, intradermal, or intraperitoneal injection; buccal, sublingual, or transmucosal; inhalation or instillation intranasally or intratracheally). The pharmaceutical composition and/or the active agents may be micronized by milling or grinding solid material, dissolved in a vehicle (e.g., sterile buffered saline or water) for injection or instillation (e.g., spray), topically applied, or encapsulated in a liposome or other carrier for targeted delivery. It will be appreciated that the preferred route may vary with the age, condition, gender, or health status of the subject; the nature of the disease or other pathological condition, including the number and severity of symptoms; and the chosen active ingredient.

Suitable effective treatment protocols include, for example, administering to a subject a therapeutically or prophylactically effective amount of a tdsRNA, preferably via at least one local or systemic route and/or by using at least one mode of administration or device as described above.

In some embodiments, a combination treatment of the present disclosure comprises administration of tdsRNA and one or more antiviral agents (e.g., interferon, cyclophilin inhibitor such as cyclosporine A, nucleoside analog such as ribavirin, protease inhibitor such as lopinavir or ritonavir, antibody specific for SARS-CoV-2) to an infected subject. Any compound or formulation in this disclosure may be administered by any of the administration methods disclosed. The tdsRNA may be administered in any suitable manner known in the art. For example, the tdsRNA may be administered sequentially (at different times) or concurrently (at the same time) with the one or more antiviral agents. Instead, tdsRNA and one or more antiviral agents (separately or together) may be administered prophylactically (i.e., before infection) or at early-onset (i.e., soon after infection).

In some embodiments, the tdsRNA is administered continuously. In some embodiments, the tdsRNA is administered intermittently.

Nasal Administration Definition

Nasal administration and “Pulmonary Airway Administration” have the same meaning in this disclosure and they refer to, at least, any administration method to any part of an airway (pulmonary airway) of a subject. The airway includes any parts that are involved with breathing through the mouth.

Nasal Administration's Reach

The pulmonary airway comprises those parts of the respiratory system through which air flows, conceptually beginning (on inhalation from the external environment) at the nose and mouth, and terminating in the alveoli. From the mouth or nose, inhaled air passes through the pharynx into the trachea, where it separates into the left and right main bronchi at the carina, situated at the level of the second thoracic vertebra. The main bronchi then branch into large bronchioles, one for each lobe of the lung. Within the lobes, the bronchioles further subdivide some twenty times, ending in clusters of alveoli. Therefore, non-limiting examples of nasal administration encompass administration to at least the following parts of a body: mouth, nose, pharynx, trachea, sinus, bronchi, carina, large bronchioles, lung, lobes of the lung, alveoli, and lung epithelial lining to the bloodstream.

Cells that can be Contacted by Nasal Administration

The cells that may be contacted by nasal administration include any cells in a mammalian (e.g., human) conducting airway such as any cell of the patient's respiratory system including any cells of the following parts of the body: mouth, nose, pharynx, trachea, sinus, bronchi, carina, large bronchioles, lung, lobes of the lung, alveoli, and lung epithelial lining to the bloodstream. These cells include epithelium cells. The epithelium cells include ciliated cells, goblet cells, non-ciliated cells, basal cells. In certain embodiments, the airway epithelial cell is a lung cell, a nasal cell, a tracheal cell, a bronchial cell, a bronchiolar or alveolar epithelial cell. In certain embodiments, the airway epithelial cells are present in a mammal.

### 5: Formulations and Dosage

Formulation

Formulations for administration (i.e., pharmaceutical compositions) may include pharmaceutically acceptable carrier with the tdsRNA.

Pharmaceutical carriers include suitable non-toxic vehicles in which a composition of the disclosure is dissolved, dispersed, impregnated, or suspended, such as water or other solvents, fatty materials, celluloses and their derivatives, proteins and their derivatives, collagens, gelatine, polymers, adhesives, sponges, fabrics, and the like and excipients which are added to provide better solubility or dispersion of the drug in the vehicle. Such excipients may include non-toxic surfactants, solubilizers, emulsifiers, chelating agents, binding materials, lubricants softening agents, and the like. Pharmaceutically acceptable carriers may be, for example, aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

A liquid carrier may be present in the composition in a concentration effective to serve as a suitable vehicle for the compositions of the present disclosure. In general, the carrier is used in an amount of about 40 to about 98 wt. %, or about 50 to about 98 wt. % of the composition. The compositions of the present disclosure are preferably delivered as nasal sprays.

The liquid carrier may be water or any other suitable liquid, solvent, or mixture thereof. An antigen may be dispersed or dissolved in the liquid carrier in a therapeutically effective amount. The water may contain suitable buffering agents to result in a pH wherein the particular antigen is delivered optimally, or it may contain other carriers, such as glycerin, propylene glycol, polyethylene glycols of various sizes, amino acid modifiers, such as arginine and the like, and other suitable soluble excipients, as is known to those who are proficient in the art of compounding or pharmaceutics.

The preferred formulation may vary with the age, condition, gender, or health status of the subject, the nature of the disease or other pathological condition, including the number and severity of symptoms, and the chosen active ingredient.

The tdsRNA in solid form may be dissolved using known diluents for administration such as, for example, physiological phosphate-buffered saline, and then infused intravenously. The tdsRNA may be a combination or any subset of dsRNA described above. It is understood that in one aspect, tdsRNA may comprise a combination of all of the examples of tdsRNA described above or any subset of the above examples. With respect to the subsets, the specific exclusion of one or more specific embodiment of tdsRNA is also envisioned. As non-limiting examples, tdsRNA may comprise any of the following: (1) all of the examples of tdsRNA as described above, (2) all of the examples of tdsRNA described above but without rIₙ.r(C₁₁₋₁₄U)ₙ, (3) Rugged dsRNA, (4) rIₙ.r(C₁₂U)ₙ, (5) tdsRNA as described above but without rIₙ.r(C₁₁₋₁₄U)ₙ and without Rugged dsRNA.

The composition of the present disclosure may exist in various forms, for example, an oil-in-water emulsion, a water-in-oil emulsion, and a water-in-oil-in-water emulsion. The active compounds of the present disclosure, including the embodiments where tdsRNA is in combination with other agents, may exist in either the continuous or the dispersed phase or in both phases depending upon whether the compounds are hydrophilic, lipophilic, or amphiphilic. As an example, the emulsion comprises oil droplets dispersed in a continuous aqueous phase with a lipophilic enhancer being contained in the oil droplets and a water-soluble pharmaceutically-active compound dissolved in the continuous aqueous phase. In a preferred embodiment wherein an oil phase is utilized, the concentration of the oil in the oil phase is such that it does not promote crystallization.

The composition of the present disclosure may also comprise an emulsifying agent for use in aiding the formation of an emulsion. Essentially any suitable hydrocolloid emulsifying agent, typically a solid material, or a mixture of two or more such emulsifying agents can be used in the practice of the present disclosure. Hydrocolloid emulsifying agents include: vegetable derivatives, for example, acacia, tragacanth, agar, pectin, and carrageenan; animal derivatives, for example, gelatin, lanolin, cholesterol, and lecithin; semi-synthetic agents, for example, methylcellulose and carboxymethylcellulose; and synthetic agents, for example, acrylic emulsifying agents such as carbomers. The hydrocolloid emulsifying agent forms hydrocolloids (hydrated lyophilic colloids) around the emulsified liquid droplets of the emulsion. The hydrocolloid serves as a protective layer around each emulsified droplet which physically repulses other droplets, thus hindering Ostwald ripening (the tendency of emulsified droplets to aggregate).

In contrast, other emulsifying agents typically protect the emulsified droplets by forming a liquid crystalline layer around the emulsified droplets. In compositions which employ a liquid crystalline layer-forming emulsifying agent, the hydrophilic-lipophilic balance (HLB) of the oil phase of the emulsion must be matched with that of the emulsifying agent to form a stable emulsion and, often, one or more additional emulsifying agents (secondary emulsifying agents) must be added to further stabilize the emulsion. The aforementioned liquid crystalline layer also retards the release of the compounds of the dispersed phase upon contact with the target substrate.

The hydrocolloid emulsifying agents for use in the composition of the present disclosure include compounds which exhibit a low level of irritability or no irritability to the target membrane and which have good bioadhesive and mucoadhesive properties. Examples of hydrocolloid emulsifying agents which exhibit such properties include cellulosic emulsifying agents and acrylic emulsifying agents, including, for example, those which have an alkyl group containing from about 10 to about 50 carbon atoms. Particularly preferred acrylic emulsifying agents for use in the present disclosure are copolymers of a carboxylic acid and an acrylic ester (described, for example, in U.S. Pat. No. 3,915,921 to Schlatzer and U.S. Pat. No. 4,509,949 to Huang et al.), with those which are cross-linked being especially preferred.

The emulsifying agent is present in the composition in a concentration that is effective to form the desired liquid emulsion. In general the emulsifying agent is used in an amount of about 0.001 to about 5 wt. % of the composition, and more generally in an amount of about 0.01 to about 5 wt. % of the composition, and most generally in an amount of about 0.1 to about 2 wt. % of the composition.

The composition of the present disclosure may include, as an optional ingredient, particulate solids dispersed in the composition. For example, the composition may include an additional pharmaceutically-active compound dispersed in the liquid continuous phase of the emulsion in the form of microcrystalline solids or nanoparticulates.

The liquid compositions are particularly suited for nasal administration.

Nasal Compositions

In one embodiment, a composition for enhancing intranasal delivery includes a combination of tdsRNA and active compounds prepared for nasal delivery. The combination of tdsRNA and active compounds may be applied in a subsequent manner or a simultaneous manner. In a preferred embodiment, the mixture will be in the form of an aqueous solution. In other embodiments, the mixture will be a powder or a dried, powdered, or lyophilized form of the mixture. In some embodiments, these forms will be re-hydrated before delivery.

Nasal Formulations

In one aspect, the present disclosure relates to formulations for nasal delivery of tdsRNA. In one aspect, tdsRNA is the sole active compound and may be free of any other active compounds. In another aspect, the tdsRNA may be co-administered with one or more additional active compounds.

Each of the agents and chemicals described herein, including any combinations thereof, may be added to a tdsRNA for administration, including nasal administration, to a subject.

Medicament

In another aspect, a medicament (e.g., a pharmaceutical composition) containing the tdsRNA is provided. Optional other components of the medicament include excipients and a vehicle (e.g., aqueous buffer or water for injection) packaged aseptically in one or more separate containers (e.g., nasal applicator or injection vial). Further aspects will be apparent from the disclosure and claims herein.

Dosage for any Form of Administering

For a subject (e.g., 150 lb or 70 Kg human) the dose of dsRNA may range from 0.1 to 1,000,000 μg, preferably from 0.4 to 400,000 μg. If desired, the dosage may be scaled to other subjects of a different mass. For example, tdsRNA may be dosed at from about 0.5 mg to about 60 mg per day, from about 5 mg to about 400 mg per day, from about 25 mg to about 700 mg per day, or from about 10 mg to about 800 mg per day in a subject. As another example, the administration may be in 50-1400 milligrams every other day leading to an average daily dosage of 25-700 milligrams per day.

In certain embodiments, the tdsRNA is administered in a dose of 0.1 mg/kg, 0.2 mg/kg, 0.3 mg/kg, 0.5 mg/kg, 0.7 mg/kg, 0.8 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg or 20 mg/kg.

In another embodiment, the dosage of a tdsRNA of the present disclosure is a unit dose of about 0.1-20 mg/kg, about 0.1-10 mg/kg, about 0.1-8 mg/kg, about 0.1-7 mg/kg, about 0.1-6 mg/kg, about 0.1-5 mg/kg, about 0.1-4 mg/kg, about 0.1-3 mg/kg, about 0.2-3 mg/kg, about 0.3-3 mg/kg, about 0.4-3 mg/kg, about 0.6-3 mg/kg, about 0.8-3 mg/kg, about 0.1-2 mg/kg, about 0.1-1 mg/kg.

Total daily dose may vary from 20 mg to 200 mg, 50 mg to 150 mg, 80 mg to 140 mg.

In a preferred embodiment, a tdsRNA is administered at a unit dose of about 0.1 mg/kg, about 0.2 mg/kg, about 0.4 mg/kg, about 0.6 mg/kg, about 0.8 mg/kg, about 1 mg/kg, about 2 mg/kg, about 3 mg/kg, about 4 mg/kg or about 5 mg/kg.

In one embodiment, the tdsRNA is administered at a dose from about 1 mg/kg to 10 mg/kg biweekly.

In certain embodiments, the tdsRNA is administered one dose per day, one dose every 2 days, one dose every 3 days, one dose every 4 days, one dose every 5 days, once a week, once every two weeks, or once every four weeks, preferably one dose every 3 days.

In certain embodiments, the tdsRNA is administered as a single dose, in two doses, in three doses, in four doses, in five doses, or in 6 or more doses.

The dosing schedule can vary from, e.g., once a week to once every 2 weeks, once a week to once every 3 weeks, once a week to once every 4 weeks. In one embodiment, the tdsRNA is administered at a dose from about 0.50 mg/kg to 10 mg/kg every other week.

In certain embodiments, the dose frequency may vary from once a day to once a month.

The recommended dosage of tdsRNA will depend on the clinical status of the subject and the physician's or veterinarian's experience treating the disease or other pathological condition. tdsRNA may be dosed at from about 0.5 mg to about 60 mg per day, from about 5 mg to about 400 mg per day, from 25 mg to about 700 mg per day, or from about 10 mg to about 800 mg per day in a subject (e.g., body mass of about 70-80 Kg for a human patient) on a schedule of either once a day up to 7 days weekly or once-weekly to thrice-weekly (preferably twice weekly), albeit the dose amount and/or frequency may be varied by the physician or veterinarian in response to the subject's symptoms. That is, for example, the administration may be in 50-1400 milligrams every other day leading to an average daily dosage of 25-700 milligrams per day.

A dosing period is usually about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, and, in one embodiment, 6, 7, 8, 9, 10, 11, 12, 13, or 14 days, for example, 7 or 14 days.

In certain embodiments, multiple (for example, 2, 3, 4, 5, 6, 7, 8, 9, 10 or more) doses of a tdsRNA are administered to a subject in need of treatment.

In any dosage, and particularly preferred for nasal administration, tdsRNA may be administered at a dose of 5 μg to 10 μg; 10 μg to 20 μg; 20 μg to 50 μg; 50 μg to 100 μg; 100 μg to 200 μg; 200 μg to 500 μg; 500 μg to 1000 μg; 1000 μg to 1500 μg; 1500 μg to 2000 μg; or any combination thereof.

Unless otherwise specified, “composition,” “a composition,” or “the composition” includes, at least, a composition of the disclosure or includes at least tdsRNA and optionally a vaccine. Compositions may be optionally filtered and sterilized to enhance safety, stability and solubility. The composition may be formulated to enhance the delivery method. For example, the formulation may be formulated to enhance intraperitoneal delivery or nasal delivery.

### 6: Compositions and Methods that are Generally Applicable and Particularly Applicable for Nasal Administration

Compositions (Nasal Formulations) Preferred for Nasal Administration

Unless otherwise specified, “composition,” “a composition,” or “the composition” includes, at least, a composition of the disclosure or includes at least tdsRNA. Compositions may be optionally filtered and sterilized to enhance safety, stability and solubility.

In one embodiment, a composition for enhancing intranasal delivery includes tdsRNA and optionally active compounds prepared for nasal delivery. The combination of tdsRNA and active compounds may be applied in a subsequent (sequential) manner or a simultaneous (parallel) manner. In a preferred embodiment, the mixture will be in the form of an aqueous solution. In other embodiments, the mixture will be a powder or a dried, powdered, or lyophilized form of the mixture. In some embodiments, these forms will be re-hydrated before delivery. The composition may be in solid, liquid or any other form such as gels and liposomes.

A composition of the disclosure (e.g., tdsRNA) that is used in nasal administration is considered a nasal composition. Compositions of the disclosure are not limited to nasal administration. That is, any composition of the disclosure may be used as a nasal composition. Similarly, nasal compositions may be used for any other purposes such as non-nasal administration.

Simultaneous administration (also called parallel administration) may also comprise administration of two or more compositions at the same time. For example, two or more separate nasal nozzles and sprayers can each dispense a different composition for simultaneous administration. Simultaneous administration may also dispense compositions of different forms. For example, a dry powder and a liquid may be dispensed together in separate sprayers at the same time.

Each of the agents and chemicals described herein, including any combinations thereof, may be administered together with a composition of the disclosure (e.g., tdsRNA), nasally or otherwise, to a subject. Non-limiting examples of other compounds for nasal administration include RNA, DNA, adjuvants, proteins, interferons, pathogens (intact, inactivated, attenuated) or parts thereof. Pathogens may be used as a vaccine. Non-limiting examples of pathogens include at least viruses, bacteria, yeast, fungi, and the like. “Parts thereof” of a pathogen may be useful as a vaccine. Non-limiting examples of these parts would include, at least, unpurified, semi-purified and purified parts. Pathogen, and especially parts thereof, may be collected from at least one selected from the group consisting of a pathogen, a pathogen culture grown in a laboratory (in vitro), a pathogen in an animal, a pathogen collected from the wild (e.g., from a diseased animal), a cloned or and genetically engineered pathogen, an in vitro synthesized pathogen (e.g., cell free in vitro synthesis), a synthetic pathogen (e.g., from a peptide synthesizer), from a transgenic organism (e.g., transgenic mammal, yeast, bacteria or the like).

As discussed, the pathogen of the previous sentence includes “parts thereof.” Non-limiting examples of these parts include at least one selected from the group consisting of protein including recombinant protein, nucleic acid including DNA, RNA, synthetic nucleic acid, and combinations thereof (e.g., combinations of synthetic and natural nucleic acid in a double strand), antigens, peptides.

Preferred embodiments of compounds for administration include tdsRNA, influenza virus or parts thereof including inactivated or attenuated forms and antigens thereof, coronavirus or parts thereof including inactivated or attenuated forms and antigens thereof.

We note that tdsRNA is stable as a solid or dissolved in water and therefore any additional component is optional. Other components may benefit from additional ingredients described herein.

In certain embodiments, the therapeutic agent is administered with an agent that disrupts, e.g., transiently disrupts, tight junctions, such as EGTA (see U.S. Pat. No. 6,855,549).

Furthermore, since nasal administration may be perceived by a sense of smell in the subject, additives that improve the fragrances or nasal acceptance or reduce irritation may be added. These include buffers and preservatives if the composition is not made sterile, for example, methyl hydroxybenzoate, antioxidants, flavoring agents, volatile oils, buffering agents and surfactants.

Specific Examples of Compositions

Aerosol compositions can be made with liquid and dried compositions of the disclosure to be administered via inhalation. These aerosol compositions can be placed into pressurized acceptable propellants, such as dichlorodifluoromethane, propane, and nitrogen. Compositions may be formulated as pharmaceuticals for non-pressured preparations, such as in a nebulizer or an atomizer. For compositions to be administered from multiple-dose containers, antimicrobial agents can be added.

Liquid solutions may be suitable for any administration including nasal administration. Liquid compositions may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. The composition of the disclosure can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The compositions may be formulated as dry, semidry, or liquid particles. The particulate pharmaceutical composition may optionally be combined with a carrier to aid in dispersion or transport. A suitable carrier such as a sugar (i.e., dextrose, lactose, sucrose, trehalose, mannitol) may be blended with the active compound or compounds in any suitable ratio.

Specific examples of compositions forms include at least the following: aerosol of liquid, aerosol suspension of respirable solid, dry powder inhalants, metered-dose inhalants, liquid/liquid suspensions, emulsions, suspensions, oil in water emulsion, and water in oil emulsions.

In reference to particles or droplets, it is envisioned that a particle or a droplet may be a solid, a liquid, or other types of particle such as a gel, a liposome, and the like. Also, it is envisioned that a composition may be dispensed as one type of particle but is delivered to a subject as a second type of particle. For example, a composition may be dispensed as a liquid particle with a high evaporation rate such that the liquid is transformed into a solid by the time the particle reaches the subject.

Certain devices require the use of various compositions suitable for the dispensing of some compositions of the present disclosure. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified systems may also be prepared in different compositions depending on the type of chemical modification or the type of device employed.

Compositions suitable for use with a nebulizer may also include a buffer and a simple sugar (e.g., for stabilization of the composition and regulation of osmotic pressure). The carrier is typically water (and most preferably sterile, pyrogen-free water) or a dilute aqueous alcoholic solution, preferably made isotonic, but may be hypertonic with body fluids by the addition of, for example, sodium chloride. The nebulizer composition may also contain a surfactant to reduce or prevent surface induced aggregation caused by atomization of the solution in forming the aerosol. Optional additives include preservatives if the composition is not made sterile, for example, methyl hydroxybenzoate, antioxidants, flavoring agents, volatile oils, buffering agents and surfactants.

Compositions for use with a metered-dose inhaler device may generally comprise a finely divided powder (a composition of the disclosure) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Compositions for dispensing from a powder inhaler device may comprise a finely divided dry powder containing a composition as described herein, and may also include a bulking agent, such as lactose, sorbitol, sucrose, or mannitol in amounts that facilitate dispersal of the powder from the device, e.g., 50 to 90% by weight of the composition. The composition may be prepared in particulate form with an average particle size of less than 10 mm (or microns), most preferably 0.5 to 5 mm, for most effective delivery to the distal lung.

Non-limiting specific examples of nasal (pulmonary) administration include at least one or more of the administration methods such as oral administration (through the mouth, by breathing through the mouth); intranasal administration (e.g., by nose drops); inhalation administration; aerosol administration; intra-airway (e.g., tracheal or bronchial) administration; bronchoscopic instillation; intratracheal administration; mucosal administration; dry powder administration; respiratory administration; instillation administration.

Another example of nasal administration includes any deposition to any part of the airway, including, for example, by spray, by a swab, intratracheal deposition, intrabronchial deposition and bronchoscopic deposition, nasal rinse, nasal lavage, a temporary or permanent depot implant.

Administration by “inhalation” may be performed using a composition of the disclosure of a size sufficiently small to pass through the mouth or nose and larynx, past the oropharyngeal region, upon inhalation and into the bronchi and alveoli of the lungs. In general, particles (droplets, liquid or solid) ranging from about 1 to 10 microns in size (more particularly, less than about 5 microns in size) are respirable and suitable for administration by inhalation. The particles can be solid or liquid. In some embodiments, such preparations have a mean particle size of 4, 5, 6, 7, 8, 9, 10, 11, 12, or 13 microns.

In some embodiments, preparations for inhaled or aerosol delivery are formulated as a dry powder. In some embodiments, preparations for inhaled or aerosol delivery are formulated as a wet powder, for example through inclusion of a wetting agent. in some embodiments, the wetting agent is selected from the group consisting of water, saline, or other liquid of physiological pH. In some embodiment, the particles may be a liquid.

Administration by intranasal administration may be performed by particles of a larger size formulated and delivered to topically treat the nasal epithelium. Particles or droplets used for intranasal administration generally have a diameter that is larger than those used for administration by inhalation. For intranasal administration, a particle size in the range of 10-500 microns is preferred to ensure retention in the nasal cavity.

In some embodiments, particles for inhalation and particles for intranasal administration may be administered together. That is, particles of 1 to 500 microns are used. In some embodiments, particles of 1-10 or 1-13 microns are selected for or enriched. In other embodiments, particles of 10-500 microns, or 15 to 500 micron are selected for or enriched.

The compositions of the disclosure may be administered as a plurality of drops to the nasal or buccal cavity. A dose may be, for example, 1-100, 1-50, 1-20, 1-10, 1-5, drops.

In some embodiments, inventive compositions are administered using a device that delivers a metered dosage of composition.

Aerosols of liquid particles of the compositions of the disclosure may be produced by any suitable means, such as with a nebulizer, pressure-driven jet nebulizer, an ultrasonic nebulizer, or other means.

Aerosols of solid particles comprising the composition of the disclosure may likewise be produced with any solid particulate therapeutic aerosol generator. One illustrative type of solid particulate aerosol generator is an insufflator. Suitable compositions for administration by insufflation include finely comminuted powders which may be delivered by means of an insufflator or taken into the nasal cavity in the manner of a snuff. In the insufflator, the powder (e.g., a metered-dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the composition of the disclosure or of a powder blend comprising the composition and a suitable powder diluent, such as lactose, and an optional surfactant. The composition of the disclosure typically comprises from 0.1% to 100% w/w of the composition.

Another type of illustrative aerosol generator comprises a metered-dose inhaler. Metered-dose inhalers are pressurized aerosol dispensers, typically containing a suspension or solution composition of the tdsRNA in a liquefied propellant. During use these devices discharge the composition through a valve adapted to deliver a metered volume, typically from 10 μl to 200 μl, to produce a fine particle spray containing the tdsRNA. Suitable propellants include certain chlorofluorocarbon compounds, for example, dichlorodifluoromethane, trichlorofluoromethane, dichlorotetrafluoroethane and mixtures thereof. The composition may additionally contain one or more co-solvents, for example, ethanol, surfactants, such as oleic acid or sorbitan trioleate, antioxidant and suitable flavoring agents.

The preferred route and mode of administration will vary with the condition and age of the recipient, the nature of the infection or condition, and the chosen active ingredient.

### 7: Nasal Administration Devices

A device, encompassing a composition of the disclosure is also an embodiment.

The composition of the disclosure may be delivered by any nasal administration device or combination of devices. A combination refers to a composition that is both administered by two different devices or a device having the feature of two devices. Non-limiting examples of suitable devices that can be use individually or together include at least one selected from the group consisting of: a nebulizer; a sprayer (e.g., a spray bottle such as “Nasal Spray Pump w/Safety Clip, Pfeiffer SAP #60548; a squeeze bottle (e.g., bottle commonly used for nasal sprays, including ASTELIN (azelastine hydrochloride, Medpointe Healthcare Inc.) and PATANASE (olopatadine hydrochloride, Alcon, Inc.); a nasal pump spray (e.g., APTAR PHARMA nasal spray pump); a controlled particle dispersion devices (e.g., VIANASE electronic atomizer); a nasal aerosol device (e.g., ZETONNA nasal aerosol); a nasal nebulization device (e.g., EASYNOSE nebulizer, a pressure-driven jet nebulizer, or an ultrasonic nebulizer); a powder nasal delivery devices (e.g., OPTINOSE breath-powered nasal delivery device); an atomized nasal medication device (e.g., LMA MAD NASAL device); an instillation device; an inhalation device (e.g., an inhaler); a powder dispenser; a dry powder generator; an aerolizer (e.g., intrapulmonary aerosolizer or a sub-miniature aerosolizer, metered aerosol, powdered aerosol, spray aerosol); a spray; a metered spray; a metered dose inhalers (e.g., a propellant based metered-dose inhaler); a dry powder inhalation device; an intranasal instillation device; an intravesical instillation device; an insufflation device.

An application device for application to mucous membranes, such as, that of the nose, throat, and/or bronchial tubes (i.e., inhalation). This can be a swab, a pipette or a device for nasal irrigation, nasal rinse, or nasal lavage.

Another example is a syringe or plunger activated sprayer. This could be, for example, a sprayer head (or nozzle) attached, for example, via a Luer lock, to a syringe. The syringe applies a pressure to a composition that flows through the sprayer head and produces a spray or an aerosol.

More Specific Examples of Nasal Devices:

Aerosol: A product that is packaged under pressure and contains therapeutically active ingredients that are released upon activation of an appropriate valve system. For use as aerosols, the compounds of the present disclosure in solution or suspension may be packaged in a pressurized aerosol container together with suitable propellants, for example, hydrocarbon propellants like propane, butane, or isobutane with conventional adjuvants. The materials of the present disclosure also may be administered in aerosol but in a non-pressurized form such as in a nebulizer or atomizer.

Metered Aerosol: A pressurized dosage form comprised of metered-dose valves, which allow for the delivery of a uniform quantity of spray upon each activation.

Powdered Aerosol: A product that is packaged under pressure and contains therapeutically active ingredients in the form of a powder, which are released upon activation of an appropriate valve system.

Spray aerosol: An aerosol product that utilizes a compressed gas as the propellant to provide the force necessary to expel the product as a wet spray, it generally applicable to solutions of medicinal agents in aqueous solvents.

Spray: A liquid minutely divided as by a jet of air or steam. Nasal spray drug products contain therapeutically active ingredients dissolved or suspended in solutions or mixtures of excipients in non-pressurized dispensers.

Metered spray: A non-pressurized dosage form consisting of valves that allow the dispensing of a specified quantity of spray upon each activation.

Suspension spray: A liquid preparation containing solid particles dispersed in a liquid vehicle and in the form of course droplets or as finely divided solids.

Some non-limiting specific examples of commercially available devices are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

One illustrative type of solid particulate aerosol generator is an insufflator. Suitable compositions for administration by insufflation include finely comminuted powders which may be delivered by means of an insufflator or taken into the nasal cavity in the manner of a snuff. In the insulator, the powder (e.g., a metered-dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the composition.

A second type of illustrative aerosol generator comprises a metered-dose inhaler. Metered-dose inhalers are pressurized aerosol dispensers, typically containing a suspension or solution composition of the active ingredient in a liquefied propellant. During use these devices discharge the composition through a valve adapted to deliver a metered volume, typically from 10 to 200 ul, to produce a fine particle spray containing the active ingredient. Suitable propellants include certain chlorofluorocarbon compounds, for example, dichlorodifluoromethane, trichlorofluoromethane, dichlorotetrafluoroethane and mixtures thereof. The composition may additionally contain one or more co-solvents, for example, ethanol, surfactants, such as oleic acid or sorbitan trioleate, antioxidant and suitable flavoring agents.

It is noted that while some of the devices may dispense a liquid, the liquid may be a rapidly evaporating liquid which would turn into a dry powder before contact with a patient. Therefore, in effect, the spray can be considered a dry powder administration.

Any of the listed devices may be incorporated into an administration device embodiment of this disclosure.

### 8: Discussion of Further Embodiments and Features

Subject or Patient

As used herein, a “subject” has the same meaning as a “patient” and is a mammal, preferably, a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Other examples of subjects include any animal that may harbor coronaviruses such as civet cats, swine, cattle, horses, camels, cats, dogs, rodents, birds, bats, rabbits, ferrets, mink, snake, and the like. As used herein, the terms “patient” or “subject” are used interchangeably.

Analysis

Analysis of the base composition of RNA whether single-stranded or double-stranded may be performed by one of ordinary skill in the art. For example, the molar ratio of Cytidine to Uridine (C:U) in Poly C₁₂U can be determined by hydrolysis of the polymer to its constituent nucleotides. The nucleotides are separated by High Performance Liquid Chromatography and quantitated by ultraviolet absorption. The polymers were hydrolyzed by RNase. An example of a solvent system, which can be used for the gradient chromatography is 100 mM Triethylammonium acetate, pH 6.0 and Acetonitrile.

Devices and Kits

In another aspect, the present disclosure relates to and comprises a therapeutic device for intranasal delivery. In one embodiment, the therapeutic device may comprise any suitable devices charged with a preparation of tdsRNA and optionally, another biologically active agent such as a vaccine or antigen. These devices are described in more detail below.

Additional Methods and Compositions

In any aspect of this disclosure, the method may comprise a further step of administering to the subject one or more compound or agent selected from the group consisting of: antiviral, interferon, interferon mixture, Alferon, alpha-interferon species, recombinant or natural interferon-alpha, recombinant or natural interferon-alpha-2a, recombinant or natural interferon-beta, recombinant or natural interferon-beta-1b, and recombinant or natural interferon-gamma.

The alpha-interferon species may be a mixture of at least seven species of alpha-interferon produced by human white blood cells. The seven species may be, for example, interferon alpha 2, interferon alpha 4, interferon alpha 7, interferon alpha 8, interferon alpha 10, interferon alpha 16, and interferon alpha 17.

In another aspect, the agent may be one or more selected from the group consisting of Remdesivir, chloroquine, hydroxychloroquine, oseltamivir, zanamivir, abacavir, zidovudine, zalcitabine, didanosine, stavudine, efavirenz, indinavir, ritonavir, nelfinavir, amprenavir, ribavirin, interleukin, IL-2, PD-L1, Anti-PD-L1, checkpoint inhibitor, peramivir, and neuraminidase inhibitors.

The compositions and methods of this disclosure may comprise any compound/agent discussed herein including, e.g., in this previous paragraph.

Effective Amount: Therapeutically or Prophylactically Effective Amount

The compositions are delivered in effective amounts. The term “effective amount” refers to the amount necessary or sufficient to realize a desired biologic effect which is, for example, inhibiting, attenuating, preventing or at least reducing the risk of SARS-CoV-2 infection (SARS-CoV-2 infection). In addition to the sample dosages and administration methods mentions, one of ordinary skill in the art can empirically determine the effective amount of the tdsRNA without necessitating undue experimentation. It is preferred generally that a maximum dose be used, that is, the highest safe dose according to medical judgment.

Effective dosage forms, modes of administration, and dosage amounts may be determined empirically, and making such determinations is within the skill of the art. It is understood by those skilled in the art that the dosage amount will vary with the route and mode of administration, the rate of excretion, the duration of the treatment, the identity of any other drugs (e.g., antiviral agent) being co-administered, the age, size, species of mammal (e.g., human patient), and other factors well known in the arts of medicine and veterinary medicine. In general, a suitable dose of any active agent disclosed herein or a composition containing the same will be that amount of the active agent or composition, which is the lowest dose effective to produce the desired effect. The desired effect may be to reduce the severity or duration of a symptom of a viral infection. The desired effect may be to prevent infection of a subject even if the subject was challenged by exposure to a virus such as a SARS-CoV-2 virus.

### 9: Virus Vaccine

One aspect of the disclosure is directed to the making of virus vaccine such as SARS-CoV-2 vaccine, to vaccines made using the methods of the disclosure, and also to methods of using vaccines that are either made using the disclosed methods or made using another method.

Culturing SARS-CoV-2 and Coronaviruses

Methods for culturing coronaviruses and SARS-CoV-2 in particular are published and examples of these methods are disclosed in the Examples section of this disclosure.

Virus Inactivation

The term “inactivated” in the context of an inactivated SARS-CoV-2 virus means that the virus is incapable of replication in vivo or in vitro. For example, the term inactivated encompasses a virus that has been replicated (e.g., in vitro) and then killed using chemical or physical means such that it is no longer capable of replicating.

Methods of inactivating a virus and using the virus as a component of a vaccine are known. The United States Department of Agriculture has approved protocols for using binary ethylene-imine or formaldehyde to inactivate certain viruses for vaccine production. These methods are disclosed in numerous publications such as, for example, in U.S. Pat. Nos. 5,459,073; 5,811,099; 5,849,517; 5,811,099; 5,849,517; 7,252,984; 8,278,083 and published U.S. Patent Appl. 2011/0110975. These patents and patent applications are incorporated herein by reference.

As a non-limiting example, coronavirus may be inactivated as follows using binary ethylene-imine: Stock Solution A of 0.2 molar bromoethylamine hydrobromide is prepared by adding 40.98 grams to deionized water and made up to 1000 ml. Solution B is sodium hydroxide, 0.4 molar. This is prepared by adding 16 grams of NaOH to deionized water and made up to 1000 ml. The stock solutions are stored at room temperature until ready for use. Prior to usage equal volumes of Solution A and B are mixed and incubated at 37° C. to cyclize. The cyclized solution is then added at 2 percent vol/vol concentration to a liquid coronavirus preparation. The fluids are mixed thoroughly and incubated at 37° C. for 72 hours. At the end of this incubation, 20 ml of a sterile 1 molar sodium thiosulfate solution is added per Liter of virus to insure neutralization of the BEI. The pH of the preparation is further adjusted to neutral (e.g., pH=7.5) using, for example, NaOH after incubation.

As a further non-limiting example, influenza virus may be inactivated as follows: Formalin is added to the supernatant of an infected VERO cell culture (e.g., Vero E6) to a final formalin concentration of 0.025%). The virus is inactivated at 32° C. for 24 h.

If desired, the inactivated viruses described above may be further purified by one or more of the following techniques: (1) zonal centrifugation in a continuous sucrose gradient—for example, in a 0-50% sucrose gradient; (2) DNAse treatment or RNAse treatment; (3) diafiltration; and (4) and sterile filtration.

If desired, diluted and undiluted samples of the inactivated fluids are tested along with positive control and negative control. Positive control (positive for virus) may be virus solution treated with equivalent amount of media instead of inactivation chemicals like cyclized solution or sodium thiosulfate. Negative (negative for virus) control may be media that does not contain and that has not been exposed to coronavirus. The effectiveness of the inactivation may be confirmed by testing the titer of the inactivated virus preparation along with the positive and negative controls according to standard procedures. One procedure is described in the example section of this disclosure.

In additional to titering with in vitro infection and cell culture, the sequence of SARS-CoV-2 is known (see, Sequence Listing) and detection may be performed by PCR using probes that can be determined by one of ordinary skill in the art. As a further example, primer and probe sequences for polymerase chain reaction (PCR) of SARS-CoV-2 may be as follows:

Assay IA (Target: ORF1b-nsp14)
Forward primer (HKU-ORFlb-nsp14F):

(SEQ ID NO: 1)

5′-TGGGGYTTTACRGGTAACCT-3′
Reverse primer (HKU- ORF1b-nsp14R):

(SEQ ID NO: 2)

5′-AACRCGCTTAACAAAGCACTC-3′
Probe (HKU-ORFlb-nsp141P):

(SEQ ID NO: 3)

5′-FAM-TAGTTGTGATGCWATCATGACTAG-TAMRA-3′;
Assay IB (Target: N)
Forward primer (HKU-NF):

(SEQ ID NO: 4)

5′-TAATCAGACAAGGAACTGATTA-3′
Reverse primer (HKU-NR):

(SEQ ID NO: 5)

5′-CGAAGGTGTGACTTCCATG-3′
Probe (HKU-NP):

(SEQ ID NO: 6)

5′-FAM-GCAAATTGTGCAATTTGCGG-TAMRA-3′;
Assay IIA (Target: ORF1ab)
Forward primer:

(SEQ ID NO: 7)

5′-CCCTGTGGGTTTTACACTTAA-3′
Reverse primer:

(SEQ ID NO: 8)

5′-ACGATTGTGCATCAGCTGA-3′
Probe:

(SEQ ID NO: 9)

5′-FAM-CCGTCTGCGGTATGTGGAAAGGTTATGG-BHQ1-3′;
Assay IIB (Target: N)
Forward primer:

(SEQ ID NO: 10)

5′-GGGGAACTTCTCCTGCTAGAAT-3′
Reverse primer:

(SEQ ID NO: 11)

5′-CAGACATTTTGCTCTCAAGCTG-3′
Probe):

(SEQ ID NO: 12)

5′-FAM-TTGCTGCTGCTTGACAGATT-TAMRA-3′

Antigens for Vaccines

One embodiment of the disclosure relates to tdsRNA used alone.

Another embodiment of the disclosure relates to tdsRNA administered with a vaccine. A vaccine comprises one or more antigens that can trigger an immune response and produce immunity in a host subject.

The composition of the present disclosure can be used for immunization against SARS-CoV-2. Antigens for use against coronavirus including SARS-CoV-2 or other viruses in the present disclosure can be derived from a variety of virus strains. In the discussion of this section, the term “virus” includes any virus listed in this disclosure including various influenza virus, and coronavirus and including SARS-CoV-2.

Antigens that may be used in the present compositions, for example, in combination with tdsRNA, include, but are not limited to, proteins, peptides, peptidomimetics (synthetic peptides), carbohydrates (including monosaccharides, disaccharides, oligosaccharides and polysaccharides), lipids, nucleic acids (e.g., DNA and RNA), and conjugates/mixtures thereof. Nucleic acid antigens includes a DNA or RNA fragment, a DNA fragment incorporated into a vector such as a plasmid, ribozyme, antisense oligonucleotide, siRNA and small hairpin RNA. Examples of polynucleotide-containing antigens include, for example, (a) nucleic acid sequences that directly encode polypeptide-containing antigens (e.g., mRNA molecules) and (b) vector constructs that indirectly encode polypeptide-containing antigens, for example, vector constructs that express heterologous nucleic acid sequences, which in turn encode polypeptide-containing antigens (e.g., DNA vector constructs and RNA vector constructs). The antigen can be parts (e.g., coats, capsules, cell walls, flagella, fimbrae, and toxins) of microorganisms. The antigen can also be attenuated live microorganism or inactivated microorganisms. The microorganisms include, but are not limited to, viruses (e.g., influenza virus, avian influenza virus, parainfluenza virus, adenovirus, SARS virus, AIDS virus, cytomegalovirus, hepatitis virus, Japanese encephalitis virus, measles virus and the like and including all viruses listed in this disclosure), bacteria (e.g., Bacillus anthracis, Streptococcus pneumoniae, Neisseria meningitidis, Staphylococcus, Pseudomonas aeruginosa and the like), fungi (e.g., Cryptococcus, Aspergillus and the like), protozoan (e.g., malaria and the like), other microorganisms and toxin, cadaver of insect (e.g., mite and the like), pollen and the like. This includes also any pathogen and any viruses listed in this disclosure. The antigen usable for the composition of the present disclosure is not particularly limited as long as it affords an effective immune response. In certain embodiments, the immune response includes an increase in the intranasal IgA antibody titer and an increase in the blood IgG antibody titer, as well as to be protective against the antigen or the microorganism in the vaccinated subject.

Virus strains include killed, attenuated or inactivated virus as well as subunit antigens. The antigens include protein containing species, peptide-containing species, polysaccharide containing species. Also included are polynucleotide containing species that express an immunogenic protein or polypeptide. These polynucleotide species may be genetically engineered polynucleotide species such as DNA and RNA which can induce an immune response or induce the production of viral antigens in a host. U.S. Patent Application No. 2005/0063986 discusses recombinant DNA constructs containing wild type or mutant type viral antigens.

A number of alternative procaryotic (bacterial) expression systems have been developed for antigen production, including an Escherichia coli expression system (Vodkin et al. (1983) Cell 34:693-697), a Salmonella typhimurium expression system (Coulson et al. (1994) Vaccine 12:1395-1401), a Bacillus subtilis expression systems (see, e.g., U.S. Pat. No. 6,267,966 to Baillie; Ivins et al. (1986) Infection and Immunity 54:537-542; and Baillie et al. (1994) Let. Appl. Microbiol. 19:225-227). Moreover, the complete gene sequence for SARS-CoV-2 is known and publicly available, enabling the development and production of a wide variety of antigens, including polypeptide containing and polynucleotide containing antigens.

A peptide is a protein fragment comprising a short chain of amino acids, no less than two amino acids. A protein is generally a longer chain of amino acids, though there is no exact rule as to where a peptide ends and a protein begins. The general peptide/protein nomenclature also considers whether the structure is a whole molecule, such as insulin-like growth factor-1 (IGF-1) that is a 73 amino acids long peptide, or if the structure is a fragment of a protein molecule, such as a trypsin cleaved fragment of a protein that would normally be called a tryptic peptide.

In general, the peptides, peptidomimetics, and proteins used in nasal immunization have molecular weights on the order of about 150 to about 200,000 daltons, about 1,000 to about 180,000 daltons, about 2,000 to about 150,000 daltons, about 3,000 to about 100,000 daltons, about 50,000 to about 100,000 daltons, about 20,000 to about 50,000 daltons, or about 30,000 to about 50,000 daltons. In one embodiment the peptides used in the present disclosure have molecular weights on the order of about 150 to about 30,000 daltons, though other peptides, which, due to their tertiary or quaternary structure may be larger than 30,000 daltons, are also within the scope of the disclosure. In certain embodiments, the peptides used in the present disclosure have molecular weights on the order of about 150 to about 10,000 daltons, or about 150 to about 7,000 daltons.

Proteins and peptides may be generated by recombinant techniques. Thus, chimeric molecules containing regions from different proteins may be used. For example, a recombinant protein containing the Plasmodium falciparum malaria circumsporozoite repeat region fused to a section of the Hepatitis B core antigen may be used. Milich et al., Conversion of poorly immunogenic malaria repeat sequences into a highly immunogenic vaccine candidate, Vaccine, Volume 20, Issues 5-6, (2001) Pages 771-788. It is notes that the sequence of SARS-CoV-2 and many pathogens are known and published.

The compositions of the present disclosure may be used for immunization against one or more than one type of microorganism or allergen. The compositions may contain one type of antigen or more than one type of antigen.

The production of vaccines is exemplified below using a model virus—SARS-CoV-2. SARS-CoV-2 is a single-stranded RNA-enveloped virus which is 29,881 bp in length (GenBank no. MN908947), encoding 9860 amino acids. In this genome, the S, E, M, and N genes encode structural proteins while other open reading frame encode non structural proteins such as 3-chymotrypsin-like protease, papain-like protease, and RNA-dependent RNA polymerase.

The surface of the SARS-CoV-2 is covered by glycosylated S proteins that bind to the host cell receptor angiotensin-converting enzyme 2 (ACE2), mediating viral cell entry. The S proteins are thus potential targets for vaccine production. With a size of 180-200 kDa, the S protein consists of an extracellular N-terminus, a transmembrane (TM) domain anchored in the viral membrane, and a short intracellular C-terminal segment. The total length of SARS-CoV-2 S is 1273 aa and consists of a signal peptide (amino acids 1-13) located at the N-terminus, the 51 subunit (14-685 residues), and the S2 subunit (686-1273 residues); the last two regions are responsible for receptor binding and membrane fusion, respectively. In the native state, the CoV S protein exists as an inactive precursor. During viral infection, target cell proteases activate the S protein by cleaving it into S1 and S2 subunits, which is necessary for activating the membrane fusion domain after viral entry into target cells.

By analyzing the structure of the S1 subunit and S2 subunits, a number of subregions of these subunits are found to be good candidates (i.e., good antigens) for vaccine productions. These regions include, RBD situated in the S1 subunit, NTD and CTD, and the heptad repeat (HR). The whole S protein, each of the subunits, or the domains of the subunits may be used as antigens. The antigens may be purified or cloned and expressed. Further, RNA encoding each of these proteins, subunits and domains may be used to construct RNA vaccines. Each of the vaccines discussed may be combined with tdsRNA in a method or a composition of this disclosure.

The compositions of the present disclosure may be used for immunization against one or more than one type of microorganism or allergen. The compositions may contain one type of antigen or more than one type of antigen, or RNA or RNAs encoding the same.

The antigen is present in the composition in a therapeutically effective amount. In general the antigen is present in an amount of about 0.001 to about 50 wt. % of the composition, about 0.01 to about 30 wt. %, about 0.1 to about 20 wt. %, about 0.1 to about 10 wt. %, or about 0.1 to about 2 wt. % of the composition.

The antigen of the present disclosure may be used in a comparatively crude state, or may be purified before use. For purification, for example, a method conventionally used in the art for the purification of a peptide, protein, DNA, RNA, carbohydrate, may be carried out in the present disclosure, such as filtration, concentration, centrifugation, gel filtration chromatography, ion exchange chromatography, hydrophobic chromatography, adsorption chromatography, high performance liquid chromatography, affinity chromatography, gel electrophoresis, isoelectric focusing and the like. When necessary, these methods may be combined as appropriate. According to the form of final use, purified antigen may be concentrated or freeze-dried to give a liquid or solid.

The pharmaceutical compositions of the present disclosure may also be used in desensitization. For example, increasing doses of an allergen are administered to a subject who has demonstrated sensitivity to the allergen. Examples of allergen doses used for desensitization are known in the art, see, for example, Formadley (1998) Otolaryngol. Clin. North Am. 31:111-127.

At least one immunological adjuvant may be used in the present composition to assist or modify the action of an antigen. Immunological adjuvants may lead to one or more of the following effects, among others: an increased immune response, a more diversified immune response, an accelerated immune response, a more persistent/prolonged immune response. Adjuvants that may be used in the present disclosure include, but are not limited to, dextran or cyclodextran and saponin.

Non-limiting examples of adjuvants include: (1) aluminum salts (alum), such as aluminum hydroxide, aluminum phosphate, aluminum sulfate, etc.; (2) submicron emulsions comprising a metabolizable oil, such as squalene, and an emulsifying agent, such as one or more sorbitan derivatives, for example, (a) MF59 (International Publication No. WO90/14837; Chapter 10 in Vaccine design: the subunit an adjuvant approach, Eds. Powell & Newman, Plenum Press 1995), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribij adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (DetoxJ) (for a further discussion of suitable submicron oil-in-water emulsions for use herein, see commonly owned, patent application Ser. No. 09/015,736, filed on Jan. 29, 1998); (d) saponin adjuvants, such as Quil A, or QS21 (e.g., Stimulonj (Cambridge Bioscience, Worcester, Mass.)) may be used or particles generated therefrom such as ISCOMs (immunostimulating complexes), which ICOMS may be devoid of additional detergent e.g., WO00/07621; (e) Complete Freunds Adjuvant (CFA) and Incomplete Freunds Adjuvant (IFA); (f) cytokines, such as interleukins (e.g., IL-1, IL-2, IL-4, IL-5, IL-6, IL-7, IL-12 (WO99/44636), etc.), interferons (e.g., gamma interferon), macrophage colony-stimulating factor (M-CSF), tumor necrosis factor (TNF), etc.; (g) phospholipid adjuvants, including lipopolysaccharide and liposaccharide phosphate adjuvants, for example, monophosphoryl lipid A (MPL) and its derivatives, 3-O-deacylated MPL (3dMPL) e.g., GB-2220221, EP-A-0689454, optionally in the substantial absence of alum when used with pneumococcal saccharides e.g., WO00/56358; as well as aminoalkyl glucosamine phosphate compounds such as those described in U.S. Pat. No. 6,355,257; (h) combinations of 3dMPL with, for example, QS21 and/or oil-in-water emulsions, e.g., EP-A-0835318, EP-A-0735898, EP-A-0761231; (i) a polyoxyethylene ether or a polyoxyethylene ester e.g. WO99/52549; (j) a polyoxyethylene sorbitan ester surfactant in combination with an octoxynol (WO01/21207) or a polyoxyethylene alkyl ether or ester surfactant in combination with at least one additional non-ionic surfactant such as an octoxynol (WO01/21152); (k) a saponin and an immunostimulatory oligonucleotide (e.g., a CpG oligonucleotide) (WO00/62800); (1) an immunostimulant and a particle of metal salt e.g. WO00/23105; (m) a saponin and an oil-in-water emulsion e.g. WO99/11241; (n) a saponin (e.g. QS21)+3dMPL+IL-12 (optionally+a sterol) e.g. WO98/57659; (o) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CT-S109 (where serine is substituted for the wild-type amino acid at position 109), and PT-K9/G129 (where lysine is substituted for the wild-type amino acid at position 9 and glycine substituted at position 129) (see, e.g., International Publication Nos. WO93/13202 and WO92/19265); (p) aminoalkyl glucosaminide 4-phosphates (AGP's), see, e.g., Johnson, D. A. et al.; Bioorg. Med. Chem. Lett., 1999 Aug. 2; 9(15):2273-8, (q) imidazoquinolines such as imiquimod (R-837) and resiquimod (R-848), see, e.g., Vasilakos, J. P. et al.; Cell. Immunol. 2000 Aug. 25; 204(1):64-74, (r) lipopolysaccharide mimetics (including monophosphoryl lipid A mimetics), such as non-saccharide phospholipids (e.g., simplified lipid A analogs lacking a disaccharide) described in Hawkins, L. D. et al; J. Pharmacol. Exp. Ther., 2002 February; 300(2):655-61 and U.S. Pat. No. 6,290,973; and (s) other substances that act as immunostimulating agents to enhance the effectiveness of the composition. Muramyl peptides include, but are not limited to, N-acetyl-muramyl-L-threonyl-D-isoglutamine (thr-MDP), N-acteyl-normuramyl-L-alanyl-D-isogluatme (nor-MDP), N-acetylmuramyl-L-alanyl-D-isogluatminyl-L-alanine-2-(1′-2′-dipalmitoyl-s-n-glycero-3-huydroxyphosphoryloxy)-ethylamine (MTP-PE), etc. U.S. Patent Publication Nos. 20080317784, 20090214596 and 20100092526.

For additional examples of immunological adjuvants, see Vaccine Design, The Subunit and the Adjuvant Approach, Powell, M. F. and Newman, M. J, eds., Plenum Press, 1995.

Carrier and Additional Components

By way of illustration, the inactivated virus (e.g., SARS-CoV-2) may be mixed with a suitable carrier (e.g., water or saline) that optionally is buffered (e.g., phosphate buffered saline, such as Dulbecco's phosphate buffered saline “D-PBS”) before administering into a subject animal as a vaccine. Preferably, the carrier is such that the inactivated virus is uniformly dispersed in the resulting composition at the time of the administration, and it will not degrade the antigen-treated virus throughout a storage life of at least 10 days, more preferably at least one month at a temperature of about 0° C. to about 37° C. An example of one suitable solution includes a mixture of CaCl₂); MgCl₂; KCl; KH₂PO₄; NaCl; Na₂HPO₄; and D-Glucose (dextrose). More specifically, one example of such a solution is CaCl₂) at 0.901 mM; MgCl₂ at 0.493 mM; KCl at 2.67 mM; KH₂PO₄ at 1.47 mM; NaCl at 137.93 mM; Na₂HPO₄ at 8.06 mM; and D-Glucose (dextrose) at 5.56 mM.

A carrier or diluent for the vaccine may include one or any combination of stabilizers, preservatives and buffers. Suitable stabilizers may include, for example, SPGA, carbohydrates (such as sorbitol, mannitol, starch, sucrose, peptone, arginine, dextran, glutamate or glucose), proteins (such as dried milk serum, albumin or casein) or degradation products thereof. Suitable buffers may include for example alkali metal phosphates. Suitable preservatives may include thimerosal, merthuilate and gentamicin. Diluents include water, aqueous buffer (such as buffered saline) and polyols (such as glycerol). It will be appreciated that vaccine compositions herein, as well as any of its carrier or diluents are preferably free of any antibiotic, and/or any mercury-containing ingredient.

The vaccine may further comprise an adjuvant or additional reagent, such as an adjuvant selected from one or any combination of lecithin, a pharmaceutically acceptable polymer, saponin or a derivative thereof, or cholesterol. One preferred adjuvant or additional reagent is tdsRNA.

Administration of Inactivated Coronavirus

This disclosure further contemplates immunizing a subject with a vaccine composition described according to the present teachings. Thus the methods described herein may further comprise at least one step of administering a subject in need of immunization with the vaccine composition described in the present teachings. Any step of administering a subject in need of immunization with the vaccine composition by performed by ingestion (e.g., from drinking water), intranasally (e.g., by aerosol), intraocularly (e.g., by aerosol), via intramuscular injection, by subcutaneous injection, or any combination thereof.

A plurality of steps of administering the vaccine to the subject may be performed at intervals (e.g., they may be administered to the same subject on multiple occasions, such as at intervals of at least 3 days). By way of further example, at least two steps of administering the subject may performed at intervals of greater than one week (e.g., about 14 days apart). It is also possible that only a single dose is administered and is sufficient for achieving the desired satisfactory immune response.

The dosage of the vaccine preferably will be a sufficient amount for inducing immunity in the vaccinated subjects against challenge by a virulent form of the virus. Immunity can be described as the realization of an increased survival rate after a period of an immunized subject challenged by a virulent form of the virus. The period may be 4 days, 7 days, 14 days, or 28 days for example. Challenges include the step of exposing the virulent form of the virus to a subject in a 100% lethal dosage amount. Increased survival may be, for example, over 50%, over 75%, over 90%, over 95%, over 98%, or 100% survival rate in subjects exposed to a 100% lethal dose. Exposing in this context may be accidental exposure, occupational exposure, exposure during the course of regular disease transmission or a laboratory administration with non-human subjects.

Methods for determining the optimal dose of vaccine is well known. The dosage of the inactivated virus can be determined readily by the skilled artisan, for example, by first identifying doses effective to elicit a prophylactic or therapeutic immune response, e.g., by measuring the serum titer of virus-specific immunoglobulins or by measuring the inhibitory ratio of antibodies in serum samples, or urine samples, or mucosal secretions. Said dosages can be determined from animal studies. A non-limiting list of animals used to study the coronavirus and influenza virus include the guinea pig, Syrian hamster, chinchilla, hedgehog, chicken, rat, mouse and ferret. Some animals are not natural hosts to coronavirus or influenza viruses but can still serve in studies of various aspects of the disease. For example, any of the above animals can be dosed with a vaccine to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

Optionally, a unit dosage of inactivated virus (e.g., a coronavirus. e.g., SARS-CoV-2) or virus antigen may be as follows. For example, a dosage may be about 1 μg, about 5 μg, about 10 μg, about 20 μg, about 25 μg, about 30 μg, about 50 μg, about 100 μg, about 125 μg, about 150 μg, or about 200 μg. Alternatively, a dosage is less than about 1 μg, (for example about 0.08 μg, about 0.04 μg; about 0.2 μg, about 0.4 μg, about 0.8 μg, about 0.5 μg or less, about 0.25 μg or less, or about 0.1 μg or less), or more than about 125 μg, (for example about 150 μg or more, about 250 μg or more, or about 500 μg or more).

The dosages of (1) tdsRNA and (2) coronavirus antigen (or inactivated coronavirus) are disclosed and where a composition or method or mixture comprising both are made the dosage of each can be used for the combination.

Although this disclosure is illustrated in connection with immunization for prevention of SARS-CoV-2 Virus, the present disclosure is not intended to be so limited. It may have applications for immunization or for prevention of infection of other viruses, including but not limited to other viruses such as H5 influenza; H7 influenza; H5N1 influenza; West Niles Virus, Zika Virus, MERS virus; SARS virus and other influenza virus or coronavirus (examples of which are described in this disclosure). The compositions (e.g., vaccine) and methods of this disclosure may comprise a plurality of antigenic components suitable for immunizing against a plurality of viruses. For example, this disclosure contemplates methods and vaccines comprising antigens or viruses from both influenza virus and coronavirus.

Other Aspects Applicable to General Nasal Administration and to Nasal Vaccine

The nasal vaccination methods are not particularly limited as long as it can induce an immune response, for example, an immune response in the topical mucous membrane of the respiratory tract (particularly upper respiratory tract), which is an infection route of many immunogens such as bacterium and virus. Examples of the method include spraying, swabbing, dropwise addition and the like. The pharmaceutical composition can be administered intranasally by devices including, but not limited to, an intranasal spray device, an atomizer, a nebulizer, a metered dose inhaler (MDI), a pressurized dose inhaler, an insufflator, an intranasal inhaler, a nasal spray bottle, an unit dose container, a pump, a dropper, a squeeze bottle, or a bi-directional device. The pharmaceutical composition may be administered intranasally in the form of a gel, an ointment, a nasal emulsion, a lotion, a cream, a nasal tampon, or a bioadhesive strip. The nasal delivery device can be metered to administer an accurate effective dosage amount to the nasal cavity. The nasal delivery device can be for single unit delivery or multiple unit delivery. The compounds of the present disclosure may also be delivered through a tube, a catheter, a syringe, a packtail, a pledget, a nasal tampon or by submucosal infusion. See, e.g., U.S. Patent Publication Nos. 20090326275, 20090291894, 20090281522 and 20090317377.

In one embodiment, the composition of the present disclosure is delivered through a nasal spray applicator. If intra-nasal application is desired, the composition may be placed in an intra-nasal spray-dosing device or atomizer and may be applied by spraying it into the nostrils of a subject for delivery to the mucous membrane of the nostrils. A sufficient amount is applied to achieve the desired systemic or localized antigen levels. For an intra-nasal spray, up to about 200 microliters is typically applied, with an application of about 50 to about 150 microliters being preferred, and 75 to 120 microliters most preferred. One or more nostrils may be dosed and application may occur as often as desired or as often as is necessary. In preferred embodiments, the nasal spray applicator is selected to provide droplets of the composition of a mean size of from about 10 microns to about 200 microns. More generally the droplet size is from about 30 microns to about 100 microns.

The present disclosure provides a pharmaceutical composition for nasal immunization which is capable of inducing an effective immune response. In certain circumstances, an effective immune response may be protective. An immune response may be measured in vitro, in vivo and/or ex vivo. Examples of measurable immune responses include, but are not limited to, antigen-specific antibody production (including measuring specific antibody subclasses), secretion of cytokines (including, but not limited to, IL-1, IL-2, IL-4, IL-5, IL-6, IL-10, IL-12, IFN-alpha, IFN-beta, IFN-gamma, TNF-alpha), release of histamine, activation or expansion of lymphocyte populations such as NK cells, T lymphocytes, B lymphocytes, macrophages and the like. Methods for measuring specific antibody responses include enzyme-linked immunosorbent assay (ELISA) and are well known in the art. Measurement of numbers of specific types of lymphocytes such as CD⁴⁺ T cells can be achieved, for example, with fluorescence-activated cell sorting (FACS). Cytotoxicity assays can be performed for instance as described in Raz et al. (1994) Proc. Natl. Acad. Sci. USA 91:9519-9523. Serum concentrations of cytokines can be measured, for example, by ELISA. These and other assays to evaluate the immune response to an immunogen are well known in the art. See, for example, Selected Methods in Cellular Immunology (1980) Mishell and Shiugi, eds., W.H. Freeman and Co. U.S. Pat. No. 7,628,990.

A therapeutically effective amount of the tdsRNA by itself or with antigen may vary according to factors such as the kind of a tdsRNA, antigen, desired action, physical and medical conditions of the subject, such as age, body weight, etc. A therapeutically effective amount of a tdsRNA or an antigen can be determined by one of ordinary skill in the art without undue experimentation. Based on known immunization dosing regimen and the teachings herein, one skilled in the art can select the dosing regimen and dosage for a particular subject or subjects. The ability of antigen to induce an effective immune response or of an antibody to inhibit a measurable parameter can be evaluated in an animal model system predictive of efficacy in humans. For example, the ability of an SARS-CoV-2 antigen to protect animals such as ferrets from challenge with SARS-CoV-2 can predict efficacy in humans. Alternatively, this property of an antigen or antibody can be evaluated by examining the ability of the composition to modulate antigen/cell interactions, e.g., binding, infection, virulence, and the like, by in vitro assays known to the skilled practitioner. In vitro assays include binding assays, such as ELISA, and neutralization assays.

### 10: Viruses that can be Treated by any Embodiment of the Disclosure

tdsRNA, alone or in combination with other active ingredients, can be used to prevent and/or treat infection by one or more viruses. The other active ingredients may be vaccines. Vaccines may comprise viral antigens, whole viruses (e.g., a less virulent strain), attenuated viruses, inactivated viruses, and the like. The viruses that can be treated by tdsRNA and the methods of this disclosure may be one as listed herein.

Influenza

Influenza is widely regarded as the most dangerous virus threat. Its various strains are constantly changing, sometimes through subtle mutations and sometimes through more drastic genomic changes. Even in nonpandemic years, influenza kills up to 500,000 people around the globe. Influenza viruses belong to the Orthomyxoviridae family of viruses, which includes five genera: Influenzavirus A, Influenzavirus B, Influenzavirus C, Isavirus, and Thogotovirus. Dhori virus is a species of the genus Thogotovirus. An influenza virus can infect humans and other species. Influenza type A viruses can infect humans, birds, pigs, horses, seals and other animals. Wild birds can be natural hosts for these viruses.

Influenza Type A

Influenza type A viruses can be divided into subtypes and named on the basis of two proteins on the surface of the virus: hemagglutinin (HA) and neuraminidase (NA). For example, an “H7N2 virus” designates an influenza A subtype that has an HA7 protein and an NA2 protein. Similarly an “H5N1” virus has an HA 5 protein and an NA 1 protein. There are 16 known HA subtypes and 9 known NA subtypes. Many different combinations of HA and NA proteins are possible. Any number of the known HA subtypes (HAL HA2, HA3, HA4, HAS, HA6, HA7, HA8, HA9, HA10, HA11, HA12, HA13, HA14, HA15, and HA16) can be combined with any number of the known NA subtypes (NA1, NA2, NA3, NA4, NAS, NA6, NA7, NAB, and NA9) to produce a vaccine to prevent or treat an infection. The HA and NA subtypes can also be used individually in a vaccine to prevent infection. Different subtype vaccines can be combined at the point of use, either sequentially or simultaneously, to prevent an infection. Some influenza A subtypes (e.g., H1N1, H1N2, and H3N2) are currently in general circulation among people. Other subtypes can be found in other animal species. For example, H7N7 and H3N8 viruses can cause illness in horses, and H3N8 also has recently been shown to cause illness in dogs.

Influenza Type B

Influenza B viruses can be found in humans and can also infect seals. Unlike influenza A viruses, these viruses are not classified according to subtype. Influenza B viruses can cause morbidity and mortality among humans, but in general are associated with less severe epidemics than influenza A viruses. Although influenza type B viruses can cause human epidemics, they have not caused pandemics.

Influenza Type C

Influenza type C viruses can cause mild illness in humans and do not cause epidemics or pandemics. These viruses can also infect dogs and pigs. These viruses are not classified according to subtype.

Coronavirus Including SARS-CoV-2

Coronavirus has emerged as a new threat to human health and safety. Prior to 2002, coronaviruses were not considered to be significant human pathogens. Known coronaviruses such as HCoV-229E and HCoV-OC43 result in only mild respiratory infections in healthy adults. In 2002, however, severe acute respiratory syndrome coronavirus (SARS-CoV) emerged in Guangdong Province, China. This virus rapidly spread to 29 different countries, resulting in 8,273 confirmed cases and 775 (9%) deaths (1). In 2012, Middle East respiratory syndrome coronavirus (MERS-CoV) was detected in a patient with severe respiratory disease in Saudi Arabia. According to the World Health Organization, 2494 laboratory-confirmed cases have been reported with a total of 858 deaths (37.1%). In 2019 and 2020, SARS-CoV-2 (also called COVID-19, “2019-nCoV” or “Wuhan coronavirus” and other names before it was standardized as “SARS-CoV-2”) emerged in Wuhan China and by early March 2020 there have been 80859 infections causing 3100 deaths (3.8%). The high case fatality rate, vaguely defined epidemiology, and absence of prophylactic or therapeutic measures against these coronaviruses have created an urgent need for an effective vaccine and related therapeutic agents.

Other Viruses that May be Treated

The methods and compositions described herein can be useful for the prevention and/or treatment of infection by any virus, including, for example, at least the viruses listed below. An embodiment of the disclosure is the nasal administration of tdsRNA alone, or tdsRNA with other antiviral agents, for the treatment or prevention of an infection by any of the viruses below—including any combination of viruses. Examples of additional viruses that can be treated is listed in Table 2 below.

TABLE 2

Abelson leukemia virus,
Abelson murine leukemia virus,
Abelson's virus,
Acute laryngotracheobronchitis virus,
Adelaide River virus,
Adeno associated virus group,
Adenovirus,
African horse sickness virus,
African swine fever virus,
AIDS virus,
Aleutian mink disease parvovirus,
Alpharetrovirus,
Alphavirus,
ALV related virus,
Amapari virus,
Aphthovirus,
Aquareovirus,
Arbovirus,
Arbovirus C,
arbovirus group A,
arbovirus group B,
Arenavirus group,
Argentine hemorrhagic fever virus,
Argentine hemorrhagic fever virus,
Arterivirus,
Astrovirus,
Ateline herpesvirus group,
Aujezky's disease virus,
Aura virus,
Ausduk disease virus,
Australian bat lyssavirus,
Aviadenovirus,
avian erythroblastosis virus,
avian infectious bronchitis virus,
avian leukemia virus,
avian leukosis vims,
avian lymphomatosis virus,
avian myeloblastosis vims,
avian paramyxovirus,
avian pneumoencephalitis virus,
avian reticuloendotheliosis virus,
avian sarcoma virus,
avian type C retrovirus group,
Avihepadnavirus,
Avipoxvirus,
B virus,
B19 virus,
Babanki virus,
baboon herpesvirus,
baculovirus.
Barmah Forest virus,
Bebaru virus,
Berrimah virus,
Betaretrovirus,
Birnavirus,
Bittner virus,
BK virus,
Black Creek Canal virus,
bluetongue virus,
Bolivian hemorrhagic fever virus
Boma disease virus,
border disease of sheep virus,
borna virus,
bovine alphaherpesvirus 1,
bovine alphaherpesvirus 2,
bovine coronavirus,
bovine ephemeral fever virus,
bovine immunodeficiency virus,
bovine leukemia virus,
bovine leukosis virus,
bovine mammillitis virus,
bovine papillomavirus,
bovine papular stomatitis virus,
bovine parvovirus,
bovine syncytial virus,
bovine type C oncovirus,
bovine viral diarrhea virus,
Buggy Creek virus,
bullet shaped virus group,
Bunyamwera virus supergroup,
Bunyavirus,
Burkitt's lymphoma virus,
Bwamba Fever,
CA virus,
Calicivirus,
California encephalitis virus,
camelpox virus,
canarypox virus,
canid herpesvirus,
canine coronavirus,
canine distemper virus,
canine herpesvirus,
canine minute virus,
canine parvovirus,
Cano Delgadito virus,
caprine arthritis virus,
caprine encephalitis virus,
Caprine Herpes Virus,
Capripox virus,
Cardio virus,
caviid herpesvirus 1,
Cercopithecid herpesvirus 1,
cercopithecine herpesvirus 1,
Cercopithecine herpesvirus 2,
Chandipura virus,
Changuinola virus,
channel catfish virus,
Charleville virus,
chickenpox virus,
Chikungunya virus,
chimpanzee herpesvirus,
chub reovirus,
churn salmon virus,
Cocal virus,
Coho salmon reovirus,
coital exanthema virus,
Colorado tick fever virus,
Coltivirus,
Columbia SK virus,
common cold virus,
contagious eethyma virus,
contagious pustular dermatitis virus,
Coronavirus including 229E (alpha);
NL63 (alpha); OC43 (beta); HKU1 (beta);
MERS-CoV; MERS; SARS-CoV;
SARS-CoV-2; variants; strains, and
mutants thereof;
Corriparta virus,
coryza virus,
cowpox virus,
coxsackie virus,
CPV (cytoplasmic polyhedrosis virus),
cricket paralysis virus,
Crimean-Congo hemorrhagic fever virus,
croup associated virus,
Cryptovirus,
Cypovirus,
Cytomegalovirus,
cytomegalovirus group,
cytoplasmic polyhedrosis virus,
deer papillomavirus,
deltaretrovirus,
dengue virus,
Densovirus,
Dependovirus,
Dhori virus,
diploma virus,
Drosophila C virus,
duck hepatitis B virus,
duck hepatitis virus 1,
duck hepatitis virus 2,
duovirus,
Duvenhage virus,
Deformed wing virus DWV,
eastern equine encephalitis virus,
eastern equine encephalomyelitis virus,
EB virus,
Ebola virus,
Ebola-like virus,
echo virus,
echovirus,
echovirus 10,
echo virus 28,
echo virus 9,
ectromelia virus,
EEE virus,
EIA virus,
EIA virus,
encephalitis virus,
encephalomyocarditis group virus,
encephalomyocarditis virus,
Enterovirus,
enzyme elevating virus,
enzyme elevating virus (LDH),
epidemic hemorrhagic fever virus,
epizootic hemorrhagic disease virus
Epstein-Barr virus,
equid alphaherpesvirus 1,
equid alphaherpesvirus 4,
equid herpesvirus 2,
equine abortion virus,
equine arteritis virus,
equine encephalosis virus,
equine infectious anemia virus,
equine morbillivirus,
equine rhinopneumonitis virus,
equine rhinovirus,
Eubenangu virus,
European elk papillomavirus,
European swine fever virus,
Everglades virus,
Eyach virus,
felid herpesvirus 1,
feline calicivirus,
feline fibrosarcoma virus,
feline herpesvirus,
feline immunodeficiency virus,
feline infectious peritonitis virus,
feline leukemia/sarcoma virus,
feline leukemia virus,
feline panleukopenia virus,
feline parvovirus,
feline sarcoma virus,
feline syncytial virus,
Filovirus,
Flanders virus,
Flavivirus,
foot and mouth disease virus,
Fort Morgan virus,
Four Corners hantavirus,
fowl adenovirus 1,
fowlpox virus,
Friend virus,
Gammaretrovirus,
GB hepatitis virus,
GB virus,
German measles virus,
Getah virus,
gibbon ape leukemia virus,
glandular fever virus,
goatpox virus,
golden shinner virus,
Gonometa virus,
goose parvovirus,
granulosis virus,
Gross' virus,
ground squirrel hepatitis B virus,
group A arbovirus,
Guanarito virus,
guinea pig cytomegalovirus,
guinea pig type C virus,
Hantaan virus,
Hantavirus,
hard clam reovirus,
hare fibroma virus,
HCMV (human cytomegalovirus),
hemadsorption virus 2,
hemagglutinating virus of Japan,
hemorrhagic fever virus,
hendra virus,
Henipaviruses,
Hepadnavirus,
hepatitis A virus,
hepatitis B virus group,
hepatitis C virus,
hepatitis D virus,
hepatitis delta virus,
hepatitis E virus,
hepatitis F virus,
hepatitis G virus,
hepatitis nonA nonB virus,
hepatitis virus,
hepatitis virus (nonhuman),
hepatoencephalomyelitis reovirus 3,
Hepatovirus,
heron hepatitis B virus,
herpes B virus,
herpes simplex virus,
herpes simplex virus 1,
herpes simplex virus 2,
herpesvirus,
herpesvirus 7,
Herpesvirus ateles,
Herpesvirus hominis,
Herpesvirus infection,
Herpesvirus saimiri,
Herpesvirus suis,
Herpesvirus varicellae.
Highlands J virus,
Hirame rhabdovirus,
hog cholera virus,
human adenovirus 2,
human alphaherpesvirus 1,
human alphaherpesvirus 2,
human alphaherpesvirus 3,
human B lymphotropic virus,
human betaherpesvirus 5,
human coronavirus,
human cytomegalovirus group,
human foamy virus,
human gammaherpesvirus 4,
human gammaherpesvirus 6,
human hepatitis A virus,
human herpesvirus 1 group,
human herpesvirus 2 group,
human herpesvirus 3 group,
human herpesvirus 4 group,
human herpesvirus 6,
human herpesvirus 8,
human immunodeficiency virus,
human immunodeficiency virus 1,
human immunodeficiency virus 2,
human papillomavirus,
human T cell leukemia virus,
human T cell leukemia virus I,
human T cell leukemia virus II,
human T cell leukemia virus III,
human T cell lymphoma virus I,
human T cell lymphoma virus II,
human T cell lymphotropic virus type 1,
human T cell lymphotropic virus type 2,
human T lymphotropic virus I,
human T lymphotropic virus II,
human T lymphotropic virus III,
Ichnovirus,
infantile gastroenteritis virus,
infectious bovine rhinotracheitis virus,
infectious haematopoietic necrosis virus,
infectious pancreatic necrosis virus,
influenza virus A,
influenza virus B,
influenza virus C,
influenza virus D,
influenza virus pr8,
insect iridescent virus,
insect virus,
iridovirus,
Japanese B virus,
Japanese encephalitis virus,
JC virus,
Junin virus,
Kaposi's sarcoma-associated herpesvirus,
Kemerovo virus,
Kilham's rat virus,
Klamath virus,
Kolongo virus,
Korean hemorrhagic fever virus,
kumba virus,
Kysanur forest disease virus,
Kyzylagach virus,
La Crosse virus,
lactic dehydrogenase elevating virus,
lactic dehydrogenase virus,
Lagos bat virus,
Langur virus,
lapine parvovirus,
Lassa fever virus,
Lassa virus,
latent rat virus,
LCM virus,
Leaky virus,
Lentivirus,
Leporipoxvirus,
leukemia virus,
leukovirus,
lumpy skin disease virus,
lymphadenopathy associated virus,
Lymphocryptovirus,
lymphocytic choriomeningitis virus,
lymphoproliferative virus group,
Machupo virus,
mad itch virus,
mammalian type B oncovirus group,
mammalian type B retroviruses,
mammalian type C retrovirus group,
mammalian type D retroviruses,
mammary tumor virus,
Mapuera virus,
Marburg virus,
Marburg-like virus,
Mason Pfizer monkey virus,
Mastadenovirus,
Mayaro virus,
ME virus,
measles virus,
Menangle virus,
Mengo virus,
Mengovirus,
Middelburg virus,
milkers nodule virus,
mink enteritis virus,
minute virus of mice,
MLV related virus,
MM virus,
Mokola virus,
Molluscipoxvirus,
Molluscum contagiosum virus,
monkey B virus,
monkeypox virus,
Mononegavirales,
Morbillivirus,
Mount Elgon bat virus,
mouse cytomegalovirus,
mouse encephalomyelitis virus,
mouse hepatitis virus,
mouse K virus,
mouse leukemia virus,
mouse mammary tumor virus,
mouse minute virus,
mouse pneumonia virus,
mouse poliomyelitis virus,
mouse polyomavirus,
mouse sarcoma virus,
mousepox virus,
Mozambique virus,
Mucambo virus,
mucosal disease virus,
mumps virus,
murid betaherpesvirus 1,
murid cytomegalovirus 2,
murine cytomegalovirus group,
murine encephalomyelitis virus,
murine hepatitis virus,
murine leukemia virus,
murine nodule inducing virus,
murine polyomavirus,
murine sarcoma virus,
Muromegalovirus,
Murray Valley encephalitis virus,
myxoma virus,
Myxovirus,
Myxovirus multiforme,
Myxovirus parotitidis,
Nairobi sheep disease virus,
Nairovirus,
Nanirnavirus,
Nariva virus,
Ndumo virus,
Neethling virus,
Nelson Bay virus,
neurotropic virus,
New World Arenavirus,
newborn pneumonitis virus,
Newcastle disease virus,
Nipah virus,
noncytopathogenic virus,
Norwalk virus,
nuclear polyhedrosis virus (NPV),
nipple neck virus,
O'nyong'nyong virus,
Ockelbo virus,
oncogenic virus,
oncogenic viruslike particle,
oncornavirus,
Orbivirus,
Orf virus,
Oropouche virus,
Orthohepadnavirus,
Orthomyxovirus,
Orthopoxvirus,
Orthoreovirus,
Orungo,
ovine papillomavirus,
ovine catarrhal fever virus,
owl monkey herpesvirus,
Palyam virus,
Papillomavirus,
Papillomavirus sylvilagi,
Papovavirus,
parainfluenza virus,
parainfluenza virus type 1,
parainfluenza virus type 2,
parainfluenza virus type 3,
parainfluenza virus type 4,
Paramyxovirus,
Parapoxvirus,
paravaccinia virus,
Parvovirus,
Parvovirus B 19,
parvovirus group,
Pestivirus,
Phlebovirus,
phocine distemper virus,
Picodnavirus,
Picornavirus,
pig cytomegalovirus-pigeonpox virus,
Piry virus,
Pixuna virus,
pneumonia virus of mice,
Pneumovirus,
poliomyelitis virus,
poliovirus,
Polydnavirus,
polyhedral virus,
polyoma virus,
Polyomavirus,
Polyomavirus bovis,
Polyomavirus cercopi theci,
Polyomavirus hominis 2,
Polyomavirus maccacae 1,
Polyomavirus muris 1,
Polyomavirus muris 2,
Polyomavirus papionis 1,
Polyomavirus papionis 2,
Polyomavirus sylvilagi,
Pongine herpesvirus 1,
porcine epidemic diarrhea virus,
porcine hemagglutinating encephalomyelitis virus,
porcine parvovirus,
porcine transmissible gastroenteritis virus,
porcine type C virus,
pox virus,
poxvirus,
poxvirus variolae.
Prospect Hill virus,
Provirus,
pseudocowpox virus,
pseudorabies virus,
psittacinepox virus,
quailpox virus,
rabbit fibroma virus,
rabbit kidney vaculolating virus,
rabbit papillomavirus,
rabies virus,
raccoon parvovirus,
raccoonpox virus,
Ranikhet virus,
rat cytomegalovirus,
rat parvovirus,
rat virus,
Rauscher's virus,
recombinant vaccinia virus,
recombinant virus,
reovirus,
reovirus 1,
reovirus 2,
reovirus 3,
reptilian type C virus,
respiratory infection virus,
respiratory syncytial virus,
respiratory virus,
reticuloendotheliosis virus,
Rhabdovirus,
Rhabdovirus carpia,
Rhadinovirus,
Rhinovirus,
Rhizidiovirus,
Rift Valley fever virus,
Riley's virus,
rinderpest virus,
RNA tumor virus,
Ross River virus,
Rotavirus,
rougeole virus,
Rous sarcoma virus,
rubella virus,
rubeola virus,
Rubivirus,
Russian autumn encephalitis virus,
SA 11 simian virus,
SA2 virus,
Sabia virus,
Sagiyama virus,
Saimirine herpesvirus 1,
salivary gland virus,
sandfly fever virus group,
Sandjimba virus,
SARS virus,
SDAV (sialodacryoadenitis virus),
sealpox virus,
Semliki Forest Virus,
Seoul virus,
sheeppox virus,
Shope fibroma virus,
Shope papilloma virus,
simian foamy virus,
simian hepatitis A virus,
simian human immunodeficiency virus
simian immunodeficiency virus,
simian parainfluenza virus,
simian T cell lymphotrophic virus,
simian virus,
simian virus 40,
Simplexvirus,
Sin Nombre virus,
Sindbis virus,
smallpox virus,
South American hemorrhagic fever viruses,
sparrowpox virus,
Spumavirus,
squirrel fibroma virus,
squirrel monkey retrovirus,
SSV 1 virus group,
STLV (simian T lymphotropic virus) type I,
STLV (simian T lymphotropic virus) type II,
STLV (simian T lymphotropic virus) type III,
stomatitis papulosa virus,
submaxillary virus,
suid alphaherpesvirus 1,
suid herpesvirus 2,
Suipoxvirus,
swamp fever virus,
swinepox virus,
Swiss mouse leukemia virus,
TAC virus,
Tacaribe complex virus,
Tacaribe virus,
Tanapox virus,
Taterapox virus,
Tench reovirus,
Theiler's encephalomyelitis virus,
Theiler's virus,
Thogoto virus,
Thottapalayam virus,
Tick borne encephalitis virus,
Tioman virus,
Togavirus,
Torovirus,
tumor virus,
Tupaia virus,
turkey rhinotracheitis virus,
turkeypox virus,
type C retroviruses,
type D oncovirus,
type D retrovirus group,
ulcerative disease rhabdovirus,
Una virus,
Uukuniemi virus group,
vaccinia virus,
vacuolating virus,
varicella zoster virus,
Varicellovirus,
Varicola virus,
variola major virus,
variola virus,
Vasin Gishu disease virus,
VEE virus,
Venezuelan equine encephalitis virus,
Venezuelan equine encephalomyelitis virus,
Venezuelan hemorrhagic fever virus,
vesicular stomatitis virus,
Vesiculovirus,
Vilyuisk virus,
viper retrovirus,
viral haemorrhagic septicemia virus,
Visna Maedi virus,
Visna virus,
volepox virus,
VSV (vesicular stomatitis virus),
Wallal virus,
Warrego virus,
wart virus,
WEE virus,
West Nile virus,
western equine encephalitis virus,
western equine encephalomyelitis virus,
Whataroa virus,
Winter Vomiting Virus,
woodchuck hepatitis B virus,
woolly monkey sarcoma virus,
wound tumor virus,
WRSV virus,
Yaba monkey tumor virus,
Yaba virus,
Yatapoxvirus,
yellow fever virus, and
Yug Bogdanovac virus, and . variants;
strains, and mutants of any virus listed, and
types, subtypes lineages, clades, and
subclades thereof.

### 11: Other Aspects

General Discussion

In this specification, stating a numerical range, it should be understood that all values within the range are also described (e.g., one to ten also includes every value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the disclosure or its patentability.

All modifications and substitutions that come within the meaning of the claims and the range of their legal equivalents are to be embraced within their scope. A claim which recites “comprising” allows the inclusion of other elements to be within the scope of the claim, the disclosure is also described by such claims reciting the transitional phrases “consisting essentially of” (i.e., allowing the inclusion of other elements to be within the scope of the claim if they do not materially affect operation of the disclosure) or “consisting of” (i.e., allowing only the elements listed in the claim other than impurities or inconsequential activities which are ordinarily associated with the disclosure) instead of the “comprising” term. Any of these three transitions can be used to claim the disclosure.

An element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed disclosure or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the disclosure. Similarly, generalizations of the disclosure's description are considered to be part of the disclosure.

From the foregoing, it would be apparent to a person of skill in this art that the disclosure can be embodied in other specific forms without departing from its spirit or essential characteristics.

While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

### INCORPORATION BY REFERENCE

All publications, patent applications, and patents mentioned herein are hereby incorporated by reference in their entirety as if each individual publication or patent was specifically and individually indicated to be incorporated by reference. These patents include, at least, U.S. Pat. Nos. 4,024,222, 4,130,641, 5,258,369, 7,439,349, 8,722,874 and 9,315,538. In case of conflict, the present application, including any definitions herein, will control.

### Examples

### Example 1: tdsRNA Synthesis and Purification

Strand synthesis was performed by polymerization of ribonucleotide diphosphates using polynucleotide phosphorylase (PNPase) made from M. luteus bacteria. PNPase catalyzes both processive 3′→5′ phosphorolysis and 5′→3′ polymerization of RNA. Unlike RNA polymerases, PNPase neither requires a template nor can transcribe one. When a mixture of ribonucleotide diphosphates (NDPs) serves as the substrate for the polymerization reaction, the ensuing polymerization reaction forms a random copolymer. Conversely, when only one type of ribonucleotide diphosphate was present as the substrate for the polymerization reaction, the product will be a polymer of a single type of subunit.

Synthesis of the first ssRNA (referred to herein as the first synthesis reaction) was performed in purified water and with PNPase with the following ingredients: ribonucleotide diphosphate 22 mM; Tris(hydroxymethyl)aminomethane 100 mM; MgCl₂ 5 mM; EDTA 1 mM; Urea 300 mM.

Where ssRNA with the structure of rIₙ was desired, the ribonucleotide diphosphate poly(I) at the prescribed concentration of 22 mM was the only ribonucleotide diphosphate present in the reaction.

Synthesis of the second ssRNA (referred to herein as the second synthesis reaction) was performed as follows. In the case where ssRNA comprising C and U subunits was desired, the reaction was performed in purified water and with PNPase with the following ingredients: ribonucleotide diphosphates 22 mM total—comprising a mixture of rCDP and rUDP, where a ratio of (molar concentration of rCDP)/(molar concentration of rUDP) was 12; Tris(hydroxymethyl)aminomethane 100 mM; MgCl₂ 5 mM; EDTA 1 mM; Urea 300 mM.

Where a ssRNA with the structure of r(C₁₂U)ₙ for example was desired, the ribonucleotide diphosphates rCDP and rUDP at a molar ratio of rCDP/rUDP=12 and a total concentration of 22 mM are the only ribonucleotide diphosphate present in the reaction.

The first synthesis reaction and the second synthesis reaction were performed in separate reactions and in separate vessels. Naturally, the first synthesis reaction and the second synthesis reaction can be performed in any order. Reaction was started by the addition of PNPase as the sole RNA polymerase in the range of 500-700 Units per Liter of reaction. The reaction temperature was maintained at 21° C.-25° C. for 12 to 48 hours. All of the synthesis reactions were performed in the absence of any ATP, in the absence of DNA components (i.e., DNA, DNA templates, dNTPs, dNDPs, dNMPs), and in the absence of any ribo bases that are not desired to be incorporated into the ssRNA chain. For example, if the first synthesis reaction was designed to synthesize rIₙ, then the synthesis reaction was performed in the absence of rATP, rADP, rAMP, rUTP, rUDP, rUMP, rGTP, rGDP, rGMP, rCTP, rCDP, rCMP, rITP, and rIMP.

That was because only rIDP was needed, rIDP was the only nucleotide in the reaction. As another example, if the second synthesis reaction was designed to synthesize r(C₁₂U)ₙ, then the synthesis reaction was performed in the absence of rATP, rADP, rAMP, rUTP, rUMP, rGTP, rGDP, rGMP, rCTP, rCMP, rITP, rIDP and rIMP.

That was because only rCDP and rUDP was needed.

RNA chain elongation was stopped by adding EDTA-Na₂ until EDTA-Na₂ concentration reached 26.9 mM.

Synthesized ssRNA from the first or the second synthesis reaction were purified as follows. Purification comprises four phenol extractions in the presence of SDS and Tris. The “phenol” used in phenol extraction was a composition of phenol: chloroform: isoamyl alcohol (25:24:1) at a pH of between 4-6. As a substitute, acid guanidinium thiocyanate-phenol-chloroform extraction may be used instead of phenol extraction. After each phenol extraction, the aqueous layer was collected and after the final extraction, the aqueous solution was precipitated in alcohol at a KCl concentration of 450 mM—achieved by the addition of KCl. The resulting precipitate was dissolved in water, and precipitated again in alcohol with 450 mM KCl. The precipitate was dissolved in water forming a solution of ssRNA. 10 mM EDTA-K2 was added to substitute the K⁺ ions for the Na⁺ ions. EDTA-K2 prevents the degradation of RNA and functions as an RNase inhibitor. This solution was concentrated and dialyzed against 7 volumes of water with 10 mM KC₂H₃O₂ to remove the salts and ribonucleotide diphosphates, thus producing an aqueous solution containing single-stranded RNA. This solution was filtered through a 0.22 um filter.

The aqueous single-stranded RNA solutions are brought to a final concentration of 7.9 mM in sodium phosphate buffer (150 mM NaCl; 1 mM MgCl₂; 8 mM Na₂HPO₄; 1.6 mM NaH₂PO₄). Equivalent volumes of the two single-stranded RNA solutions, at equivalent molar concentrations of first single-stranded RNA and second single-stranded RNA were mixed by slow addition of one solution to a second solution with stirring for at least 5 minutes. The mixed solution was heated to 65° C. for up to 30 minutes and then to 50° C. over 30 minutes.

The solution was then filtered by a 0.22 micron sterile filter to produce tdsRNA (a dsRNA). Products of the most desirable quality were produced when the above protocol was performed sequentially without interruption or intermediate storage steps such as lyophilization, drying, resuspension etc. between steps to minimize any changes to the dsRNA.

The above process has been repeated multiple times with consistent results and produces a tdsRNA which is further described in this disclosure. Rintatolimod in all the examples of this disclosure refers to rintatolimod made using the process of this example. Further, while the above disclosed method has been disclosed for one formula or some formulas of tdsRNA, all the formulas of tdsRNA may be made using this disclosed method.

### Example 2: Detecting SARS-CoV-2 Virus

The nucleic acid sequence and protein sequence of SARS-CoV-2 is known and published. See, e.g., the sequence listing section where SARS-CoV-2 was initially called “Wuhan seafood market pneumonia virus.” The detection of the SARS-CoV-2 virus by detecting its nucleic acid by reverse-transcription polymerase chain reaction (RT-PCR) and by detecting its protein has been performed and published by others. The protein and nucleic acid sequences is found, for example, in Genbank as follows: (1) Wuhan seafood market pneumonia virus isolate Wuhan-Hu-1, complete genome; (2) GenBank: MN908947.3; (3) LOCUS MN908947 29903 bp ss-RNA linear VRL 23 Jan. 2020; (4) ACCESSION MN908947; (5) VERSION MN908947.3.

A number of primers that can be successfully used in PCR or RT-PCR for detecting SARS-CoV-2 and monitoring SARS-CoV-2 titer and infectious status are also in the sequence listing section. Therefore, one can detect SARS-CoV-2 by collecting tissue samples from a subject and performing PCR or RT-PCR using primers.

Monitoring SARS-CoV-2 titer may involve determining the level (e.g., concentration, amount) of SARS-CoV-2 nucleic acid in a sample. Monitoring SARS-CoV-2 infection (SARS-CoV-2 infection) status may involve collecting tissue samples such as lung, nasal, blood, oral samples, or body fluid from a subject and performing a test such as PCR or RT-PCR using PCR primers (e.g., from the sequence listing section of this disclosure).

### Example 3: TdsRNA Activity Against SARS-CoV

tdsRNA (Rintatolimod) has in vivo antiviral activity against the pathogenesis of SARS-CoV which has highly lethal effects in humans as well as in BALB/c mice. This mouse model largely mimics the human disease and causes a lethal pulmonary syndrome in 5-6 week old BALB/c mice. TdsRNA at 10 mg/kg/day produced complete protection against death (10/10 mice survived compared to 0/10 mice surviving in the saline control group). TdsRNA also significantly reduced lung scores and weight loss (p<0.05).

Table 3 summarizes the findings of nine published studies of the activity of Rintatolimod, poly ICLC, and Ribavirin in SARS models. In addition, one recombinant human hybrid interferon (Interferon B/D) which is active in mice is also included. Individual in vitro drug activity data available from each publication is presented in terms of IC50 (50% Inhibitory Concentration) and EC50 (50% Effective Concentration) as represented by the respective authors, and is based on achievable drug serum levels relative to IC50 or EC50 drug concentrations. To be considered active against SARS at clinically achievable serum levels, the maximum achievable concentration (Cmax) in humans had to be higher than the IC50 or EC50.

In two studies (Barnard 2006 and Day 2009), SARS mouse models were evaluated and the drugs utilized in these studies had to yield their clinical effect, i.e., reduction of SARS virus lung titers below the level of detection (Barnard 2006) or increased survival (Day 2009) at clinically achievable/tolerated human dosage levels in order to be considered active. Only one drug, Rintatolimod, was active at clinically achievable serum/dosage levels. Barnard (2003) studied the inhibition of human CoV strain OC43 cytopathic effect (CPE) using Rintatolimod, poly ICLC, and Ribavirin. Rintatolimod had an EC50 of 0.4 μg/ml, while 50 μg/ml is achievable in human serum at dosages of 400 mg, which is utilized clinically (Strayer 2012). Barnard (2006) studied the ability of Rintatolimod and rIFN-αB/D to inhibit SARS virus (Urbani Strain) titers in the lungs of BALB/c mice. Complete inhibition to below detectable levels was seen with Rintatolimod at 10 mg/kg which is equivalent to 700 mg (in an average weight human (70 kg)) and has also been shown to be a generally well-tolerated dosage level (Thompson 1996). The rIFN-α B/D inhibited virus titers to below detectable level, but required a dose of 100,000 IU in mice, which is equivalent to an extremely high human dose of 500×10⁶ IU, and furthermore is not clinically available. Day (2009) studied a new mouse adapted strain of SARS-CoV as a lethal model. Only Rintatolimod at a dosage of 10 mg/kg/day obtained a 100% survival rate. In contrast, Ribavirin required a human dosage equivalent of 4,500 mg/day, which is over three times greater than the highest recommended dose of 1,200 mg/day in order to obtain a 30% survival rate. Ribavirin was studied in six other publications and was uniformly not active at clinically achievable serum concentrations (3.7 μg/ml).

Finally, Lee (1983) studied the activity of Rintatolimod against the mouse hepatitis coronavirus (MHV). Rintatolimod demonstrated both prophylactic and therapeutic activity with increased survival at dosage levels (10 μg/dose) that are clinically achievable in humans.

TABLE 3
Activity of Rintatolimod, Poly ICLC, and Ribavirin in SARS-CoV
Models

| | | | | |
|---|---|---|---|---|
| | | | | Clinically |
| | | SARS/CoV | Activity | Achievable |
| | In vivo | Model Virus | Measurement/ | Serum or |
| Reference/ | or | Strain/Dose | Drug Dose or | Dosage |
| Compound | In vitro | Route | Concentration | Levels |

Barnard (2003)¹

| | | | | |
|---|---|---|---|---|
| Rintatolimod | In vitro | Strain OC43/ | EC50: 0.4 | Yes⁹ |
| | | BS-C-1 Kidney | μg/mL | |
| Poly ICLC | | cells (African | EC50: >100 | No¹⁰ |
| | | Green Monkey) | μg/mL | |
| Ribavirin | | | EC50: 125 | No¹¹ |
| | | | μg/mL | |

Barnard (2006)²

| | | | | |
|---|---|---|---|---|
| Rintatolimod | In vivo | Urbani strain/ | 10 mg/kg | Yes¹² |
| | | BALB/c mice/ | | |
| | | Reduction of | | |
| Interferon B/D | | virus lung titers | 100,000 | No* |
| (Recombinant | | below level | IU/mouse | |
| cross species | | of detection | | |
| hybrid) | | on Day 3/IP | | |

Day (2009)³

| | | | | |
|---|---|---|---|---|
| Rintatolimod | In vivo | Strain v2163/ | 100% | Yes¹² |
| | | BALB/c mice/ | Survival | |
| | | Survival/IP | (10/10) | |
| | | | P < 0.001 | |
| | | | 10 mg/kg/day | |
| Ribavirin | | | 30% Survival | No¹¹ |
| | | | (3/10) 65 | |
| | | | mg/kg/day | |

Tan (2004)⁴

| | | | | |
|---|---|---|---|---|
| Ribavirin | In vitro | 2003VA2774/ | IC100: 5,000 | No¹¹ |
| | | Vero E6 Kidney | μg/mL | |
| | | cells (African | | |
| | | Green Monkey) | | |

Lee (1983)⁵

| | | | | |
|---|---|---|---|---|
| Rintatolimod | In vivo | Mouse Hepatitis | Increased | Yes¹² |
| | | CoV MHV/ | survival 10 | |
| | | BALB/c mice/ | μg/dose | |
| | | Survival/IP | | |

Chen (2004)⁶

| | | | | |
|---|---|---|---|---|
| Ribavirin | In vitro | Isolates from | EC50: 12-200 | No¹¹ |
| | | patients/fRhK4 | μg/mL | |
| | | | EC50: 50-200 | |
| | | | μg/mL | |

Chen (2004)⁶

| | | | | |
|---|---|---|---|---|
| Ribavirin | In vitro | Strain 39849/ | EC50: 50-100 | No¹¹ |
| | | Vero E6 | μg/mL | |

Morgenstern (2005)⁷

| | | | | |
|---|---|---|---|---|
| Ribavirin | In vitro | Strain FFM1/ | EC50: 4.7 | No¹¹ |
| | | Caco-2 | μg/mL | |
| Ribavirin | In vitro | Strain FFM1/ | EC50: 5.3 | No¹¹ |
| | | CL-14 | μg/mL | |

Stroher (2004)⁸

| | | | | |
|---|---|---|---|---|
| Ribavirin | In vitro | Patient Isolate/ | No effect at | No¹¹ |
| | | Vero E6 | 2,000 μg/mL | |

*This compound is not clinically available.¹Barnard D L, Hubbard V D, Smee D F, Sidwell R W. Inhibition of human coronavirus by antiviral agents: Potential therapies for SARS coronavirus. 16th International Conference on Antiviral Research, Savannah GA. (2003) Abstract # Late Breaker 4.
²Barnard D L, Day C W, Bailey K, Heiner M, Montgomery R, Lauridsen L, Chan P K, Sidwell R W. Evaluation of immunomodulators, interferons and known in vitro SARS-CoV inhibitors for inhibition of SARS-CoV replication in BALB/c mice. Antivir Chern Chemother (2006) 17(5):275-84. Alferon was also tested in this model but since it is species-specific to human and non-human primates was not active.
³Day C W, Baric R, Cai S X, Frieman M, Kumakia Y, Morrey J D, Smee D F, and Barnard D L. A new mouse-adapted strain of SARS-CoV as a lethal model for evaluating antiviral agents in vitro and in vivo. Virology (2009) 395(2):210-222. doi: 10.1016/j.virol.2009.09.023.
⁴Tan E L C, Ooi E E, Lin C-Y, Tan H C, Ling A E, Lim B, et al. Inhibition of SARS coronavirus infection in vitro with clinically approved antiviral drugs. Emerg Infect Dis (2004) Apr. Available from: http://www.cde.gov/ncidod/EID/vol10no4/03-0458 .htm.
⁵Lee N H, Wilson S, Fung L F, Fisher N M, and Levy G A. The Effect of an Interferon Inducer on Mouse Hepatitis Virus Infection in Mice. Hepatology (1983)3(5):837.⁶Chen F, Chan K H, Jiang Y, Kao R Y T, Lu H T, Fan K W, Cheng V C C, Tsui W H W, Hung I F N, Lee T S W, Guan Y, Peiris J S M, Yuen K Y. In vitro susceptibility of 10 clinical isolates of SARS coronavirus to selected antiviral compounds. J Clin Virol (2004) 31:69-75.
⁷Morgenstern B, Michaelis M, Baerb P C, Doerra H W. Ribavirin and interferon-b synergistically inhibit SARS-associated coronavirus replication in animal and human cell lines. Biochem Biophys Res Comm (2005) 326:905-908.
⁸Stroher U, DiCaro A, Li Y, Strong J E, Aoki F, Plummer F, Jones S M and Feldmann H. Severe Acute Respiratory Syndrome—Related Coronavirus Is Inhibited by Interferon-a. J Infect Dis (2004) 189:1164-1167.⁹Strayer D R, Carter W, Strauss K T, Brodsky I, Suhadolnik R J, Ablashi D, et al. Long term improvements in Patients with Chronic Fatigue Syndrome Treated with Ampligen. J CFS (1995) 1:35-53.¹⁰Levine A, Sivulich M, Wiernik P, and Levy H. Initial Clinical Trials in Cancer Patients of Polyribosinic-Polyribocytidylic Acid Stabilized with Poly-L-lysine, in Carboxymethylcellulose {Poly(ICLC)}, a Highly Effective Interferon Inducer. Cancer Res (1979) 39:1645-1650.
¹¹Physicians’ Desk Reference (2003) Edition 57, page 3072
¹²Thompson K A, Strayer D R, Salvato P D, Thompson C E, Klimas N, Molavi A, et al. Results of a Double-Blinded Placebo-Controlled Study of the Double-Stranded RNA Drug PolyI:PolyC₁₂U in the Treatment of HIV Infection. Eur J Clin Microbiol Infect Dis (1996) 15:580-587.

### Example 4: Analysis of Genetic Similarities Between SARS-CoV and SARS-CoV-2

We further analyzed the similarity between SARS-CoV and SARS-CoV-2 and, to our surprise, found significant similarities with the results listed in the following paragraph. These surprising similarities among the analyzed sequences allow us to extend the functional treatment effects of tdsRNA to that of SARS-CoV-2. That is, tdsRNA of this disclosure has a therapeutic effect on SARS-CoV-2. This therapeutic effect is effective for both treatment and for prophylaxis (i.e., prevention).

TABLE 4
COMPARISON OF THE TRS¹ OF THE FIRST FIVE
ORFS OF SARS-COV VS. WUHAN-HU-1-COV (WUHAN-
HU-1-COV IS AN ISOLATE OF SARS-COV-2)

| CoV | Base | ORF | TRS Sequence |
|---|---|---|---|
| SARS (Tor2) | 60 | Leader | UCUCUAAACGAACUUUAA |
| Wuhan-HU-1 | 63 | | AAUCUGUG |
| | | | (SEQ ID. NO: 13) |
| | | | UCUCUAAACGAACUUUAA |
| | | | AAUCUGUG |
| | | | (SEQ ID. NO: 14) |
| SARS (Tor2) | 21,479 | S | CAACUAAACGAAC |
| Wuhan-HU-1 | | (spike) | (SEQ ID. NO: 15) |
| | 21,549 | | CAACUAAACGAAC |
| | | | (SEQ ID. NO: 16) |
| SARS (Tor2) | 25,252 | ORF3 | CACAUAAACGAACUU |
| Wuhan-HU-1 | | | (SEQ ID. NO: 17) |
| | 25,378 | | CACAUAAACGAACUU |
| | | | (SEQ ID. NO: 18) |
| SARS (Tor2) | 26,104 | Envelope | UGAGUACGAACUU |
| Wuhan-HU-1 | | | (SEQ ID. NO: 19) |
| | 26,232 | | UGAGUACGAACUU |
| | | | (SEQ ID. NO: 20) |
| SARS (Tor2) | 26,341 | M | GGUCUAAACGAACUAACU |
| Wuhan-HU-1 | | | (SEQ ID. NO: 21) |
| | 26,466 | | GGUCUAAACGAACUAAAU |
| | | | (SEQ ID. NO: 22) |

¹Marra, et al. Science, 300 (5624) pp. 1399-1404 (2003)

Table 4 shows a comparison of TRS (transcription regulatory sequences) for the first five open-reading frames (ORFs) for the SAR-CoV (Tor2) compared to the Wuhan-HU-1 virus recently isolated from a patient with a SARs-like illness in China. That is, Wuhan-HU-1-CoV is an isolate of SARS-CoV-2. While the SARS-CoV genome contains 29,751 nucleotides the Wuhan-HU-1 genome contains an additional 153 nucleotides for a total of 29,904. In the Wuhan-HU-1 virus, the first base of each ORF is shifted to a higher position.

Unexpectedly, four of the five TRS sequences (Leader, S (spike), ORFS, and Envelope) shown in Table 4 are identical for the SARS-CoV compared to the Wuhan-HU-1 isolate. The M protein has a single nucleotide change: the A at position 26,357 in the SARS-CoV is changed to a C in position 26,482 of the Wuhan-HU-1. Thus, the first five ORF TRS sequences of these two coronaviruses are almost identical with 84/85 nucleotides the same (99% homology).

A comparison of the entire genomes of the SAR-CoV and the Wuhan-HU-1 virus shows 81% homology further indicating the closeness of the Wuhan-HU-1 to the SAR-CoV and the usefulness of tdsRNA for the treatment and prophylaxis of this illness (Table 5).

TABLE 5
SEQUENCE PERCENT HOMOLOGY¹
BETWEEN CORONAVIRUSES

| | | Percent Homology | | | | | | |
|---|---|---|---|---|---|---|---|---|
| Code | Coronavirus | A | B | C | D | E | F | G |
| A | Wuhan 2019 | — | 81 | 58 | 52 | 50 | 49 | 53 |
| B | SARS | 81 | — | 57 | 51 | 49 | 49 | 52 |
| C | MERS | 58 | 57 | — | 52 | 49 | 48 | 52 |
| D | MHV CoV | 52 | 51 | 52 | — | 53 | 52 | 76 |
| E | HU CoV NL63 | 50 | 49 | 49 | 53 | — | 70 | 54 |
| F | HU CoV 229E | 49 | 49 | 48 | 52 | 70 | — | 53 |
| G | HU CoV OC43E | 53 | 52 | 52 | 76 | 54 | 53 | — |

¹Percent homology as defined by (1-Uncorrected Pairwise Distance) times 100

Another important pathogenic feature of the SARS-CoV and the Wuhan SARS-CoV-2 is that they both utilize the same ACE2 receptor to bind to an infect human cells.

This similarity along with the similarity of the analyzed sequence allows us to show, for the first time that the functional treatment effects of tdsRNA can be extended to the Wuhan Coronavirus (i.e., SARS-CoV-2). That is, tdsRNA of this disclosure has an effect to treat; to prevent; to attenuate; and any combination thereof; a SARS-CoV-2 infection (SARS-CoV-2 infection).

### Example 5: Intranasal and Oral Rintatolimod Administration Protocol Schema

Study Title: A Phase I/II Study to Evaluate the Safety and Activity of Rintatolimod (Poly I:Poly C₁₂U) for the Prevention and Treatment of Coronavirus Disease-2019 (COVID-19)
Design: 3 weeks of open-label treatment. If treatment is tolerated well, we may continue beyond 3 weeks. Followed by a follow-up phone call 30±3 days after last dose of study medication.
Population: Up to 40 volunteers at high risk of contracting the COVID-19 disease (i.e., personnel in hospitals, nursing homes and quarantine centers with direct contact with patients with COVID-19 including adult family members quarantined with a SARS-COV-2 infected individual). A concurrent control group of up to 40 personnel that are not receiving Rintatolimod, in the same hospital or quarantine centers, with direct contact with COVID-19 positive patients, will be followed for development of symptoms of COVID-19 using best standard of care.

### Primary Endpoints:

1. Safety (frequency of grade 3 or 4 adverse events considered probably or definitely related to study medication)
2. Activity
   a. Time to positive nasal swab SARS-COV-2 test
   b. Time to development of COVID-19 symptoms (fever >100.4° F., cough, fatigue, nasal congestion, shortness of breath, etc.)

### Secondary Endpoints, if Hospitalized:

1. Time to required use of supplemental oxygen.
2. Time to use of ventilator to assist with breathing.
3. Time to hospital discharge or death.
Regimen: Up to 40 patients to receive Rintatolimod administered intranasally and orally.

Intranasal Administration (AccuSpray Syringes):
Rintatolimod 1250 μg (250 μL/syringe) every other day for 21 days.
Each patient receives the 250 μL of Rintatolimod in one AccuSpray syringe in each nostril (500 μL all together for two sprayers per dose).

Oral Administration (Tuberculin Syringe):
Rintatolimod 2500 μg (1000 μL/syringe) every other day for 21 days.
Each patient receives the 1000 μL of Rintatolimod in one tuberculin syringe per dose. (Patients will be instructed not to swallow medication immediately, but to hold it in the mouth for at least 2 minutes while swishing it around in order to repeatedly cover all possible oral surfaces).
Rationale: The rational for dosing Rintatolimod orally and intranasally every other day (qod) is based on establishing an antiviral state in the nose, mouth, nasal pharynx and oral pharynx that will inhibit the replication of the SARS-COV-2 virus for at least 1-2 days (i.e., until the next dose). The route of human infection is believed to be primarily by a nasal or oral route. By dosing Rintatolimod qod both orally and intranasally, it is believed that SARS-COV-2 will be inhibited at the point of entry, and thus will be much less likely to cause a pulmonary infection.

### Inclusion Criteria:

1. Negative test of nasal swab for SARS-COV-2 by a government approved test/kit for the local jurisdiction.
2. Age Range: >18 years old
3. Serum creatinine ≤1.5 ULN; serum bilirubin ≤1.5 ULN.
4. Total WBC ≥3000/mm³, platelet count ≥100,000/mm³ and granulocytes ≥1500 mm³.
5. Hemoglobin >10.0 g/dl.
6. ALT and AST <2 times upper normal limit.
7. Males or non-pregnant, non-lactating females: Females must be of non-child-bearing potential (either post-menopausal for two years or surgically sterile including tubal ligation) or using an effective means of contraception (birth control pills, intrauterine device, diaphragm). Females who are less than two (2) years post-menopausal, those with tubal ligations and those using contraception must have a negative urine pregnancy test prior to the first study medication administration. At study termination a pregnancy test should be performed, either serum or urine stick test. However, if the urine result is positive, a serum pregnancy test will be performed. Females of child-bearing potential agree to use an effective means of contraception during the study and up to four (4) weeks after the last study medication administration.
8. Ability to provide written informed consent indicating a willingness to participate in this investigational study.

### Exclusion Criteria:

1. Evidence of HIV or other viral infections including chronic hepatitis, or other disorders that would limit the subject's ability to complete the study.
2. Evidence of any autoimmune disorder.
3. Evidence of decompensated liver disease.
4. Cardiac risk factors including:
   Patients experiencing cardiac event(s) (acute coronary syndrome, myocardial infarction, or ischemia) within past 3 months.
   Patients with a New York Heart Association classification of III or IV
5. Patients with known serious mood disorders.
6. Unlikely or unable to comply with the requirements of the protocol.
7. Patients unwilling or unable to give informed consent.
8. Patients on any other concurrent experimental medication.
9. Therapy with interferons, interleukins, or other cytokines or investigational drugs within 6 weeks of beginning study medication. Subjects must give written informed consent prior to discontinuation of excluded drugs.
10. Any additional condition which in the Investigator's opinion deems the participant an unsuitable candidate to receive the study drug.

### Schedule of Tests and Examinations:

| | | | | | | | | | | | | | Follow-up |
|---|---|---|---|---|---|---|---|---|---|---|---|---|---|
| | Day | Day | Day | Day | Day | Day | Day | Day | Day | Day | Day | | on Day |
| | 1 | 3 | 5 | 7 | 9 | 11 | 13 | 15 | 17 | 19 | 21 | Days >21⁷ | 30 ± 3⁸ |
| History & | X | | | X | | | X | | | | X | q8d | |
| Physical Exam¹ | | | | | | | | | | | | | |
| Vital Signs² | X | X | X | X | X | X | X | X | X | X | X | qod | |
| Urine | X | | | | | | | | | | | EOT | |
| Pregnancy Test | | | | | | | | | | | | | |
| (if applicable) | | | | | | | | | | | | | |
| Chemistry | X | | | X | | | X | | | | X | q8d | |
| Panel³ | | | | | | | | | | | | | |
| CBC, Diff, | X | | | X | | | X | | | | X | q8d | |
| Platelets | | | | | | | | | | | | | |
| Urinalysis | X | | | X | | | X | | | | X | q8d | |
| Coagulation | X | | | X | | | X | | | | X | q8d | |
| Panel | | | | | | | | | | | | | |
| Nasal Swab | X | | | X | | | X | | | | X | q8d | |
| Samples | | | | | | | | | | | | | |
| Stored Serum | X | | | X | | | X | | | | X | q8d | |
| Samples^{4,5} | | | | | | | | | | | | | |
| (including | | | | | | | | | | | | | |
| SARS-CoV-2 | | | | | | | | | | | | | |
| antibody) | | | | | | | | | | | | | |
| Evaluation of | X | X | X | X | X | X | X | X | X | X | X | qod | X |
| COVID-19 | | | | | | | | | | | | | |
| symptoms | | | | | | | | | | | | | |
| Adverse Events | X | X | X | X | X | X | X | X | X | X | X | qod | X |
| Rintatolimod | X | X | X | X | X | X | X | X | X | X | X | qod | |
| Administration⁶ | | | | | | | | | | | | | |
| Telephone | | | | | | | | | | | | | X |
| Follow-up | | | | | | | | | | | | | |

ALL ASSESSMENTS TO BE PERFORMED PRIOR TO RINTATOLIMOD DOSING.
¹A medical history and physical exam (including weight) will be obtained on Day 1 (prior to Rintatolimod dosing). Interim physical exam (including weight) will be obtained on Days 7, 13, 21, and every 8 days thereafter,
²Vital signs will be obtained before and 15 minutes after receiving each Rintatolimod dose
³The chemistry panel will consist of serum creatinine, electrolytes, calcium, phosphate, glucose, blood urea nitrogen, uric acid, cholesterol, total protein, albumin, bilirubin [total], alkaline phosphatase, lactic dehydrogenase, SGOT (AST), SGPT (ALT), amylase, and a c-reactive protein serum level.
⁴The assay for SARS-CoV-2 antibodies will be performed on Stored Serum Samples.
⁵Whole blood (10 ml) will be collected and processed for the serum sample to be stored frozen in 1 mL aliquots.
⁶Ampligen dosing includes both nasal applications and oral administration.
⁶If urine pregnancy test is positive, perform a blood pregnancy test.
⁷qod = every other day, q8d = every 8 days; EOT = End of treatment
⁸A follow-up phone call will occur 30 ± 3 days after last dose of study medication.

### Rationale for the Use of Intranasal and Oral Administration of Rintatolimod for Prevention of COVID-19

The rationale for dosing Rintatolimod orally and intranasally every other day (qod) is based on establishing an antiviral state in the nose, mouth, nasal pharynx and oral pharynx that will inhibit the replication of the SARS-COV-2 virus for at least 1-2 days (i.e., until the next dose). The route of human infection is believed to be primarily by a nasal or oral route. By dosing Rintatolimod qod both orally and intranasally, it is believed that SARS-COV-2 will be inhibited at the point of entry, and thus will be much less likely to cause a pulmonary infection.

Rintatolimod, a synthetic double-stranded RNA (Poly I: Poly C₁₂U), is a well-defined selective Toll-like receptor 3 (TLR3) agonist in the induction of innate immune antiviral responses [1]. TLR3 is expressed in high concentration in human airway epithelial cells [2] oral epithelial cells, pharyngeal epithelial cells, and esophageal epithelial cells [3] and serves as a pathogen recognition receptor to stimulate the innate immune response against many respiratory pathogens [2] including coronaviruses such as SARS-CoV-2. The intranasal bioactivity and safety of Rintatolimod as a mucosal adjuvant when administered with influenza virus vaccine has been demonstrated in both mouse and monkey models. Mice co-administered H5N1 influenza vaccine (A/Vietnam/1194/2004) antigen (NIBRG14) and Rintatolimod intranasally showed a robust antigen specific IgA response in the nasal mucosa [4]. Mice vaccinated with Rintatolimod-adjuvanted NIBRG14 vaccine were also protected against challenge with homologous (A/Vietnam/1194/04) and heterologous (A/HK/483/97 and A/Indonesia/6/05) H5N1 influenza viruses [4]. The activity and safety of Rintatolimod as an intranasal vaccine adjuvant was also demonstrated in cynomolgus macaques [5]. Monkeys immunized with three doses of NIBRG14 vaccine and Rintatolimod were completely protected from challenge infection with the homologous virus. Vaccine specific salivary IgA and serum IgG antibodies were detected after the second immunization. In addition, when assayed 2 weeks after the third immunization, the immunized monkeys also developed serum neutralizing anti-body activity against both homologous (A/Vietnam/1194/04) and heterologous (A/HK/483/97 and A/Indonesia/6/05) H5N1 influenza virus. Moreover, intranasal administration of Rintatolimod with seasonal inactivated trivalent influenza vaccine (H1N1, H3N2, B) was able to protect mice from lethal challenge doses of three avian H5N1 influenza virus clades (A/Vietnam/1194/04, A/Hong Kong/483/97, and A/Indonesia/16/2005) which are highly pathogenic for humans [6].

Rintatolimod has the potential to serve as a potent and universal adjuvant for intranasally delivered vaccines. A study (AMP-600) was designed to assess the safety and immunogenicity of FluMist® intranasal influenza vaccine administration followed by intranasal Rintatolimod in humans. This study was designed to recapitulate the murine model assessing the impact of Rintatolimod to augment immune responses to intranasal trivalent seasonal influenza vaccine in healthy adults.

In the AMP-600 study using intranasal FluMist® vaccine and Rintatolimod as a vaccine enhancer, the Rintatolimod administration was given 3 days after the FluMist® since peak viral shedding in the nose is expected approximately 72 hours following FluMist® administration. With the dual role of Rintatolimod as an antiviral agent and immune enhancer, it was anticipated that Rintatolimod given 72 hours post FluMist® application would boost immunological response without having a significant impact on viral shedding. This approach worked in that the intranasal administration of the seasonal influenza vaccine followed by Rintatolimod induced cross-reactive IgA antibody formation against avian H5N1 and H7N9 influenza hemagglutinins (HAs) in the human volunteers. The combinational use of Rintatolimod plus FluMist® was well tolerated [7].

With the well-tolerated safety profile of Rintatolimod administered intranasally at a concentration of 2.5 mg/ml as demonstrated in both preclinical and clinical studies, Rintatolimod has the potential to serve as a potent and broad-spectrum antiviral for intranasal and oral administration.

Also, Rintatolimod has been generally well-tolerated in Chronic Fatigue Syndrome (CFS) patients treated in placebo-controlled trials when administered intravenously [8]. To date, over 100,000 intravenous doses have been administered.

The oral administration of Rintatolimod is intended to protect the oral cavity and oral pharynx which will be accomplished by swishing the Rintatolimod around in the mouth, prior to being swallowed. It is expected that the Rintatolimod will induce an antiviral response in the oral cavity and oral pharynx with the potential to greatly reduce the SARS-CoV-2 viral load entering the trachea and lungs.

Why would Rintatolimod be expected to induce an anti-viral state in the cavity? The oral cavity [3], like the nose and nasal pharynx [2], contains high levels of TLR3 receptors which are required for Rintatolimod to induce both innate and adaptive immune responses including antiviral responses. Thus, the well-tolerated safety profile and bio-activity of Rintatolimod administered intranasally is well documented in both animal models [4,5,6] and humans [7] and a similar bio-activity and safety profile is expected following oral administration.

### REFERENCES

[1] Gowen B B, Wong M-H, Jung K-H, Sanders A B, Mitchell W M, Alexopoulou L, et al. TLR3 is essential for the induction of protective immunity against Punta Toro virus infection by the double-stranded RNA (dsRNA), Poly(I:C12U), but not Poly(I:C): differential recognition of synthetic dsRNA molecules. J Immunol 2007; 178:5200-8.
[2] Hewson C A, Jardine A, Edwards M R, Laza-Stanca V, Johnston S L. Toll-like receptor 3 is induced by and mediates antiviral activity against rhinovirus infection of human bronchial epithelial cells. J Virol 2005; 79(19):12273-9.
[3] Uehara A, et al. Various human epithelial cells express functional Toll-like receptors, NOD1 and NOD2 to produce anti-microbial peptides, but not proinflammatory cytokines, Molecular Immunology 44 (2007) 3100-3111
[4] Ichinohe T, Kawaguchi A, Tamura S, Takahashi H, Sawa H, Ninomiya A, et al. Intranasal immunization with H5N1 vaccine plus Poly I: Poly C12U, a toll-like receptor agonist, protects mice against homologous and heterologous virus challenge. Microbes Infect 2007; 9:1333-40.
[5] Ichinohe T, Ainai A, Yasushi A, Nagata N, Iwata N, Kawaguchi A, et al. Intranasal administration of adjuvant-combined vaccine protects monkeys from challenge with the highly pathogenic influenza A H5N1 virus. J Med Virol 2010; 82:1754-61.
[6] Ichinohe T, Tamura S, Kawaguchi A, Imai M, Itamura S, Odagiri T, et al. Cross-protection against H5N1 influenza virus infection is afforded by intranasal inoculation with seasonal trivalent inactivated influenza vaccine. J Infect Dis 2007; 196:1313-20.
[7] Overton E T, et al. Intranasal seasonal influenza vaccine and a TLR-3 agonist, rintatolimod, increased cross-reactive IgA antibody formation against avian H5N1 and H7N9 influenza HA in humans. Vaccine 32 (2014) 5490-5495
[8] Strayer D R, Carter W A, Stouch B C, Stevens S R, Bateman L, Cimoch P J, et al. A double-blind, placebo-controlled, randomized, clinical trial of the TLR-3 agonist rintatolimod in severe cases of chronic fatigue syndrome. PLoS ONE 2012; 7(3):e31334.

### Example 6 Intravenous Rintatolimod or tdsRNA Administration Protocol Schema

Study Title: A Phase I/II Study to Evaluate the Safety and Activity of Rintatolimod (Poly I:Poly C₁₂U) in Patients with Early Onset Coronavirus Disease-2019 (COVID-19)
Design: Up to 4 weeks of open-label treatment followed by a follow-up phone call 30±3 days after last dose of study medication.
Population: Up to 40 patients who meet the following criteria.
   1. Positive nasal swab RT-PCR test for COVID-19
      a. If symptomatic, treatment initiated within 96 hours of COVID-19 symptom(s) onset.
      b. If asymptomatic, treatment initiated within 96 hours of positive SARS-COV-2 test.
   A concurrent control group of up to 40 patients who will not be receiving Rintatolimod, who would otherwise have qualified for the Rintatolimod infusions, will be followed using best standard of care.

### Primary Endpoints:

1. Safety (frequency of grade 3 or 4 adverse events considered probably or definitely related to study medication)
2. Activity (Time to negative nasal swab SARS-COV-2 test)

### Secondary Endpoints:

1. Time to resolution of COVID-19 symptomatology (if asymptomatic, time to initiation and resolution of symptoms)
2. Time to required use of supplemental oxygen
3. Time to use of ventilator to assist with breathing
4. Time to hospital discharge or death
Regimen: Patients will receive the best available standard of care in the treatment of their

COVID-19 and in addition will receive Rintatolimod.
Rintatolimod 200 mg IV twice weekly for 2 weeks beginning at Week 1 (4 doses)
Rintatolimod 400 mg IV twice weekly for 2 weeks beginning at Week 3 (4 doses) or until hospital discharge. All patients who remain hospitalized after 4 weeks will be followed on a weekly basis for safety and clinical recovery until discharge or death.

### Potential Synergistic Regimens:

After the safety profile of Rintatolimod administered by IV infusion is established in patients with COVID-19, the addition of other antiviral drugs with potential activity against SARS-COV-2 will be added as combinational therapeutic approaches. These drugs would include interferons (recombinant or natural), as Rintatolimod has demonstrated synergy with α, β and γ interferons (FIG. 1). Also, included would be small molecule antiviral drugs such as remdesivir. Rintatolimod has already demonstrated synergy with more than 12 different small molecule antiviral drugs such as remdesivir (FIG. 1). These additional antiviral drugs could be added to Rintatolimod as two drug or three drug or more combinational therapeutic approaches representing separate arms in the same or different studies.

### Inclusion Criteria:

1. Positive RT-PCR test of nasal swab for SARS-COV-2 by a government approved test/kit for the local jurisdiction.
2. Age Range: ≥18 years old.
3. For subjects symptomatic with regard to COVID-19 related clinical symptoms, the symptoms can include fever (>100.4° F.), fatigue, or respiratory symptoms of cough or nasal congestion, but cannot include shortness of breath or pulmonary infiltrates and subject must receive first dose within 96 hours of developing first symptom. If asymptomatic must be dosed within 96 hours from time nasal swab obtained that resulted in positive RT-PCR for COVID-19.
4. Serum creatinine ≤1.5 ULN; serum bilirubin ≤1.5 ULN.
5. Total WBC ≥3000/mm³, platelet count ≥100,000/mm³ and granulocytes ≥1500 mm³.
6. Hemoglobin >10.0 g/dl.
7. ALT and AST <2 times upper normal limit.
8. Males or non-pregnant, non-lactating females: Females must be of non-child-bearing potential (either post-menopausal for two years or surgically sterile including tubal ligation) or using an effective means of contraception (birth control pills, intrauterine device, diaphragm). Females who are less than two (2) years post-menopausal, those with tubal ligations and those using contraception must have a negative urine pregnancy test prior to the first study medication administration. At study termination a pregnancy test should be performed, either serum or urine stick test. However, if the urine result is positive, a serum pregnancy test will be performed. Females of child-bearing potential agree to use an effective means of contraception during the study and up to four (4) weeks after the last study medication administration.
9. Ability to provide written informed consent indicating a willingness to participate in this investigational study.

### Exclusion Criteria

1. Evidence of HIV or other viral infections including chronic hepatitis, or other disorders that would limit the subject's ability to complete the study.
2 Evidence of any autoimmune disorder.
3. Evidence of decompensated liver disease.
4. Cardiac risk factors including:
   Patients experiencing cardiac event(s) (acute coronary syndrome, myocardial infarction, or ischemia) within past 3 months.
   Patients with a New York Heart Association classification of III or IV
5. Patients with known serious mood disorders.
6. Unlikely or unable to comply with the requirements of the protocol.
7. Patients unwilling or unable to give informed consent.
8. Patients on any other concurrent experimental medication.
9. Therapy with interferons, interleukins, or other cytokines or investigational drugs within 6 weeks of beginning study medication. Subjects must give written informed consent prior to discontinuation of excluded drugs.
10. Any additional condition which in the Investigator's opinion deems the participant an unsuitable candidate to receive the study drug.

### Schedule of Tests and Examinations:

| | | | | | | | | | | | Follow- |
|---|---|---|---|---|---|---|---|---|---|---|---|
| | Week 1 | | Week 2 | | Week 3 | | Week 4 | | | | up |
| | Day | Day | Day | Day | Day | Day | Day | Day | Week(s) >4 | Day of | Day |
| | 1 | 4 | 8 | 11 | 15 | 18 | 22 | 25 | Weekly | Discharge | 30 ± 3⁷ |
| History & Physical Exam¹ | X | X | | X | | X | | X | X | X | |
| Vital Signs² | X | X | X | X | X | X | X | X | X | X | |
| Urine Pregnancy Test (if | X | | | | | | | | | X⁶ | |
| applicable) | | | | | | | | | | | |
| Chemistry Panel³ | X | X | | X | | X | | X | X | X | |
| CBC, Diff, Platelets | X | X | | X | | X | | X | X | X | |
| Urinalysis | X | X | | X | | X | | X | X | X | |
| Coagulation Panel | X | X | | X | | X | | X | X | X | |
| Nasal Swab Samples | X | X | X | X | X | X | X | X | X | X | |
| Stored Serum Samples^{4,5} | X | X | | X | | X | | X | X | X | |
| (including SARS-CoV-2 | | | | | | | | | | | |
| antibody) | | | | | | | | | | | |
| Evaluation of COVID-19 | X | X | X | X | X | X | X | X | X | X | X |
| symptoms | | | | | | | | | | | |
| Adverse Events | X | X | X | X | X | X | X | X | X | X | X |
| Rintatolimod Administration⁶ | X | X | X | X | X | X | X | X | | | |
| Telephone Follow-up | | | | | | | | | | | X |

All assessments to be performed prior to rintatolimod dosing.
¹A medical history and physical exam (including weight) will be obtained on Day 1 (prior to Rintatolimod dosing). Interim physical exam (including weight) will be obtained on Days 4, 11, 18, 25, weekly and on day of discharge.
²Vital signs will be obtained before and 30 minutes after receiving each Rintatolimod dose
³The chemistry panel will consist of serum creatinine, electrolytes, calcium, phosphate, glucose, blood urea nitrogen, uric acid, cholesterol, total protein, albumin, bilirubin [total], alkaline phosphatase, lactic dehydrogenase, SGOT (AST), SGPT (ALT), amylase, and a c-reactive protein serum level.
⁴The assay for SARS-CoV-2 antibodies will be performed on Stored Serum Samples.
⁵Whole blood (10 ml) will be collected and processed for the serum sample to be stored frozen in 1 mL aliquots.
⁶Ampligen dosing will be twice weekly for 4 weeks.
⁶If urine pregnancy test is positive, perform a blood pregnancy test.
⁷A follow-up phone call will occur 30 ± 3 days after last dose of study medication.

Experimental data (some unpublished) by us and which is also confirmed by experimental data by others have shown that rintatolimod enhances the efficacy of a broad spectrum of antiproliferative and antiviral drugs at least based on 3 lines of evidence: (1) Rintatolimod is synergistic with small molecule antiviral drugs as follows: Influenza (oseltamivir, zanamivir); HIV/AIDS2 (abacavir, zidovudine, zalcitabine, didanosine, stavudine, efavirenz, indinavir, ritonavir, nelfinavir, amprenavir, ribavirin). (2) Rintatolimod is synergistic with α, β, and γ interferons for Antiviral indication (HIV/AIDS). These interferons include rIFN-α-2a, rIFN-β-1b, rIFN-γ. (3) Rintatolimod is synergistic with α, β, and γ interferons for Antiproliferative (Cancer) indications: Interferons (nIFN-α, rIFN-α-2a, nIFN-β, rIFN-β-1b, nIFN-γ), Interleukins (IL-2), Checkpoint Inhibitor (Anti-PD-L1).

### Example 7 Intranasal Administration Protocol Schema

Study Title: A Phase I/II, Two-Stage, Randomized, Double-Blind Study to Evaluate the Safety and Activity of Rintatolimod (Poly I:Poly C1211) for the Prevention and Treatment of Coronavirus Disease-2019 (COVID-19)
Design: Phase I/II, two-staged, prospective, adaptive group sequential, randomized, double-blind study
   Treatment administered every other day for 28 days (4 weeks)
   All patients will be followed weekly for 28 days (4 weeks) after the last dose of study medication
Population: Initial target of 90 volunteers will be randomized into the study. Stage 1=30 volunteers; Stage 2=60 volunteers.
   All volunteers must be at high risk of contracting the COVID-19 disease (i.e., personnel in hospitals [such as healthcare workers], individuals in nursing homes or elder care facilities and in quarantine centers (such as cruise ships or Department of Defense ships) with possible direct contact with COVID-19 including adult family members quarantined with a SARS-COV-2 infected individual and first responders).

### Primary Endpoints:

1. Safety (frequency of grade 3 or 4 adverse events considered probably or definitely related to study medication)
2. Activity
   a. Time to laboratory confirmed SARS-COV-2 infection
   b. Time to development of COVID-19 symptoms (fever >100.4° F., cough, fatigue, nasal congestion, shortness of breath, etc.)

### Secondary Endpoints:

1. Time to required use of supplemental oxygen
2. Time to use of ventilator to assist with breathing
3. Time to hospital discharge or death

### Stage 1 Regimen:

Stage 1 will consist of 5 treatment groups

| | Group 1 | | Group 2 | | Group 3 | | Group 4 | | Group 5 | | Group 6 | |
|---|---|---|---|---|---|---|---|---|---|---|---|---|
| Number of Patients | 5 | | 5 | | 5 | | 5 | | 5 | | 5 | |
| Rintatolimod* | 25 | μg | 50 | μg | 100 | μg | 200 | μg | 500 | μg | 1250 | μg |
| | (n = 4) | | (n = 4) | | (n = 4) | | (n = 4) | | (n = 4) | | (n = 4) | |
| Placebo | 500 | μl | 500 | μl | 500 | μl | 500 | μl | 500 | μl | 500 | μl |

| | | | | | | |
|---|---|---|---|---|---|---|
| (saline)* | (n = 1) | (n = 1) | (n = 1) | (n = 1) | (n = 1) | (n = 1) |

*Each patient receives the 250 μL of Rintatolimod or placebo in one nasal sprayer in each nostril (500 μL all together for two sprayers per dose) every other day for 28 days.

Each Treatment Group will consist of 4 Rintatolimod patients and 1 placebo patient
Patients who terminate before Day 28 (week 4) for reasons other than toxicity related to the study drug will be replaced.

Stage 2 will consist of 2 treatment groups

| | Group 7 | Group 8 |
|---|---|---|
| Number of Patients | 30 | 30 |
| Rintatolimod | TBD* μg | |
| Placebo (saline) | | 500 μl |

*Rintatolimod dose to be determined from Stage 1 results

Patients who terminate before Day 28 (week 4) for reasons other than toxicity related to the study drug or development of severe COVID-19 will be replaced.
The initial target sample size of 60 evaluable subjects in Stage 2 will be enrolled in the study as part of this group sequential design. If the conditional power is >50% after 60 patients have completed the study the sample size for Stage 2 may be increased up to a maximum of 120 patients.
For any patient developing a positive test for COVID-19, Standard of Care for COVID-19 will be allowed. The standard of care is expected to change as additional information emerges.
Randomization:
   Patients will be randomized in a 4:1 ratio in Stage 1 and a 1:1 ratio in Stage 2.
Blinding:
   This is a double-blind study. The investigator and staff, patients and Sponsor/CRO staff directly related to study conduct will be blinded to the treatment assignment.
   A pharmacist will be responsible for preparation of the study medication and filling into the nasal sprayers; therefore, the pharmacist will be unblinded to treatment assignment.
Rationale: The rational for dosing Rintatolimod intranasally every other day (qod) is based on establishing an antiviral state in the nose, and nasal pharynx that will inhibit the replication of the SARS-COV-2 virus for at least 1-2 days (i.e., until the next dose). The route of human infection is believed to be primarily by a nasal or oral route. By dosing Rintatolimod qod intranasally, it is believed that SARS-COV-2 will be inhibited at the point of entry, and thus will be much less likely to cause a pulmonary infection, or moderate COVID-19 disease.

### Inclusion Criteria:

1. Laboratory confirmed negative SARS-COV-2 infection by a government approved test/kit for the local jurisdiction.
2. Age Range: >18 years old
3. Respiration Rate <20 and Heart Rate <90 bpm SpO2 ≥94% on room air.
5. Serum creatinine ≤2.0 ULN; serum bilirubin ≤2.0 ULN.
6. Total WBC ≥3000/mm³, platelet count ≥100,000/mm³ and granulocytes ≥1500 mm³.
7. Hemoglobin >10.0 g/dl.
8. ALT and AST <2.5 times upper normal limit.
9. Males or non-pregnant, non-lactating females: Females must be of non-child-bearing potential (either post-menopausal for two years or surgically sterile including tubal ligation) or using an effective means of contraception (birth control pills, intrauterine device, diaphragm). Females who are less than two (2) years post-menopausal, those with tubal ligations and those using contraception must have a negative urine pregnancy test prior to the first study medication administration. At study termination a pregnancy test should be performed, either serum or urine stick test. However, if the urine result is positive, a serum pregnancy test will be performed. Females of child-bearing potential agree to use an effective means of contraception during the study and up to four (4) weeks after the last study medication administration.
10. Ability to provide written informed consent indicating a willingness to participate in this investigational study.

### Exclusion Criteria:

1. Evidence of HIV or other viral infections including chronic hepatitis, or other disorders that would limit the subject's ability to complete the study.
2. Evidence of rales or crackles on physical exam.
3. Evidence of shortness of breath (SOB), or dyspnea.
4. Evidence of ARDS or SIRS/shock or cardiac failure.
5. Evidence of any autoimmune disorder.
6. Evidence of decompensated liver disease.
7. Cardiac risk factors including:
   Patients experiencing cardiac event(s) (acute coronary syndrome, myocardial infarction, or ischemia) within past 3 months.
   Patients with a New York Heart Association classification of III or IV
8. Patients with known serious mood disorders.
9. Patients with elevated inflammatory markers, such as CRP, LDH, D-dimer, ferritin, IL-6.
10. Patients with lymphopenia
11. Unlikely or unable to comply with the requirements of the protocol.
12. Patients unwilling or unable to give informed consent.
13. Patients on any other concurrent experimental medication.
14. Therapy with interferons, interleukins, or other cytokines or investigational drugs within 6 weeks of beginning study medication. Subjects must give written informed consent prior to discontinuation of excluded drugs.
15. Any additional condition which in the Investigator's opinion deems the participant an unsuitable candidate to receive the study drug.
Safety Review: The data monitoring committee (DMC), comprised of researchers independent of the sponsor and the trial, will assess the progress of the study, the safety data and activity of study medication. The DMC will review safety data (i.e., serious or severe adverse events definitely or possibly related to study drug) for each Treatment Group prior to initiation of the next Treatment Group in Stage 1. The DMC will review safety data (i.e., serious or severe adverse events definitely or possibly related to study drug) after completion of the first 10 patients in Stage 2; before proceeding with an enrollment of the remaining 50 patients. The DMC will provide recommendations for early termination or design adaptations based on unblinded interim analysis (i.e., completion of each Treatment Group in Stage 1 and after 10 patients complete treatment in Stage 2).

Schedule of Tests and Examinations:

| | | | | | | | | | | | | | | | | Weekly |
|---|---|---|---|---|---|---|---|---|---|---|---|---|---|---|---|---|
| | | | | | | | | | | | | | | | | for 28 |
| | Base- | Day | Day | Day | Day | Day | Day | Day | Day | Day | Day | Day | Day | Day | Day | Days (to |
| | line | 1 | 3 | 5 | 7 | 9 | 11 | 13 | 15 | 17 | 19 | 21 | 23 | 25 | 27 | Day 56) |
| History & Physical | X | X | | | X | | | X | | | | X | | | X | X |
| Exam¹ | | | | | | | | | | | | | | | | |
| Vital Signs² | X | X | X | X | X | X | X | X | X | X | X | X | X | X | X | X |
| Urine Pregnancy | X | | | | | | | | | | | | | | | |
| Test (if applicable) | | | | | | | | | | | | | | | | |
| Chemistry Panel³ | X | | | | X | | | X | | | | X | | | X | X |
| CBC, Diff, | X | | | | X | | | X | | | | X | | | X | X |
| Platelets | | | | | | | | | | | | | | | | |
| Urinalysis | X | | | | X | | | X | | | | X | | | X | X |
| Coagulation Panel | X | | | | X | | | X | | | | X | | | X | X |
| Nasal Swab | X | | | | X | | | X | | | | X | | | X | X |
| Samples | | | | | | | | | | | | | | | | |
| Stored Serum | X | | | | X | | | X | | | | X | | | X | X |
| Samples^{4,5} | | | | | | | | | | | | | | | | |
| Evaluation of | X | X | X | X | X | X | X | X | X | X | X | X | X | X | X | X |
| COVID-19 | | | | | | | | | | | | | | | | |
| symptoms | | | | | | | | | | | | | | | | |
| Adverse Events | X | X | X | X | X | X | X | X | X | X | X | X | X | X | X | X |
| Rintatolimod or | X | X | X | X | X | X | X | X | X | X | X | X | X | X | X | |
| Placebo | | | | | | | | | | | | | | | | |
| Administration | | | | | | | | | | | | | | | | |

ALL ASSESSMENTS TO BE PERFORMED PRIOR TO STUDY DRUG DOSING.
¹A medical history and physical exam (including weight) will be obtained on Day 1 (prior to Study Drug dosing). Interim physical exam (including weight) will be obtained on Days 7, 13, 21, 27, and weekly,
²Vital signs will be obtained before and 15 minutes after receiving each Rintatolimod dose
³The chemistry panel will consist of serum creatinine, electrolytes, calcium, phosphate, glucose, blood urea nitrogen, uric acid, cholesterol, total protein, albumin, bilirubin [total], alkaline phosphatase, lactic dehydrogenase, SGOT (AST), SGPT (ALT), amylase, and a c-reactive protein serum level.
⁴The assay for SARS-CoV-2 antibody testing will be performed on Stored Serum Samples.
⁵Whole blood (10 ml) will be collected and processed for the serum sample to be stored frozen in 1 mL aliquots.
⁶If urine pregnancy test is positive, perform a blood pregnancy test.

### Rationale for the Use of Intranasal Administration of Rintatolimod for Prevention of COVID-19

The rationale for dosing Rintatolimod intranasally every other day (qod) is based on establishing an antiviral state in the nose, mouth, and nasal pharynx that will inhibit the replication of the SARS-COV-2 virus for at least 1-2 days (i.e., until the next dose). The route of human infection is believed to be primarily by a nasal route. By dosing Rintatolimod qod intranasally, it is believed that SARS-COV-2 will be inhibited at the point of entry, and thus will be much less likely to cause a pulmonary infection, or moderate COVID-19 disease.

Rintatolimod, a synthetic double-stranded RNA (Poly I: Poly C12U), is a well-defined selective Toll-like receptor 3 (TLR3) agonist in the induction of innate immune antiviral responses [1]. TLR3 is expressed in high concentration in human airway epithelial cells [2] oral epithelial cells, pharyngeal epithelial cells, and esophageal epithelial cells [3] and serves as a pathogen recognition receptor to stimulate the innate immune response against many respiratory pathogens [2] including coronaviruses such as SARS-CoV-2. The intranasal bioactivity and safety of Rintatolimod as a mucosal adjuvant when administered with influenza virus vaccine has been demonstrated in both mouse and monkey models. Mice co-administered H5N1 influenza vaccine (A/Vietnam/1194/2004) antigen (NIBRG14) and Rintatolimod intranasally showed a robust antigen specific IgA response in the nasal mucosa [4]. Mice vaccinated with Rintatolimod-adjuvanted NIBRG14 vaccine were also protected against challenge with homologous (A/Vietnam/1194/04) and heterologous (A/HK/483/97 and A/Indonesia/6/05) H5N1 influenza viruses [4]. The activity and safety of Rintatolimod as an intranasal vaccine adjuvant was also demonstrated in cynomolgus macaques [5]. Monkeys immunized with three doses of NIBRG14 vaccine and Rintatolimod were completely protected from challenge infection with the homologous virus. Vaccine specific salivary IgA and serum IgG antibodies were detected after the second immunization. In addition, when assayed 2 weeks after the third immunization, the immunized monkeys also developed serum neutralizing anti-body activity against both homologous (A/Vietnam/1194/04) and heterologous (A/HK/483/97 and A/Indonesia/6/05) H5N1 influenza virus. Moreover, intranasal administration of Rintatolimod with seasonal inactivated trivalent influenza vaccine (H1N1, H3N2, B) was able to protect mice from lethal challenge doses of three avian H5N1 influenza virus clades (A/Vietnam/1194/04, A/Hong Kong/483/97, and A/Indonesia/16/2005) which are highly pathogenic for humans [6].

Rintatolimod has the potential to serve as a potent and universal adjuvant for intranasally delivered vaccines. A study (AMP-600) was designed to assess the safety and immunogenicity of FluMist® intranasal influenza vaccine administration followed by intranasal Rintatolimod in humans. This study was designed to recapitulate the murine model assessing the impact of Rintatolimod to augment immune responses to intranasal trivalent seasonal influenza vaccine in healthy adults.

In the AMP-600 study using intranasal Flumist vaccine and Rintatolimod as a vaccine enhancer, the Rintatolimod administration was given 3 days after the Flumist since peak viral shedding in the nose is expected approximately 72 hours following Flumist administration. With the dual role of Rintatolimod as an antiviral agent and immune enhancer, it was anticipated that Rintatolimod given 72 hours post Flumist application would boost immunological response without having a significant impact on viral shedding. This approach worked in that the intranasal administration of the seasonal influenza vaccine followed by Rintatolimod induced cross-reactive IgA antibody formation against avian H5N1 and H7N9 influenza hemagglutinins (HAs) in the human volunteers. The combinational use of Rintatolimod plus Flumist was well tolerated [7].

With the well-tolerated safety profile of Rintatolimod administered intranasally at a concentration of 2.5 mg/ml as demonstrated in both preclinical and clinical studies, Rintatolimod has the potential to serve as a potent and broad-spectrum antiviral for intranasal administration.

Also, Rintatolimod has been generally well-tolerated in Chronic Fatigue Syndrome (CFS) patients treated in placebo-controlled trials when administered intravenously [8]. To date, over 100,000 intravenous doses have been administered

Why would Rintatolimod be expected to induce an anti-viral state in the nasal cavity with the potential to block COVID-19 systemic disease? The nose and nasal pharynx [2] contains high levels of TLR3 receptors which are required for Rintatolimod to induce both innate and adaptive immune responses including antiviral responses. In the years that followed the first SARS-CoV-1 epidemic, studies of SARS-CoV-1 in mouse models showed that Rintatolimod demonstrated a protective effect. Barnard 2006 [9] showed that Rintatolimod “reduced virus lung titers to below detectable limits.” Day 2009 [10] similarly showed that Rintatolimod “led to more rapid decline of virus in the lungs compared with untreated animals” with a 100% survival outcome in the mouse study as opposed to a 100% death rate in the control group. These controlled studies evidence Rintatolimod's activity against SARS-CoV-1. The well-tolerated safety profile and bio-activity of Rintatolimod administered intranasally is well documented in both animal models [4,5,6] and humans [7].

### REFERENCES

[1] Gowen B B, Wong M-H, Jung K-H, Sanders A B, Mitchell W M, Alexopoulou L, et al. TLR3 is essential for the induction of protective immunity against Punta Toro virus infection by the double-stranded RNA (dsRNA), Poly(I:C12U), but not Poly(I:C): differential recognition of synthetic dsRNA molecules. J Immunol 2007; 178:5200-8.
[2] Hewson C A, Jardine A, Edwards M R, Laza-Stanca V, Johnston S L. Toll-like receptor 3 is induced by and mediates antiviral activity against rhinovirus infection of human bronchial epithelial cells. J Virol 2005; 79(19):12273-9.
[3] Uehara A, et al. Various human epithelial cells express functional Toll-like receptors, NOD1 and NOD2 to produce anti-microbial peptides, but not proinflammatory cytokines, Molecular Immunology 44 (2007) 3100-3111
[4] Ichinohe T, Kawaguchi A, Tamura S, Takahashi H, Sawa H, Ninomiya A, et al. Intranasal immunization with H5N1 vaccine plus Poly I: Poly C12U, a toll-like receptor agonist, protects mice against homologous and heterologous virus challenge. Microbes Infect 2007; 9:1333-40.
[5] Ichinohe T, Ainai A, Yasushi A, Nagata N, Iwata N, Kawaguchi A, et al. Intranasal administration of adjuvant-combined vaccine protects monkeys from challenge with the highly pathogenic influenza A H5N1 virus. J Med Virol 2010; 82:1754-61.
[6] Ichinohe T, Tamura S, Kawaguchi A, Imai M, Itamura S, Odagiri T, et al. Cross-protection against H5N1 influenza virus infection is afforded by intranasal inoculation with seasonal trivalent inactivated influenza vaccine. J Infect Dis 2007; 196:1313-20.
[7] Overton E T, et al. Intranasal seasonal influenza vaccine and a TLR-3 agonist, rintatolimod, increased cross-reactive IgA antibody formation against avian H5N1 and H7N9 influenza HA in humans. Vaccine 32 (2014) 5490-5495
[8] Strayer D R, Carter W A, Stouch B C, Stevens S R, Bateman L, Cimoch P J, et al. A double-blind, placebo-controlled, randomized, clinical trial of the TLR-3 agonist rintatolimod in severe cases of chronic fatigue syndrome. PLoS ONE 2012; 7(3):e31334.
[9] Barnard, D. L.; Day, C. W.; Bailey, K.; Heiner, M.; Montgomery, R.; Lauridsen, L.; Chan, P. K.; Sidwell, R W. Evaluation of immunomodulators, interferons and known in vitro SARS-CoV inhibitors for inhibition of SARS-CoV replication in BALB/c mice. Antivir Chem Chemother, 2006, 17(5), 275-84.
[10] Day, C. W.; Baric, R.; Cai, S. X.; Frieman, M.; Kumaki, Y.; Morrey, J. D.; Smee, D. F.; Barnard, D. L. A new mouse-adapted strain of SARS-CoV as a lethal model for evaluating antiviral agents in vitro and in vivo. Virology, 2009, 395(2), 210-222.

### Example 8: Universal Corona Virus Vaccine

Influenza continues to present a worldwide problem even with the existing vaccines with approximately 15-20 million cases annually. These yearly epidemics results in approximately 40,000 deaths in the United States alone.

More than 100 national influenza centers in over 100 countries conduct year-round surveillance for influenza. This involves receiving and testing thousands of influenza virus samples from patients and sending representative viruses to five World Health Organization (WHO) Collaborating Centers for Reference and Research on Influenza. Based on the submitted data, the local or regional authorities, such as the Food and Drug Administration (FDA) in the United States, then decide on the components of the next seasonal influenzas vaccine. Because it is not feasible to vaccinate a population against every flu strain discovered, only a subset of the flu strains are selected for inclusion in the vaccine. Because of this selection process, current influenza vaccines offer only limited protection against influenza strains that are not included in the vaccine.

Rintatolimod is a double-stranded RNA (dsRNA) and a generally well tolerated selective Toll-like receptor 3 (TLR3) agonist with induction of innate and adoptive immune responses. TLR3 is expressed in high concentrations on human airway epithelial cells and serves as a recognition system for many respiratory pathogens.

The mucosal surfaces of the nose and respiratory track serves as an ideal environment for Rintatolimod to exert its pronounced ability to enhance the innate response to respiratory pathogens like influenza virus, adenovirus, and coronavirus. Indeed, the intranasal instillation of inactivated or attenuated influenza viruses contained in seasonal influenza vaccine when used in combination with Rintatolimod has been shown to be able to induce a broad anti-viral IgA response with cross-reactivity against highly pathogenic human viruses such as various H5N1 clades (A/Indonesia 5/2000, A/Hong Kong/483/97, and A/Vietnam/1194/2001/) in mice, non-human primates, and humans.

The unique ability of Rintatolimod to be able to safely enhance the mucosal IgA response to both homologous as well as heterogenous strains of influenza virus is dependent on the interaction at the mucosal environment of Rintatolimod and foreign protein epitopes present in the viruses. This interaction results in epitope spreading and the generation of secretory IgA (S-IgA) with a very broad cross-reactivity against more distantly related clades and even different strains of viruses.

This immune enhancement process is not unique to the influenzas virus, but is also adaptable to other respiratory viruses such as coronavirus. Indeed, this disclosure utilized the recently isolated highly pathogenic coronavirus (SARS-CoV-2) isolated from patients with a severe respiratory infection that originated in Wuhan, China and has spread using human to human transmission around the world.

The methods of this disclosure include the use of a vaccine combined with Rintatolimod and administering intranasally (IN), with the generation of a mucosal S-IgA response and having a broad cross-reactivity against other coronavirus including SARS, MERS, and human coronaviruses 229E, NL63, and OC43. The vaccine may contain inactivated (dead) SARS-CoV-2, attenuated SARS-CoV-2, an antigen of SARS-CoV-2, an RNA encoding an antigen of SARS-CoV-2, or a similarly isolated virus from patients infected with SARS-CoV-2

This universal coronavirus vaccine would have antiviral activity not only against currently identified coronaviruses but also against newly emerging coronavirus that currently are in wild animal populations such as bats and are likely to emerge in the future to infect human populations similarly to SARS, MERS, and SARS-CoV-2.

### Example 9: Collecting and Detecting SARS-CoV-2 Virus

Nasopharyngeal and oropharyngeal swab specimens are collected with synthetic fiber swabs; each swab was inserted into a separate sterile tube containing 2 to 3 ml of viral transport medium using established techniques. See, e.g., Holshue et al., N Engl J Med 2020; 382:929-936.

Detection of SARS-CoV-2 may be made by polymerase chain reaction. Sequences for PCT are available through GenBank, for example, in accession number MN985325. Methods for detecting SARS-CoV-2, for example, by real-time reverse-transcriptase-polymerase-chain-reaction (rRT-PCR) assay, are known and published. See, e.g., Holshue et al., N Engl J Med 2020; 382:929-936.

### Example 10: Growing SARS-CoV-2 Virus In Vitro: Method 1

Methods for culturing cells that can host SARS-CoV-2 has been published in Journals. See, e.g., Harcourt et al., Emerging Infectious Diseases, Vol. 26, No. 6, June 2020, pages 1266-1273.

Human airway epithelial cell culture has been known for over 20 years (see, e.g., Lechner, J. F., Haugen, A., McLendon, I. A., and Pettis, E. W. (1982) Clonal growth of normal adult human bronchial epithelial cells in a serum-free medium. In Vitro 18, 633-642.). Human airway epithelial cells are harvested directly from humans according to established protocols (Jonsdottir H R, Dijkman R. Coronaviruses and the human airway: a universal system for virus-host interaction studies. Virol J 2016; 13:24-24). Human airway epithelial cell cultures maintained at an air-liquid interface (ALI) is known and well described (Fulcher, M. L., Gabriel, S.; Burns, K. A., Yankaskas, J. R., Randell, S. H., Well-Differentiated Human Airway Epithelial Cell Culture, in Methods in Molecular Medicine, Vol. 107: Human cell Culture Protocols, Second Edition, Edited by: J. Picot, Humana Press Inc. Totowa, N.J.). Primary cells such as “Normal Human Bronchial Epithelial Cells-P1” (catalog number: NhBE-P1) are also available commercially for example, by Novabiosis (North Carolina, U.S.A.). These cells are suitable for growing SARS-CoV-2 cells in vitro (Zhu, N., et al, “A Novel Coronavirus from Patients with Pneumonia in China, 2019”; published on the web from the New England Journal of Medicine Jan. 24, 2020).

Bronchoalveolar-lavage fluid are collect from infected subjects and the collected samples are centrifuged to remove cellular debris. The supernatants containing coronavirus (e.g., SARS-CoV-2) are propagated on human airway epithelial cells as described herein.

To prepare cells for virus propagation, human airway epithelial cells are expanded on plastic substrate to generate passage-1 cells and are subsequently plated at a density of 2.5×10⁵ cells per well on permeable Transwell-COL (12-mm diameter) supports. Human airway epithelial cell cultures are generated in an air-liquid interface for 4 to 6 weeks to form well-differentiated, polarized cultures resembling in vivo pseudostratified mucociliary epithelium.

Prior to infection, the apical surfaces of the human airway epithelial cells are washed three times with phosphate-buffered saline. Infection is initiated by adding 150 μl of supernatant from bronchoalveolar-lavage fluid samples (as described above) or from a previous SARS-CoV-2 preparation onto the apical surface of the cell cultures. After a 2-hour incubation at 37° C., unbound virus is removed by washing with 500 μl of phosphate-buffered saline for 10 minutes. The human airway epithelial cells are maintained in an air-liquid interface incubated at 37° C. with 5% carbon dioxide. Every 48 hours, 150 μl of phosphate-buffered saline is applied to the apical surfaces of the human airway epithelial cells, and after 10 minutes of incubation at 37° C. the samples are harvested as new SARS-CoV-2 virus harvests. The viral title may be monitored by RT-PCR and by infecting new cultures and observing cytopathic effects (CPE).

### Example 11: Example of Growing Host Cells Susceptible to SARS-CoV-2, Infecting the Cells with SARS-CoV-2, and Testing Rintatolimod

To test the efficacy of rintatolimod made by the methods of this disclosure, experiments were performed an in vitro study to determine the antiviral efficacy of rintatolimod against severe acute respiratory syndrome coronavirus 2 (SARS-CoV-2) in human-derived tracheal/bronchial epithelial cells. All rintatolimod in this Example were made by us following the procedures and methods of this disclosure.

The antiviral activity of AIM ImmunoTech's compound rintatolimod made according to the methods disclosed in this disclosure was evaluated against SARS-CoV-2 (strain USA-WA1/2020) in a highly differentiated, three-dimensional (3-D), in vitro model of normal, human-derived tracheal/bronchial epithelial (TBE) cells. The compounds were tested at 5 concentrations in triplicate inserts of the 3D tissue models of the human airway (MatTek Life Sciences) as indicated in Table 1. Antiviral activity was measured by virus yield reduction assays 5 days after infection.

Compounds: Rintatolimod was provided as 25 frozen 1 mL aliquots at 2.5 mg/mL and stored at −20° C. upon arrival. A fresh vial of the compound was diluted to the test dilutions (100, 50, 25, 12.5, and 6.25 μg/mL) in the MatTek culture medium (AIR-100-MM) just prior to each drug addition using RNase-free tubes, pipettes and pipet tips. Remdesivir (MedChemExpress, cat #HY-104077) was tested in singlet wells at 1, 0.1, 0.01, and 0.001 μg/mL as the positive control.

Cell Culture: The EpiAirway™ Model consists of normal, human-derived tracheal/bronchial epithelial (TBE) cells which have been cultured to form a multi layered, highly differentiated model which closely resembles the epithelial tissue of the respiratory tract. The cell cultures were made to order by MatTek Life Sciences (https://www.mattek.com) (Ashland, Mass.) and arrived in kits with either 12- or 24-well inserts each. The TBE cells were grown on 6 mm mesh disks in transwell inserts. During transportation the tissues were stabilized on a sheet of agarose, which was removed upon receipt. One insert was estimated to consist of approximately 1.2×10⁶ cells. Kits of cell inserts (EpiAirway™ AIR-100) originated from a single donor, #9831, a 23-year old, healthy, non-smoking, Caucasian male. The cells have unique properties in forming layers, the apical side of which is exposed only to air and that creates a mucin layer. Upon arrival (Tuesday, 4 Aug. 2020), the cell transwell inserts were immediately transferred to individual wells of a 6-well plate according to manufacturer's instructions, and 1 mL of MatTek's proprietary culture medium (AIR-100-MM) was added to the basolateral side, whereas the apical side was exposed to a humidified 5% CO₂ environment. The TBE cells were cultured at 37° C. for 5 h.

Viruses: SARS-CoV-2 strain USA-WA1/2020 was passaged three times in Vero 76 cells to create the virus stock. Virus was diluted in AIR-100-MM medium before infection, yielding a multiplicity of infection (MOI) of approximately 0.005 CCID₅₀ per cell.

Experimental design: After the 5 h equilibration period, the cells were treated with drug on the basal side of the transwells and cultured at 37° C. for 18 h. The mucin layer, secreted from the apical side of the cells, was removed by washing with 400 μL pre-warmed 30 mM HEPES buffered saline solution 3×. Each compound treatment (120 μL) and virus (120 μL) was applied to the apical side, and compound treatment only was applied to the basal side (1 mL), for a 2 h incubation. As a virus control, 4 of the cell wells were treated with placebo (cell culture medium only). Following the 2 h infection, the apical medium was removed, and the basal side was replaced with fresh compound or medium. The cells were maintained at the air-liquid interface. The basal side compound was replaced again at 6 h, 36 h, 48 h, and 72 h after the infection. On day 5, the medium was removed and discarded from the basal side. Virus released into the apical compartment of the tissues was harvested by the addition of 400 μL of culture medium that was pre-warmed at 37° C. The contents were incubated for 30 min, mixed well, collected, thoroughly vortexed and plated on Vero 76 cells for VYR titration. Triplicate and singlet wells were used for virus control and cell controls, respectively.

Determination of virus titers from each treated cell culture: Vero 76 cells were seeded in 96-well plates and grown overnight (37° C.) to 90% confluence. Samples containing virus were diluted in 10-fold increments in infection medium and 200 μL of each dilution transferred into respective wells of a 96-well microtiter plate. Four microwells were used for each dilution to determine 50% viral endpoints. After 5 days of incubation, each well was scored positive for virus if any cytopathic effect (CPE) was observed as compared with the uninfected control, and counts were confirmed for endpoint on day 7. The virus dose that was able to infect 50% of the cell cultures (CCID₅₀ per 0.1 mL) was calculated by the Reed-Muench method (1948). The day 5 values are reported. Untreated, uninfected cells were used as the cell controls.

MTT cytotoxicity assay: The MTT assay is used as an indicator of cell viability. The colorimetric assay is based on the reduction of a yellow tetrazolium salt to purple formazan crystals by live cells. The formazan crystals are then dissolved using a solubilization solution (10% SDS prepared in PBS) and the resulting colored solution is quantified by measuring absorbance at 550 nanometers using a multi-well spectrophotometer.

The test compound was provided as 3 frozen 6 mL aliquots at 10.3 mg/mL prepared in MatTek's proprietary culture medium (AIR-100-MM and stored at −20° C. upon arrival. A fresh vial of the compound was diluted to the test dilutions (10, 4.5, 1.5, and 0.5 mg/mL) in the MatTek culture medium just prior to each drug addition using RNase-free tubes, pipettes and pipet tips. Two tissues treated with medium only were used as the cell controls.

After the 24 h equilibration period, the mucin layer, secreted from the apical side of the cells, was removed by washing with 400 μL pre-warmed 30 mM HEPES buffered saline solution 3×. Each compound treatment (240 μL) was applied to the apical side and to the basal side (1 mL), for a 2 h incubation at 37° C. As a cell control, 2 cell wells were treated with placebo (cell culture medium only). Following the 2 h incubation (to mimic the conditions of a virus infection), the apical medium was removed, and the cells were maintained at the air-liquid interface. The basal side compound was replaced again at 48 and 96 h after the mock infection. On day 5, the apical side was washed 1× with PBS, 0.1 mL of MTT was applied to the apical side and incubated at 37° C. overnight for the cytotoxicity assay. Any remaining liquid was then removed from the apical and basal sides and 0.2 mL of solubilization solution added to the cell inserts and incubated at 37° C. overnight. The solution was then transferred to a 96-well flat-bottom plate and read by a spectrophotometer. Triplicate and singlet wells were used for virus control and cell controls, respectively.

### Results

The virus yield results and EC90 values are summarized in Table 1.

Rintatolimod tested at 10 mg/mL was 47% cytotoxic, 4.5 mg/mL was 12% cytotoxic, and the lower concentrations had no measurable toxicity. The data indicate that the cell cytotoxicity concentration of compound that would cause 50% cell death (CC₅₀) is >10 mg/mL in the tested tissue model of normal, human-derived tracheal/bronchial epithelial cells.

Reference: Reed, L. J., Muench, H., 1938. A simple method of estimating fifty percent endpoints. The American Journal of Hygiene 27, 493-497.

TABLE 6
Antiviral efficacy: EC90 for AIM ImmunoTech, Inc. compound
rintatolimod against SARS-CoV-2.

| | Concentration | ^{a}Log10 CCID50 | ^{b}EC90 |
|---|---|---|---|
| Test Compounds | (μg/mL) | virus per 0.2 mL | (μg/mL) |
| Rintatolimod | 100 | 3.00 | 49 |
| | 50 | 3.67 | |
| | 25 | 4.50 | |
| | 12.5 | 4.67 | |
| | 6.25 | 4.50 | |
| Rintatolimod | 100 | 3.00 | 55 |
| | 50 | 4.00 | |
| | 25 | 4.30 | |
| | 12.5 | 4.30 | |
| | 6.25 | 4.50 | |
| Rintatolimod | 100 | 3.50 | 39.1 |
| | 50 | 3.50 | |
| | 25 | 4.00 | |
| | 12.5 | 4.30 | |
| | 6.25 | 4.30 | |
| Remdesivir | 1 | 3.00 | 0.01 |
| | 0.1 | 3.00 | |
| | 0.01 | 3.67 | |
| | 0.001 | 4.30 | |
| Virus Control | | 5.00 | |
| | | 4.00 | |
| | | 4.67 | |
| | | 5.00 | |

Each well was scored positive for virus if any CPE was observed as compared with the uninfected control. Vero 76 cells were scored on day 5 and confirmed on day 7.
^{a}Titer results from the virus yield reduction (VYR) assay.
^{b}EC90 = 90% effective concentration (concentration to reduce virus yield by 1 log10) determined by regression analysis.

Our results indicate that the SARS-CoV-2 virus count can be reduced by one order of magnitude, to 10 fold less, when tdsRNA is applied at a concentration of 55 μg/mL. To confirm that this is a safe dosage for application to humans in a clinical setting, this concentration was compared to clinically achievable concentration based on the intranasal safety profile of rintatolimod as shown below.

TABLE 7
The EC₉₀ of Rintatolimod Against SARS-CoV-2 in a 3-D In Vitro Model
of Normal, Human-derived Tracheal/Bronchial Epithelial Cells was 39.1-
55 μg/ml, a Clinically Achievable Concentration Based on the Intranaal
Safety Profile of Rintatolimod.

| Intranasal | Dose | Dose | Rintatolimod | Fold Dose Conc. |
|---|---|---|---|---|
| Rintatolimod | Volume¹ | Concentration | EC₉₀² | Increase Over |
| dose (μg) | (μl) | (μg/ml) | (μg/ml) | EC₉₀ |
| 200 | 500 | 400 | 55 | 7.3 |
| 500 | 500 | 1000 | 55 | 18.2 |
| 1250 | 500 | 2500 | 55 | 45.5 |

¹Dose volume is split equally between each nostril.
²Highest EC90 value obtained.
For the well tolerated dose concentration of 2500 μg/ml, it represents a 45.5 fold increase relative to the EC90 at 55 μg/ml.

As can be seen, rintatolimod made by the methods of this disclosure has high antiviral activity against SARS-CoV-2 as shown by EC₉₀ at rintatolimod concentrations that are well tolerated in humans. In fact, as shown in Table 7 above, a dosage that is 45 fold higher (i.e, 45×EC₉₀ dose) is well tolerated in humans. Thus, tdsRNA and especially tdsRNA made by the process of this disclosure is effective and well tolerated for the treatment or prevention of SARS-CoV-2 infection or for the reduction of SARS-CoV-2 titer on nasal tissue.

Rintatolimod has been tested in vitro in a SARS-CoV-2 infection model in human-derived tracheal/bronchial epithelial cells. Rintatolimod decreased SARS-CoV-2 infectious viral yields by 90% (EC90) at clinically achievable intranasal dosage levels (Table 6). In the same human-derived tracheal/bronchial epithelial cell system, the cell cytotoxicity 50% (CC50) of Rintatolimod was >10 mg/mL. Rintatolimod concentrations of 1.5 mg/ml and 0.5 mg/ml induced no cellular toxicity (0%).

As a further test of the ability of tdsRNA to enhance protection against SARS-CoV-2, the following experiments were performed as on animal hosts as follows:

“0 day” and “0 Week” is defined as the day and week of SARS-CoV-2 infection. Therefore, −1 week refers to 1 week before infection or −7 days before infection, 10 days refers to 10 days after infection. Blood sampling was performed throughout the period of experiments.

Mice were immunized at −35 days and −21 days. The dosage of SARS-CoV-2 S protein ectodomain (referred to in this Example only as S protein) is at 100 ng per mouse when administered. The dosage of tdsRNA, in the form of rintatolimod (rIₙ.r(C₁₂U)ₙ), was at 10 μg per mouse when administered. All immunizations were performed by subcutaneous injection.

Group 1 mice were administered S protein only. Group 2 mice were administered S protein and tdsRNA. Group 3 mice were sham administered phosphate buffered saline. As discussed, infection was at 0 day.

Neutralization antibody in the serum was measured at −7 days which is 2 weeks after the second immunization but before infection. Group 3 mice (sham immunized) has a relative titer of 2 (log 2) representing a baseline of the measuring methods. Group 1 mice (S protein only) has a relative titer of 4 (log 2). Group 2 mice (S protein and tdsRNA) has a relative titer of 16 (log 2).

Viral titers after infection were measured. Group 3 mice (sham immunized) has a viral titer (Log 10/g) of 8.7. Group 1 mice (S protein only) has a viral titer (Log 10/g) of 8. Group 2 mice (S protein and tdsRNA) have a viral titer (Log 10/g) of 6, 1/10 of the Group 1 mice because the scale is Log₁₀/g.

Group 3 mice (sham immunized) lost weight linearly until they reached 70% of their initial weight 5 days after infection at which point they died from the infection. Group 1 mice (S protein only) had a weight reduction to 83% by day 3 and about 95% by day 10. Group 2 mice (S protein and tdsRNA) had a weight reduction to 87% by day 3 and gained weight by day 10 to reach a level of 105%. All weight percent were measured as a percentage of initial weight at the moment of infection which was set as 100%.

Survival data were most dramatic. Group 3 mice (sham immunized) 1/3 of the mice died at day 5 and all mice died by day 6. Group 1 mice (S protein only) had 1/9 death by day 6 and survival was 8/9 by day 10. In contrast, Group 2 mice (S protein and tdsRNA) had 100% survival by day 10.

### Example 12: Growing SARS-CoV-2 Virus In Vitro: Method 2

SARS-CoV has been shown to replicate in BGM, CV-1, FRhK, LLC-Mk2, MA-104, pCMK, RK-13, and Vero cell lines. These cell lines produced a cytopathic effect (CPE) (also termed cell death) as early as day 4 after inoculation (Kaye, M., Druce, J., Tran, T., Kostecki, R., Chibo, D., Morris, J., Catton, M., and Birch, C. Emerg Infect Dis. 2006 January; 12(1): 128-133.).

Vero E6 cells are grown in Vero E6 cell growth media which is formulated as follows: minimal essential media (MEM) supplemented with 10% heat inactivated fetal calf serum (FCS), 1% L-glutamine and 1% penicillin/streptomycin. Vero E6 cells may be grown in T flasks such as, for example, NUNC T Flask (e.g., from suppliers such as ThermoFisher Scientific) in T25, T75, T175, and T225 sizes. Passage and growth may be performed using standard tissue culture techniques.

For SARS-CoV-2 virus growth, seed 1×10⁷ Vero E6 cells into a T175 flask and culture at 37° C. in 5% CO₂ to achieve a 90% confluent layer. Remove growth media and wash cells with serum free media leaving about 5 ml serum free media in a T175 flask. Add 1 ml of SARS-CoV-2 virus to the flask. Distribute virus over cells and incubate for 1 hour at 37° C. in 5% CO₂. Replenish with growth media up to a total volume of 20 ml in a T175 flask. Incubate flask for 48-72 hours at 37° C. in 5% CO₂, or until significant cytopathic effect (CPE) (also termed cell death) is observed. Collect supernatant from the infected flask and centrifuge at 500 g for 5 minutes to remove any cellular debris. Aliquot appropriate volumes (100 μl-1 ml) of the clarified supernatant into 1.5 ml screw cap tubes and store at −80° C. until needed.

The same procedure scaled down can be used to titer a sample of SARS-CoV-2 virus. For example, Vero E6 cells can be grown in 96 well plates with each well seeded with 1×10⁴ Vero E6 cells. Each well can be infected with serial dilutions of virus and cytopathic effect (CPE) (also termed cell death) for each well is observed. The titer of the SARS-CoV-2 preparation is then determined by statistical methods. Alternatively, SARS-CoV-2 titer may be determined by a plaque assay.

The produced virus may be inactivated using standard industrial techniques such as formaldehyde inactivation (24 hr at 2-7° C.) is performed at a final concentration of 0.02% formalin. Another standard inactivation technique is beta-propiolactone (BPL) based inactivation (24 hr at 18-22° C.) with a final BPL concentration of 0.1%.

### Example 13: Growing SARS-CoV-2 Virus In Vitro: Method 3

Vero CCL-81 cells can be used for in vitro growth and amplification, isolation, and initial passage of SARS-CoV-2. Vero E6, Vero CCL-81, HUH 7.0, 293T, A549, and EFKB3 cells in Dulbecco minimal essential medium (DMEM) supplemented with heat-inactivated fetal bovine serum (5% or 10%) and antibiotics/antimycotics (GIBCO, https://www.thermofisher.com).

Virus isolation is started using both nasopharyngeal (NP) and oropharyngeal (OP) swabs. For isolation, limiting dilution, and passage 1 of the virus the following procedures may be used. 50 μl of serum-free DMEM is added to columns 2-12 of a 96-well tissue culture plate, then 100 μl of clinical specimens are pipetted into column 1 and serially diluted 2-fold across the plate. Vero cells are trypsinized and resuspended in DMEM containing 10% fetal bovine serum, 2× penicillin/streptomycin, 2× antibiotics/antimycotics, and 2× amphotericin B at a concentration of 2.5×10⁵ cells/ml. 100 μl of cell suspension is added directly to the clinical specimen dilutions and mixed gently by pipetting. The inoculated cultures are grown in a humidified 37° C. incubator in an atmosphere of 5% CO2 and observed for cytopathic effects (CPEs) daily. Standard plaque assays for SARS-CoV-2 can be used to monitor virus growth. This protocol is based on SARS-CoV and Middle East respiratory syndrome coronavirus (MERS-CoV) which are known and published.

When CPEs are observed, cell monolayers are scraped with the back of a pipette tip. 50 μL of viral lysate are used for total nucleic acid extraction for confirmatory testing and sequencing. 50 μL of virus lysate are used to inoculate a well of a 90% confluent 24-well plate.

Confirmatory testing, to determine that we are growing SARS-CoV-2 are performed by using real-time reverse transcription PCR (CDC) and full-genome sequencing. Cells in which CPE is observed are used for testing and confirmation. The CDC molecular diagnostic assay targets 3 portions of the nucleocapsid gene, and results for all 3 portions should be positive for a sample to be considered positive (https://www.cdc.gov/coronavirus/2019-ncov/lab/rt-per-detection-instructions.html and https://www.cdc.gov/coronavirus/2019-ncov/lab/rt-per-panel-primer-probes.html). To, Fast Track Respiratory Pathogens 33 Testing (FTD Diagnostics,) http://www.fast-trackdiagnostics.com) is used to confirm that no other respiratory viruses were present.

### Example 14: Generating Anti-SARS-CoV-2 Antibodies

Methods for generating SARS-CoV-2 antibodies are published in Journals. See, e.g., Harcourt et al., Emerging Infectious Diseases, Vol. 26, No. 6, June 2020, pages 1266-1273.

Plasmid pBM302 (Das D, Suresh M R. Copious production of SARS-CoV spike protein employing codon optimized synthetic gene is used to express a SARS-CoV-2 nucleocapsid protein, with a C-terminal His6 tag, to high levels within the inclusion bodies of Escherichia coli. The recombinant protein is purified from the inclusion bodies by using nickel-affinity column chromatography under denaturing conditions. Stepwise dialysis against Tris/phosphate buffer is performed to refold the recombinant SARS-CoV-2 nucleocapsid protein with decreasing concentrations of urea to renature the protein. Rabbits are immunized with the renatured, full-length, SARS-CoV-2 nucleocapsid protein to generate an affinity-purified rabbit anti-SARS-CoV-2 nucleocapsid protein polyclonal antibody.

### Example 15: Nasal Immunization Using SARS-CoV-2, tdsRNA, and a Combination Thereof

SARS-CoV-2 virus is grown in vitro using published techniques as discussed in this disclosure or using protocols as disclosed in the Examples section. The collected virus supernatant from cell cultures is purified at 1500×g for 20 min to remove cell debris. For inactivation, the purified virus is treated with 1:4000 (v/v) formalin and incubated for 3 days at 37° C. and then dialyzed against PBS. Inactivation of virus is confirmed by inoculation of the virus into cells permissible for virus growth as disclosed in the other Examples—preferably Example 5.

Antigen:

Three types of nasal administration composition are prepared with 3 types of antigens.

The first type comprises inactivated virus only. For nasal immunization, 50, 100, 200, 500, or 1 mg of inactivated virus may be used. In a preferred embodiment, the amount of inactivated viruses may be 5 μg to 10 μg; 10 μg to 20 μg; 20 μg to 50 μg; 50 μg to 100 μg; 100 μg to 200 μg; 200 μg to 500 μg; 500 μg to 1000 μg; 1000 μg to 1500 μg; 1500 μg to 2000 μg; or any combination thereof.

The second type comprises inactivated virus and tdsRNA. As stated above, for nasal immunization, 50, 100, 200, 500, or 1 mg of inactivated virus is used. In a preferred embodiment, the amount of inactivated viruses may be 5 μg to 10 μg; 10 μg to 20 μg; 20 μg to 50 μg; 50 μg to 100 μg; 100 μg to 200 μg; 200 μg to 500 μg; 500 μg to 1000 μg; 1000 μg to 1500 μg; 1500 μg to 2000 μg; or any combination thereof. In addition, about an equal weight amount of tdsRNA is also added. That is, any of the above listed dosage for inactivated virus may also apply to the tdsRNA.

The third type comprises tdsRNA without any inactivated virus. 50,100,200, 500, or 1 mg of tdsRNA may be used. In a preferred embodiment, the amount of tdsRNA may be 5 μg to 10 μg; 10 μg to 20 μg; 20 μg to 50 μg; 50 μg to 100 μg; 100 μg to 200 μg; 200 μg to 500 μg; 500 μg to 1000 μg; 1000 μg to 1500 μg; 1500 μg to 2000 μg; or any combination thereof.

For any nasal administration composition, including type 1, type 2 and type 3 listed above, the composition may optionally include Cholera Toxin. Cholera toxin B Subunit is optionally added in equal weight amounts (e.g., 50, 100, 200, 500, or 1 mg) for its synergistic effect to stimulate nasal immunity. Cholera toxin (CT) may be purchased from Sigma-Aldrich (St. Louis, Mo.). Therefore, optionally, 6 different types (3 types without cholera toxin, and 3 types with cholera toxin are prepared.

Immunization

Humans and animals, such as ferrets, susceptible to SARS-CoV-2 are used for intranasal immunizations. Nasal immunization may comprise the above listed dosages for nasal immunization in PBS in a total volume of between 25 μL to 100 μL.

To determine the titers of SARS-CoV-2 in the lungs of infected naive and immunized animals (such as ferrets and not humans) will be challenged by intranasal instillation of 25 μL of SARS-CoV-2 (e.g., about 600 to 6000 pfu) 11 weeks after the last immunization. The challenged animals is monitored for signs of morbidity (body weight changes, fever and hunched posture) and mortality. Animals are weighed immediately before and daily after challenge. Half of the animals from each group will be analyzed on day 4 and day 8 post-challenge. Lung homogenates will be prepared in DMEM serum-free medium to assess the viral titers, determined per g of lung tissue. See, e.g., Sha et al., Induction of CD4(+) T-cell-independent immunoglobulin responses by inactivated influenza virus. J Virol. 2000; 74(11):4999-5005. For plaque assays, we prepare serial dilutions of lung supernatants, incubate them with cells permissive for growth of SARS-CoV-2 as shown in the Examples.

Evaluation of Humoral Immune Responses The concentrations of virus-specific IgG, IgG1, IgG2a and IgA will be determined in all sera and mucosal secretions using standard assay procedures such as ELISA plates coated with purified inactivated SARS-CoV-2. See, e.g., Sha et al., Induction of CD4(+) T-cell-independent immunoglobulin responses by inactivated influenza virus. J Virol. 2000; 74(11):4999-5005. Kang et al. Enhancement of mucosal immunization with virus-like particles of simian immunodeficiency virus. J Virol. 2003; 77(6):3615-23. Kang et al. Intranasal immunization with inactivated influenza virus enhances immune responses to coadministered simian-human immunodeficiency virus-like particle antigens. J Virol. 2004; 78(18):9624-32.

We determine the hemagglutination inhibition (HI) and neutralizing antibody titers, which are both used as indicators of protective immune responses to viruses, as previously described. Sha et al., Induction of CD4(+) T-cell-independent immunoglobulin responses by inactivated influenza virus. J Virol. 2000; 74(11):4999-5005. Novak et al., Murine model for evaluation of protective immunity to influenza virus. Vaccine 1993; 11(1):55-60.

Cellular Immune Responses is determined by Cytokine ELISA. Briefly, spleen or inguinal lymph nodes cells will be prepared from immunized mice at 2 weeks after the last immunization, and stimulated in vitro with inactivated SARS-CoV-2 virus at a final concentration of 1 μg/ml in complete RPMI medium. Sha et al., Induction of CD4(+) T-cell-independent immunoglobulin responses by inactivated influenza virus. J Virol. 2000; 74(11):4999-5005. Kang et al. Enhancement of mucosal immunization with virus-like particles of simian immunodeficiency virus. J. Virol. 2003; 77(6):3615-23. Kang et al. Intranasal immunization with inactivated influenza virus enhances immune responses to coadministered simian-human immunodeficiency virus-like particle antigens. J. Virol. 2004; 78(18):9624-32. After 72 h the cells are centrifuged and the supernatant will be collected and stored at −80° C. until assayed. Cytokine production (TNF-alpha, IFN-gamma, IL-4, IL-6 and IL-10) will be determined according to the manufacturer's instructions.

1. A composition for treating or preventing a viral infection caused by a virus in a subject, wherein the composition comprises a therapeutic double-stranded RNA (tdsRNA), wherein the tdsRNA is at least one selected from the group consisting of
rIₙ.r(CₓU)ₙ (formula 1);
rIₙ.r(CₓG)ₙ (formula 2);
rAₙ.rUₙ (formula 3);
rIₙ.rCₙ (formula 4); and
rugged dsRNA (formula 5);
wherein x is one or more at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

2. The composition of claim 1, wherein the composition further comprises a vaccine against the virus.

3. The composition of claim 1, wherein the virus is a virus of Table 2.

4. The composition of claim 1, wherein the virus is a coronavirus, preferably a SARS CoV 2 virus.

5. The composition of claim 1, wherein the virus is at least one selected from the group consisting of Human coronavirus 229E (HCoV-229E); Human coronavirus NL63 (HCoV-NL63, New Haven coronavirus); Human coronavirus OC43 (HCoV-OC43); Human coronavirus HKU1; Middle East respiratory syndrome-related coronavirus (MERS-CoV); novel coronavirus 2012 (HCoV-EMC); Severe acute respiratory syndrome-related coronavirus (SARS-CoV); Severe acute respiratory syndrome coronavirus 2 (SARS-CoV-2); Ebola Virus; H5 influenza; H7 influenza; H5N1 influenza; Influenza A; Influenza B; H1N1 influenza; H3N2 influenza; H7N9 influenza; H5N6 influenza; H10N8 influenza; H9N2 influenza; H6N1 influenza; West Niles Virus; and Zika Virus.

6. The composition of claim 1, wherein n is a number with a value of 40 to 50,000.

7. The composition of claim 1, wherein at least 90 wt % of the tdsRNA is larger than 40 basepairs.

8. The composition of claim 1, wherein at least 90 wt % of the tdsRNA is smaller than 50,000 basepairs.

9. The composition of claim 1,
wherein n is from 40 to 40,000;
wherein the tdsRNA has about 4 to about 4000 helical turns of duplexed RNA strands; or
wherein the tdsRNA has a molecular weight of
2 kDa to 30,000 kDa.

10. The composition of claim 1, wherein the tdsRNA comprises
rIₙ.ribo(C₁₁₋₁₄U)ₙ; and
rugged dsRNA.

11. The composition of claim 1, wherein the rugged dsRNA has
a single strand comprised of r(C₄₋₂₉U)ₙ, r(C₁₁₋₁₄U)ₙ, or r(C₁₂U)ₙ; and
an opposite strand comprised of r(I);
wherein the single strand and the opposite strand do not base pair the position of the uracil base, and
wherein the single strand and the opposite strand are partially hybridized.

12. The composition of claim 1, wherein
the rugged dsRNA has a molecular weight of about 250 kDa to 500 kDa;
each strand of the rugged dsRNA is from about 400 to 800 basepairs in length; or
the rugged tdsRNA has about 30 to 100 or 30-60 helical turns of duplexed RNA.

13. The composition of claim 1, wherein the tdsRNA is Rugged dsRNA which is resistant to denaturation under conditions that are able to separate hybridized poly(riboinosinic acid) and poly(ribocytosinic acid) strands (rIₙ.rCn).

14. The composition of claim 1, wherein the rugged dsRNA is an isolated double-stranded ribonucleic acid (dsRNA) enzymatically active under thermal stress comprising:
each strand with a molecular weight of about 250 KDa to about 500 KDa, 400-800 basepairs, or 30 to 60 helical turns of duplex RNA,
a single strand comprised of poly(ribocytosinic₄₋₂₉ uracilic acid) and an opposite strand comprised of poly(riboinosinic acid),
wherein the two strands do not base pair the position of the uracil base,
wherein the two strands base pair the position of the cytosine base, and
wherein said strands are partially hybridized.

15. The composition of claim 1, wherein the tdsRNA is produced by a method that comprises:
a) synthesizing a first single-stranded RNA (first ssRNA) in a first synthesis reaction with PNPase as the only RNA polymerase, and purifying said first ssRNA after the first synthesis reaction;
b) synthesizing a second single-stranded RNA (second ssRNA) in a second synthesis reaction with PNPase as the only RNA polymerase, and purifying said second ssRNA after the second synthesis reaction; and
c) hybridizing the first ssRNA with the second ssRNA to form the tdsRNA;
wherein step a) and step b) are performed in any order;
wherein the first synthesis reaction comprises inosine diphosphate (rIDP) as the only free ribonucleotide;
wherein the second synthesis reaction comprises cytidine diphosphate (rCDP) and uridine diphosphate (rUDP) as the only two free ribonucleotides and a molar ratio of (free rCDP):(free rUDP) in the second synthesis reaction is about (11 to 14):(1).

16. The composition of claim 1, wherein the tdsRNA comprises 0.1-12 mol % rugged dsRNA.

17. The composition of claim 1, wherein the composition comprises at least one pharmaceutically acceptable carrier.

18. The composition of claim 1, wherein the tdsRNA is complexed with a stabilizing polymer.

19. The composition of claim 18, wherein the stabilizing polymer is at least one selected from the group consisting of:
polylysine; polylysine and carboxymethylcellulose; polyarginine; polyarginine and carboxymethylcellulose; and a combination thereof.

20. The composition of claim 1, wherein the composition further comprises an antiviral agent which is not a tdsRNA.

21. The composition of claim 20, wherein the antiviral agent is at least one selected from the group consisting of:
an antibody to an S protein of SARS-CoV-2; an antibody to a NTD region of a S protein of SARS-CoV-2; an antibody to a HR1 region of a S protein of SARS-CoV-2; an antibody to a RBD region of a S protein of SARS-CoV-2; a SARS-CoV monoclonal antibody; a MERS-CoV monoclonal antibody; a SARS-CoV-2 monoclonal antibody; a peptide; a protease inhibitor; a PIKfyve inhibitor; a TMPRSS2 inhibitor; a cathepsin inhibitor; a furin inhibitor; an antiviral peptide; an antiviral protein; an antiviral chemical compound; and an antiviral agent.

22. The composition of claim 20, wherein the antiviral agent is at least one selected from the group consisting of:
1A9; 201; 311mab-31B5; 311mab-32D4; 47D11; 4A8; 4C2; 80R; Apilimod; B38; camostat mesylate; Casirivimab; CR3014; CR3022; D12; E-64D; EK1; EK1C4; H4; HR2P; IBP02; Imdevimab; m336; MERS-27; MERS-4; MI-701; n3088; n3130; P2B-2F6; P2C-1F11; PI8; S230; S309; SARS-CoV-2 S HR2P fragment (aa1168-1203); Tetrandrine; Viracept (nelfinavir mesylate); YM201636; α-1-PDX; favipiravir; IFN-α; IFN-α1b; IFN-α2a; lopinavir-ritonavir; Q-Griffithsin (Q-GRFT); Griffithsin; oseltamivir; zanamivir; abacavir; zidovudine; zalcitabine; didanosine; stavudine; efavirenz; indinavir; ritonavir; nelfinavir; amprenavir; ribavirin; Remdesivir; chloroquine; hydroxychloroquine; rIFN-alpha-2a; rIFN-beta-1b; rIFN-gamma; nIFN-alpha; nIFN-beta; nIFN-gamma; IL-2; PD-L1; Anti-PD-L1; a checkpoint inhibitor; an interferon; interferon mixture; recombinant or natural interferon; Alferon; alpha-interferon species; recombinant or natural interferon alpha; recombinant or natural interferon alpha 2a; recombinant or natural interferon beta; recombinant or natural interferon beta 1b; and recombinant or natural interferon gamma.

23. The composition of claim 22, wherein the alpha-interferon species is a mixture of at least seven species of alpha-interferon produced by human white blood cells, wherein the seven species are: interferon alpha 2; interferon alpha 4; interferon alpha 7; interferon alpha 8; interferon alpha 10; interferon alpha 16; and interferon alpha 17.

24. The composition of claim 1, wherein the composition is at least one selected from the group consisting of an aqueous solution, a powder, a dry particle, a liquid particle, a gel particle, a semidry particle, an isotonic formulation, and a composition for nasal administration.

25. The composition of claim 2, wherein the vaccine comprises at least one selected from the group consisting of: an inactivated virus, an attenuated virus, a virus antigen, and a messenger RNA encoding protein comprising a virus antigen.

26. The composition of claim 25, wherein the virus antigen is an antigen from the S, E, M, or N structural protein of SARS-CoV-2.

27. A method for treating a viral infection caused by a virus in a subject comprising:
determining that the subject is infected by the virus; and
administering an effective amount of a composition of claim 1, to the subject infected by the virus.

28. The method of claim 27, wherein the subject has been infected by the virus for not more than two to seven days, or up to 14 days.

29. A method for preventing a viral infection caused by a virus in a subject comprising:
determining that the subject is not infected by the virus; and
administering an effective amount of a composition of claim 1, to the subject not infected by the virus.

30. A method for treating a viral infection caused by a virus in a subject:
administering a composition comprising an effective amount of a composition of claim 1, to the subject who has been infected with the virus,
is at risk for being infected by the virus because of exposure a second subject infected with the virus, or
is at risk for being infected by the virus because of presence in an area where there are reported cases of virus infection.

31. A method for immunizing a subject against a viral infection caused by a virus, the method comprising:
administering to the subject at least a first compound and a second compound in any order together or separately,
wherein the first compound comprises an effective amount of a vaccine, and
wherein the second compound is an effective amount of a composition of claim 1.

32. The method of claim 31, wherein the vaccine comprises at least one selected from the group consisting of: an inactivated virus, an attenuated virus, a virus antigen, and a messenger RNA encoding a virus antigen.

33. The method of claim 31, wherein the method produces an immune response in the subject.

34. The method of claim 33, wherein the immune response is at least one selected from the group consisting of virus-specific immunoglobulin production; virus specific IgG production; virus specific IgG1 production; virus specific IgG2a production; virus specific IgA production; and virus specific IgM production.

35. The method of claim 31, wherein the virus antigen is an antigen from the S, E, M, or N structural protein of SARS-CoV-2.

36. The method of claim 31, wherein the method induces an increased cross-reactive immune response and cross protection against a second different virus in a subject.

37. The method of claim 34, wherein the second different virus is a variant, a different strain, or a mutation of the virus.

38. The method of claim 31, wherein the method provides a vaccine effect that is superior than a coronavirus antigen administered alone.

39. The method of claim 31,
wherein the first compound and second compound is administered together as a mixture; or
wherein the first compound and second compound is administered at the same time or separately.

40. The method of claim 31, wherein the first compound and second compound is administered separately but within a time period selected from the group consisting of: 2 months; 1 month; 3 weeks; 2 weeks; 1 week; 3 days; 1 day; 12 hours, 6 hours, 3 hours, 2 hours, 1 hour, and 30 minutes.

41. The method of claim 27, wherein the virus or second different virus is a virus of Table 2.

42. The method of claim 27, wherein the virus is a coronavirus, preferably a SARS CoV 2 virus.

43. The method of claim 27, wherein the virus is at least one virus selected from the group consisting of Human coronavirus 229E (HCoV-229E); Human coronavirus NL63 (HCoV-NL63, New Haven coronavirus); Human coronavirus OC43 (HCoV-OC43); Human coronavirus HKU1; Middle East respiratory syndrome-related coronavirus (MERS-CoV); novel coronavirus 2012 (HCoV-EMC); Severe acute respiratory syndrome-related coronavirus (SARS-CoV); Severe acute respiratory syndrome coronavirus 2 (SARS-CoV-2); Ebola Virus; H5 influenza; H7 influenza; H5N1 influenza; Influenza A; Influenza B; H1N1 influenza; H3N2 influenza; H7N9 influenza; H5N6 influenza; H10N8 influenza; H9N2 influenza; H6N1 influenza; West Niles Virus; and Zika Virus.

44. The method of claim 27, wherein the effective amount is a therapeutically effective amount or a prophylactically effective amount of the tdsRNA.

45. The method of claim 27, wherein administering is at least one administering method selected from the group consisting of: intravenous administration; intradermal administration; subcutaneous administration; intramuscular administration; intranasal administration (pulmonary airway administration); intranasal administration and oral administration; intraperitoneal administration; intracranial administration; intravesical administration; oral administration (through the mouth, by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration; and a combination thereof.

46. The method of claim 45, wherein intranasal administration is at least one selected from the group consisting of: administering to nasal passages; administering to nasal epithelium; administering to lung; administering by inhalation; administering to the larynx; administering to bronchi; administering to alveoli; administering by inhalation; administering by nasal instillation; and a combination thereof.

47. The method of claim 27, wherein administering is administering to at least one tissue or cell selected from the group consisting of: an airway tissue; nose tissue; oral tissue; alveoli tissue; pharynx tissue; trachea tissue; bronchi tissue; carina tissue; bronchi tissue; bronchioles tissue; lung tissue; lobe of a lung tissue; alveoli tissue; nasal passage tissue; nasal epithelium tissue; larynx tissue; bronchi tissue; inhalation tissue; an epithelium cell; an airway epithelium cell; a ciliated cell; a goblet cell; a non-ciliated cell; a basal cell; a lung cell; a nasal cell; a tracheal cell; a bronchial cell; a bronchiolar epithelial cell; an alveolar epithelial cell; and a sinus cell.

48. The method of claim 27, wherein administering is by at least one delivery system selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal aerosol device; a nasal nebulization device; a pressure-driven jet nebulizer; ultrasonic nebulizer; a breath-powered nasal delivery device; a atomized nasal medication device; an inhaler; a powder dispenser; a dry powder generator; an aerosolizer; an intrapulmonary aerosolizer; a sub-miniature aerosolizer; a propellant based metered dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and a combination thereof.

49. The method of claim 27, wherein the method
reduces nasal virus titer at least 10 fold or 100 fold, or
prevents or reduces nasal shedding of virus at least 10 fold or 100 fold.

50. The method of claim 27, wherein the tdsRNA is administered at a dosage of about 25-700 milligram, 20 mg to 200 mg, 50 mg to 150 mg, or 80 mg to 140 mg, per day.

51. The method of claim 27, wherein the subject is a mammal, preferably a host of the virus, and most preferably a human.

52. A delivery system or medical device encompassing a composition of claim 1.

53. The delivery system or medical device of claim 52, wherein the delivery system or medical device is selected from the group consisting of: a nebulizer; a sprayer; a nasal pump; a squeeze bottle; a nasal spray; a syringe sprayer or plunger sprayer (a syringe providing pressure to an attached sprayer or nozzle); a nasal aerosol device; a controlled particle dispersion device; a nasal nebulization device; a pressure-driven jet nebulizer; ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered dose inhaler; a dry powder inhalation device; an instillation device; an intranasal instillation device; an intravesical instillation device; a swab; a pipette; a nasal irrigation device; a nasal rinse; an aerosol device; a metered aerosol device; a pressurized dosage device; a powdered aerosol device; a spray aerosol device; a spray device; a metered spray device; a suspension spray device; and a combination thereof.
